(19) United States
(12) Patent Application Publication
(10) Pub. No.: US 2004/0002474 A1
(43) Pub. Date: Jan. 1, 2004

(54) IFN-alpha homologues

(75) Inventors: Volker Heinrichs, Mountain View, CA (US); Teddy Chen, Belmont, CA (US); Phillip A. Patten, Portola Valley, CA (US)

Correspondence Address: MAXYGEN, INC., INTELLECTUAL PROPERTY DEPARTMENT, 515 GALVESTON DRIVE, RED WOOD CITY, CA, 94063, US

(73) Assignee: Maxygen Inc., Redwood City, CA (US)

(21) Appl. No.: 10/389,674

(22) Filed: Mar. 14, 2003

Related U.S. Application Data
(63) Continuation of application No. 09/685,189, filed on Oct. 6, 2000, now abandoned.
Application No. 09/685,189 is a continuation-in-part of application No. 09/415,183, filed on Oct. 7, 1999, now abandoned.

(51) Int. Cl. A61K 48/00; C07H 21/04; C12P 21/04; A61K 38/21; C07K 14/56; C12N 5/06

(52) U.S. Cl. 514/44; 424/85.7; 435/69.51; 435/320.1; 435/325; 530/351; 536/23.5

(57) ABSTRACT

Alpha interferon homologues (both nucleic acids and polypeptides) are provided. Compositions including these interferon homologue polypeptides and nucleic acids, recombinant cells comprising said homologue polypeptides and nucleic acids, methods of making the new homologues, antibodies to the new homologues, and methods of using the homologues are provided. Integrated systems comprising the sequences of the nucleic acids or polypeptides are also provided.

### CROSS REFERENCE TO RELATED APPLICATIONS

This application is a continuation-in-part application of and claims the benefit of and priority to U.S. patent application Ser. No. 09/145,483, filed Oct. 7, 1999, the disclosure of which is incorporated herein by reference in its entirety for all purposes.

### COPYRIGHT NOTIFICATION

Pursuant to 37 C.F.R. 1.71(e), a portion of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to the generation of new interferon-alpha homologues.

### BACKGROUND OF THE INVENTION

Interferon-alphas are members of the diverse helical-bundle superfamily of cytokine genes (Sprang, S. R. et al. (1993) Curr. Opin. Struct. Biol. 3:815-827). The human interferon-alphas are encoded by a family of over 20 tandemly duplicated nonallelic genes that share 85-98% sequence identity at the amino acid level (Henco, K. et al. (1985) J. Mol. Biol. 185:227-260).

Interferon-alphas have been shown to inhibit various types of cellular proliferation, and are especially useful for the treatment of a variety of cellular proliferation disorders frequently associated with cancer, particularly hematologic malignancies such as leukemias. These proteins have shown antiproliferative activity against multiple myeloma, chronic lymphocytic leukemia, low-grade lymphoma, Kaposi's sarcoma, chronic myelogenous leukemia, renal-cell carcinoma, urinary bladder tumors and ovarian cancers (Bonnem, E. M. et al. (1984) J. Biol. Response Modifiers 3:580; Oldham, R. K. (1985) Hospital Practice 20:71).

Interferon-alphas are also useful against various types of viral infections (Finter, N. B. et al. (1991) Drugs 42(5):749). Interferon-alphas have shown activity against human papillomavirus infection, Hepatitis B, and Hepatitis C infections (Finter, N. B. et al., 1991, supra; Kashima, H. et al. (1988) Laryngoscope 98:334; Dusheiko, G. M. et al. (1986) J. Hematology 3 (Supple. 2):S199; Davis, G L et al. (1989) N. England J. Med. 321:1501). The role of interferons and interferon receptors in the pathogenesis of certain autoimmune and inflammatory diseases has also been investigated (Benoit, P. et al. (1993) J. Immunol. 150(3):707).

Although these proteins possess therapeutic value in the treatment of a number of diseases, they have not been optimized for use as pharmaceuticals. For example, dose-limiting toxicity, receptor cross-reactivity, and short serum half-lives significantly reduce the clinical utility of many of these cytokines (Dusheiko, G. (1997) Hepatology 26:112S-121S; Vial, T. and Descotes, J. (1994) Drug Experience 10:115-150; Funke, I. et al. (1994) Ann. Hematol. 68:49-52; Schomburg, A. et al. (1993) J. Cancer Res. Clin. Oncol. 119:745-755). Diverse and severe side effect profiles which accompany interferon administration include flu-like symptoms, fatigue, neurological disorders including hallucination, fever, hepatic enzyme elevation, and leukopenia (Pontzer, C. H. et al. (1991) Cancer Res. 51:5304; Oldham, 1985, supra).

The existence of abundant naturally occurring sequence diversity within the interferon-alphas (and hence a large sequence space of recombinants) along with the intricacy of interferon-alpha/receptor interactions and variety of therapeutic and prophylactic activities creates an opportunity for the construction of superior interferon homologues.

### SUMMARY OF THE INVENTION

The invention provides novel interferon-alpha (IFN-alpha or IFN-α) homologue polypeptides, nucleic acids encoding the polypeptides and complementary nucleotide sequences thereof, fragments of said polypeptides and nucleic acids, antibodies to the polypeptides, and uses therefor, data sets containing character strings of interferon-alpha homologue sequences, and automated systems for using the character strings.

In one aspect, the invention includes an isolated or recombinant interferon-alpha nucleic acid homologue. Included are a polynucleotide sequences selected from SEQ ID NO:1 to SEQ ID NO:35, or to SEQ ID NO:72 to SEQ ID NO:78, and complementary polynucleotide sequences thereof. Polynucleotide sequences encoding a polypeptide selected from SEQ ID NO:36 to SEQ ID NO:81 or from SEQ ID NO:79 to SEQ ID NO:85, and complementary polynucleotide sequences thereof are also a feature of the invention. Similarly, a polynucleotide sequence which hybridizes under highly stringent conditions over substantially the entire length of any of the preceding polynucleotide sequences is a feature of the present invention. In addition, a polynucleotide sequence comprising a nucleotide fragment of any of the preceding polynucleotide sequences which nucleotide fragment encodes a polypeptide having an antiproliferative activity in a human Daudi cell line- based cell proliferation assay is a feature of the invention. Similarly, a polynucleotide sequence comprising a nucleotide fragment of any of the polynucleotide sequences of the invention described above and below which encodes a polypeptide having antiviral activity in a murine cell line/EMCV-based assay is a feature of the invention.

The invention also includes an isolated or recombinant nucleic acid, comprising a polynucleotide sequence encoding a polypeptide, wherein the polypeptide comprises the amino acid sequence: CDLPQTHSLG-X₁₁-X₁₂-RA-X₁₅-X₁₆-LL-X₁₉-QM-X₂₂-R-X₂₄-S-X₂₆-FSCLKDR-X₃₄-DFG-X₃₈-P-X₄₀-EEFD-X₄₅-X₄₆-X₄₇-FQ-X₅₀-X₅₁-QAI-X₅₅-X₅₆-X₅₇-HE-X₆₀-X₆₁-QQTFN-X₆₇-FSTK-X₇₂-SS-X₇₅-X₇₆-W-X₇₈-X₇₉-X₈₀-LL-X₈₃-K-X₈₅-X₈₆-T-X₈₈-L-X₉₀-QQLN-X₉₅-LEACV-X₁₀₁-Q-X₁₀₃-V-X₁₀₅-X₁₀₆-X₁₀₇-X₁₀₈-TPLMN-X₁₁₄-D-X₁₁₆-ILAV-X₁₂₁-KY-X₁₂₄-QRITLYL-X₁₃₂-E-X₁₃₄-KYSPC-X₁₄₀-WEVVRAEIMRSFSFSTNLQKRLRRKE, or a conservatively substituted variation thereof, where X₁₁ is N or D; X₁₂ is R, S, or K; X₁₅ is L or M; X₁₆ is I, M, or V; X₁₉ is A or G; X₂₂ is G or R; X₂₄ is I or T; X₂₆ is P or H; X₃₄ is H, Y or Q; X₃₈ is F or L; X₄₀ is Q or R; X₄₅ is G or S; X₄₆ is N or H; X₄₇ is Q or R; X₅₀ is K or R; X₅₁ is A or T; X₅₅ is S or F; X₅₆ is V or A; X₅₇ is L or F; X₆₀ is M or I; X₆₁ is I or M; X₆₇ is L or F; X₇₂ is D or N; X₇₅ is A or V; X₇₆ is A or T; X₇₈ is E or D; X₇₉ is Q or E; X₈₀ is S, R, T, or N; X₈₃ is E or D; X₈₅ is F or L; X₈₆ is S or Y; X₈₈ is E or G; X₉₀ is Y, H, N; X₉₅ is D, E, or N; X₁₀₁ is I, M, or V; X₁₀₃ is E or G; X₁₀₅ is G or W; X₁₀₆ is V or M; X₁₀₇ is E, G, or K; X₁₀₈ is E or G; X₁₁₄ is V, E, or G; X₁₁₆ is S or P; X₁₂₁ is K or R; X₁₂₄ is F or L; X₁₃₂ is T, I, or M; X₁₃₄ is K or R; and X₁₄₀ is A or S. Each of the single letters of this amino acid sequence represents a particular amino acid residue according to standard practice known to those of ordinary skill in the art.

A polypeptide having any of the preceding sequences, such as those embodied in SEQ ID NO:36 to SEQ ID NO:54, is also a feature of the invention.

In other embodiments, the encoded polypeptide comprises an amino acid sequence selected from the group consisting of SEQ ID NO:36 to SEQ ID NO:54; and the nucleic acid comprises a polynucleotide sequence selected from the group consisting of SEQ ID NO:1 to SEQ ID NO:19.

The invention also provides polypeptide fragments of any of SEQ NOS:36-70 and SEQ ID NOS:72-79. In one aspect of the invention, such a polypeptide fragment exhibits an antiproliferative activity in a human Daudi cell line-based cell proliferation assay or an antiviral activity in a murine cell line/EMCV-based assay, or both said activities. The human Daudi cell line-based cell proliferation assay and antiviral activity in a murine cell line/EMCV-based assay are described in greater detail below. In yet another aspect, the invention provides a polynucleotide sequence comprising a nucleotide fragment of any nucleic acid of the invention described above and below, wherein said nucleotide fragment encodes a polypeptide fragment that exhibits an antiproliferative activity in a human Daudi cell line-based cell proliferation assay or an antiviral activity in a murine cell line/EMCV-based assay, or both activities, as is described in greater detail below.

The invention also includes an isolated or recombinant nucleic acid comprising a polynucleotide sequence encoding a polypeptide, wherein the polypeptide comprises an amino acid sequence comprising at least 20 contiguous amino acids of any one of SEQ ID NOS:36-70. In other embodiments, the polypeptide of the invention comprises an amino acid sequence comprising one or more of amino acid residues (Tyr or Gln)34, Gly37, Phe38, Lys71, Ala76, Tyr90, Ile32, Arg134, Phe152, Lys160, and Glu166, wherein the numbering of the amino acid residues corresponds to the numbering of residues in the amino acid sequence of SEQ ID NO:36. In various embodiments, the encoded polypeptide of the invention comprises at least 30, at least 50, at least 70, at least 75, at least 100, at least 110, at least 120, at least 130, at least 140, at least 150, at least 155, at least 160, or at least 165 contiguous amino acid residues of any one of SEQ ID NOS:36-70. In other embodiments, the encoded polypeptide is at least 150, at least 155, at least 160, at least 163, or at least 165 amino acids in length. In another embodiment, the encoded polypeptide is about 166 amino acids in length. In yet other embodiments, the encoded polypeptide comprises an amino acid sequence selected from SEQ ID NO:36, SEQ ID NO:37, SEQ ID NO:39, SEQ ID NO:40, SEQ ID NO:41, SEQ ID NO:42, SEQ ID NO:45, and SEQ ID NO:46.

In other embodiments, the invention provides a nucleic acid that comprises a polynucleotide sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:10, and SEQ ID NO:11.

In other embodiments, the polypeptide encoded by any nucleic acid or the invention described herein or a fragment thereof may have antiproliferative activity in a human Daudi cell line-based assay, or antiviral activity in a human WISH cell/EMCV-based assay. In other embodiments, the encoded polypeptide has antiproliferative activity of at least about 8.3×10⁶ units/milligram in the human Daudi cell line-based assay (1 unit is the amount of protein in milligram (mg) required to induce 50% antiproliferative activity), or antiviral activity of at about least 2.1×10⁷ units/milligram (mg) in the human WISH cell/EMCV-based assay (1 unit is the amount of protein in mg required to induce 50% antiviral activity). In other embodiments, the encoded polypeptide can bind to a type I interferon receptor, preferably a human type I interferon receptor, more preferably a human (e.g., type I) interferon-alpha receptor.

The invention also includes a cell comprising any nucleic acid of the invention described herein, or which expresses any polypeptide of the invention noted herein. In one embodiment, the cell expresses a polypeptide encoded by the nucleic acid of the invention as described herein.

The invention also includes a vector comprising any nucleic acid of the invention described above and below. The vector can comprise a plasmid, a cosmid, a phage, or a virus; the vector can be, e.g., an expression vector, a cloning vector, a packaging vector, an integration vector, or the like. The invention also includes a cell transduced by a vector of the invention. The invention also includes compositions comprising any nucleic acid of the invention described above and below, and an excipient, preferably a pharmaceutically acceptable excipient. Cells and transgenic animals which include any polypeptide or nucleic acid of the invention described above and below, e.g., produced by transduction of vector, are a feature of the invention.

The invention also includes compositions produced by digesting one or more of the nucleic acids of the invention described above or below with a restriction endonuclease, an RNAse, or a DNAse; and, compositions produced by incubating one or more nucleic acids described above or below in the presence of deoxyribonucelotide triphosphates and a nucleic acid polymerase, e.g., a thermostable polymerase.

The invention also includes compositions comprising two or more nucleic acids described above or below. The composition may comprise a library of nucleic acids, where the library contains at least about 5, 10, 20 or 50 nucleic acids.

In another aspect, the invention includes an isolated or recombinant polypeptide encoded by any nucleic acid described above or below. In one embodiment, the polypeptide may comprise a sequence selected from SEQ ID NO:36 to SEQ ID NO:70, or SEQ ID NO:79 to SEQ ID NO:85.

The invention also includes a polypeptide comprising at least 50 contiguous amino acids of a protein encoded by a polynucleotide sequence, the polynucleotide sequence selected from the group consisting of: (a) SEQ ID NO:1 to SEQ ID NO:35 or SEQ ID NO:72 to SEQ ID NO:78; (b) a polynucleotide sequence that encodes a polypeptide selected from SEQ ID NO:36 to SEQ ID NO:70 or SEQ ID NO:79 to SEQ ID NO:85; and (c) a complementary sequence of a polynucleotide sequence which hybridizes under highly stringent conditions over substantially the entire length of polynucleotide sequence (a) or (b). In various embodiments, the polypeptide comprises at least about 70, 100, 120, 130, 140, 150, 155, 160, 165, or 166 contiguous amino acids of the encoded protein.

The invention also includes an isolated or recombinant polypeptide comprising an amino acid sequence comprising at least 50 contiguous amino acid residues of any one of SEQ ID NOS:36-70, and one or more of amino acids Ala19, (Tyr or Gln)34, Gly37, Phe38, Lys71, Ala76, Tyr90, Ile132, Arg134, Phe152, Lys160, and Glu166, where the numbering of the amino acids corresponds to that of SEQ ID NO:36. In various embodiments, the polypeptide comprises at least about 50, 70, 75, 100, 110, 120, 130, 140 150, 155, 160, 163, 165, or 166 contiguous amino acids of any one of SEQ ID NOS:36-70. In more preferred embodiments, the polypeptide comprises at least about 50, 70, 75, 100, 110, 120, 130, 140, 150, 155, 160, 163, 165, or 166 contiguous amino acid residues of any one of SEQ ID NO:36, SEQ ID NO:37, SEQ ID NO:39, SEQ ID NO:40, SEQ ID NO:41, SEQ ID NO:42, SEQ ID NO:45, or SEQ ID NO:46. In other embodiments, the polypeptide of the invention is at least about 50, 70, 75, 100, 110, 120, 130, 140, 150, 155, 160, 163, 165, or 166 amino acid residues in length, or is preferably 166 amino acids in length. Longer polypeptides, e.g., which comprise purification tags or the like, are also contemplated. Such polypeptides may display antiproliferative activities in human Daudi cell-line based assay and/or antiviral activities in a human WISH cell/EMCV-based assay.

The invention also includes a polypeptide which specifically binds polyclonal antisera raised against at least one antigen, said at least one antigen comprising a polypeptide sequence selected from an amino acid sequence set forth in SEQ ID NO:36 to SEQ ID NO:70 or SEQ ID NO:79 to SEQ ID NO:85 or a fragment thereof. In particular, the invention provides polypeptides which bind a polyclonal antisera raised against at least one antigen, wherein said at least one antigen comprises at least one amino acid sequence set forth in SEQ ID NO:36 to SEQ ID NO:70 or SEQ ID NO:79 to SEQ ID NO:85, or a fragment of any of these amino sequences, wherein the polyclonal antisera is subtracted with one or more known interferon-alpha polypeptides or proteins, including, e.g., a polypeptide or protein encoded by a nucleic acid having or corresponding to one or more of the following GenBank™ accession numbers: J00210 (alpha-D), J00207 (Alpha-a), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1), and other similar or homologous interferon-alpha nucleic acid sequences presented in GenBank.

Any polypeptide described above or below optionally has antiproliferative activity in a human Daudi cell line-based assay and/or in an antiviral activity in a human WISH cell/EMCV-based assay. Any polypeptide described above or below can have antiproliferative activity of at least about 8.3×10⁶ units/mg in the human Daudi cell line-based assay or antiviral activity of at least about 2.1×10⁷ units/mg in the human WISH cell/EMCV-based assay. In other embodiments, any polypeptide described above or below can bind to a type I interferon receptor, preferably a human type I interferon receptor, more preferably a human interferon-alpha receptor.

In other embodiments, any polypeptide described above or below may further include a secretion/localization sequence, e.g., a signal sequence, an organelle targeting sequence, a membrane localization sequence, and the like. Any polypeptide described herein may further include a sequence that facilitates purification, e.g., an epitope tag (such as, a FLAG epitope), a polyhistidine tag, a GST fusion, and the like. The polypeptide optionally includes a methionine at the N-terminus. Any polypeptide of the invention described herein optionally includes one or more modified amino acids, such as a glycosylated amino acid, a PEG-ylated amino acid, a farnesylated amino acid, an acetylated amino acid, a biotinylated amino acid, a carboxylated amino acid, a phosphorylated amino acid, an acylated amino acid, or the like.

The invention also includes compositions comprising any polypeptide described herein in an excipient, preferably a pharmaceutically acceptable excipient.

The invention also includes an antibody or antisera produced by administering one or more of the polypeptides of the invention described herein to a mammal, wherein the antibody or antisera does not specifically bind to a known alpha-interferon polypeptide or protein, including, e.g., any polypeptide or protein encoded by a nucleic acid having or corresponding to one or more of the following GenBank accession numbers: J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1), and other similar or homologous interferon-alpha sequences presented in GenBank.

The invention also includes antibodies which specifically bind a polypeptide comprising a sequence selected from SEQ ID NO:36 to SEQ ID NO:70 or SEQ ID NO:79 to SEQ ID NO:85. The antibodies are, e.g., polyclonal, monoclonal, chimeric, humanized, single chain, Fab fragments, fragments produced by an Fab expression library, or the like.

Methods for producing the polypeptides of the invention are also included. One such method comprises introducing into a population of cells any nucleic acid described herein, operatively linked to a regulatory sequence effective to produce the encoded polypeptide, culturing the cells in a culture medium to produce the polypeptide, and optionally isolating the polypeptide from the cells or from the culture medium. The nucleic acid may be part of a vector, such as a recombinant expression vector.

The invention also includes a method of inhibiting growth of tumor cells, by contacting the tumor cells with a polypeptide of the invention described herein, thereby inhibiting growth of the tumor cells. In one embodiment, the invention includes a method of inhibiting growth of population of tumor cells comprising contacting the population of tumor cells with an effective amount of a polypeptide of the invention sufficient to inhibit growth of tumor cells in said population of tumor cells, thereby inhibiting growth of tumor cells in said population of cells. In various embodiments, the tumor cells can be human carcinoma cells, human leukemia cells, human T-lymphoma cells, human melanoma cells, other human cancer cells as described herein, and the like. The tumor cells can be in vivo, ex vivo, or in vitro (e.g., cultured cells).

The invention also includes a method of inhibiting the replication of a virus within one or more cells infected by the virus, by contacting one or more of the infected cells with an effective amount of a polypeptide of the invention as described above and below, wherein said amount is sufficient to inhibit viral replication in said one or more infected cells, thereby inhibiting replication of the virus in the one or more cells. In various embodiments, the virus can be an RNA virus, e.g., a human immunodeficiency virus or a hepatitis C virus, or a DNA virus, e.g., a hepatitis B virus. The infected cells can be in vivo, ex vivo, or in vitro (e.g., cultured cells).

The invention also includes a method of treating an autoimmune disorder in a subject in need of such treatment, by administering to the subject an effective amount of a polypeptide of the invention as described herein sufficient to treat the autoimmune disorder. In various embodiments, the autoimmune disorder may be multiple sclerosis, rheumatoid arthritis, lupus erythematosus, type I diabetes, and the like. The invention also includes, in a method of treating a disorder treatable by administration of interferon-alpha to a subject, an improvement comprising administering to the subject an effective amount of a polypeptide of the invention as described herein sufficient to treat said disorder. The disorder treatable by administration of interferon-alpha disorder may be multiple sclerosis, rheumatoid arthritis, lupus erythematosus, type I diabetes, AIDS or AIDS-related complexes, or the like.

In general, nucleic acids and proteins derived by mutation of the sequences herein are a feature of the invention. Similarly, those produced by diversity generation or recursive sequence recombination (RSR) methods (e.g., DNA shuffling) are a feature of the invention. Mutation and recombination methods using the nucleic acids described herein are a feature of the invention. For example, one method of the invention includes recursively recombining one or more nucleic acid sequences of the invention as described above and below with one or more additional nucleic acids (including, but not limited to, those noted herein), each sequence of the one or more additional nucleic acids encoding an interferon-alpha homologue or an amino acid subsequence thereof. The recombining steps are optionally performed in vivo, ex vivo, in silico or in vitro. Said recursive recombination produces at least one library of recombinant interferon-alpha homologue nucleic acids. Also included in the invention are a recombinant interferon-alpha homologue nucleic acid produced by this method, a cell containing the recombinant interferon-alpha homologue nucleic acid, a nucleic acid library produced by this recursive recombination method, a composition comprising two or more of said recombinant interferon-alpha nucleic acids, and a population of cells comprising such recombinant interferon-alpha nucleic acids or containing the library. In one embodiment, the library comprise at least ten such recombinant nucleic acids.

The invention also provides a method of producing a modified or recombinant interferon-alpha homologue nucleic acid that comprises mutating a nucleic acid of the invention as described herein.

Also provided are nucleic acids that encode an interferon-alpha homologue having an increased growth inhibition activity, cytostatic activity, or cytotoxic activity against a population of cells (e.g., cancer cells) relative to the growth inhibition activity cytostatic activity, or cytotoxic activity, respectively, of human interferon-alpha 2a or other known interferon-alpha against the population of cells.

These and other objects and features of the invention will become more fully apparent when the following detailed description is read in conjunction with the accompanying figures.

### BRIEF DESCRIPTION OF THE FIGURES

FIGS. 1A-1E show an alignment of exemplary mature interferon homologue polypeptide sequences (SEQ ID NOS: 36-70 and 79-85) according to the invention.

FIG. 2 shows antiproliferative activities in a human Daudi cell line-based assay and antiviral activities in a human WISH cell/EMCV-based assay of, respectively, exemplary interferon homologues of the present invention relative to the respective antiproliferative and antiviral activities of two control compounds, human interferon alpha-2a (“IFN-α-2a” or “2a”) and consensus human interferon (“IFN-Con1” or “Con1”).

FIGS. 3A, 3B, and 3C illustrate activity profiles of IFN-alpha homologue 3DA11 (SEQ ID NO:40) and control interferons, human interferon alpha-2a (“2a”) and consensus human interferon alpha (“Con1”), against a panel of tumor cell lines. FIG. 3A shows the cell total growth inhibitory activity of IFN-alpha homologue 3DA11 and each control IFN on each respective cell line as reflected in the GI50 value, which is the concentration (μg/ml) of interferon alpha homologue or control IFN alpha at which growth of a particular cell line is inhibited by 50%, as measured by a 50% reduction in the net protein/polypeptide increase in the interferon alpha homologue or control IFN alpha at the end of the incubation period.

FIG. 3B shows the cytostatic activity of IFN-alpha homologue 3DA11 and each control IFN on each cell line of the panel of cell lines. Cytostatic activity refers to an activity capable of suppressing growth and multiplication of cells. Cytostatic activity is assessed as a reflection of the concentration of IFN-alpha homologue 3DA11 or control IFN (μg/ml) at which the growth and/or multiplication of cells of a particular cell line is completely inhibited or suppressed, such that the amount of cellular protein at the end of the incubation period equals the amount of cellular protein at the beginning of the incubation period (“total growth inhibition” or “TGI”).

FIG. 3C illustrates the cytotoxic activity of IFN-alpha homologue 3DA11 and each control IFN on each respective cell line. The cytotoxicity of an agent (e.g., an IFN homologue or IFN compound) is the degree to which the agent possess a specific destructive action on certain cells or the possession of such action. The term typically refers to an agent capable of causing cell death and is used particularly in referring to the lysis of cells by immune phenomena and to agents of compounds that selectively kill dividing cells. In FIG. 3C, cytotoxic activity is illustrated as LC50, the concentration of IFN-alpha homologue 3DA11 (μg/ml) at which a 50% reduction in the net protein increase in control cells (control IFN alpha) at the end of the incubation as compared to that at the beginning of the incubation period is observed, indicating a net loss of cells following addition of the particular interferon. Cytotoxic activity may be assessed as the concentration of IFN-alpha homologue 3DA11 at which, relative to the control cells, 50% of the total number of cells (i.e., total population) of a particular cell line are destroyed or killed.

FIGS. 4A, 4B, 4C, and 4D show the cytostatic activity of selected interferon-alpha homologues of the present invention relative to the cytostatic activities of two control interferon alphas, human interferon-alpha 2a (“2a”) and consensus human interferon-alpha (“Con1”), against a leukemia cell line (RPMI-8226) (FIG. 4A), a lung cancer cell line (NCI-H23) (FIG. 4B), a renal cancer cell line (ACHN) (FIG. 4C), and an ovarian cancer cell line (OVCAR-3) (FIG. 4D), respectively. Cytostatic activity is reflected by a TGI value for a particular interferon alpha (i.e., the concentration of interferon alpha at which cell growth of a cell line is totally inhibited, wherein the amount of cellular protein at the end of the incubation period equals the amount of cellular protein at the beginning of the incubation period).

FIG. 5 presents a comparison of the number of mice (out of a total number of six mice) that survived following administration of doses of 2 μg, 10 μg, and 50 μg of two exemplary IFN-alpha homologues of the present invention (designated “IFN-CH2.2” and “IFN-CH2.3”), doses of 2 μg, 10 μg, and 50 μg of murine IFN-alpha-4, and doses of 2 μg, 10 μg, and 50 μg of human IFN-alpha-2a, respectively. The results shown in FIG. 5 demonstrate that in a murine model system, the improved in vitro antiviral activity of these two exemplary IFN-alpha homologues is maintained and sustained in vivo. Phosphate-buffered saline (PBS) is used as a control.

### DETAILED DESCRIPTION OF THE INVENTION

### Definitions

Unless otherwise defined herein or below in the remainder of the specification, all technical and scientific terms used herein have the same meaning as commonly understood by those of ordinary skill in the art to which the present invention belongs.

A “polynucleotide sequence” is a nucleic acid (which is a polymer of nucleotides (A,C,T,U,G, etc. or naturally occurring or artificial nucleotide analogues)) or a character string representing a nucleic acid, depending on context. Either the given nucleic acid or the complementary nucleic acid can be determined from any specified polynucleotide sequence.

Similarly, an “amino acid sequence” is a polymer of amino acids (a protein, polypeptide, etc.) or a character string representing an amino acid polymer, depending on context. Either the given nucleic acid or the complementary nucleic acid can be determined from any specified polynucleotide sequence.

A nucleic acid, protein, peptide, polypeptide, or other component is “isolated” when it is partially or completely separated from components with which it is normally associated (other peptides, polypeptides, proteins (including complexes, e.g., polymerases and ribosomes which may accompany a native sequence), nucleic acids, cells, synthetic reagents, cellular contaminants, cellular components, etc.), e.g., such as from other components with which it is normally associated in the cell from which it was originally derived. A nucleic acid, polypeptide, or other component is isolated when it is partially or completely recovered or separated from other components of its natural environment such that it is the predominant species present in a composition, mixture, or collection of components (i.e., on a molar basis it is more abundant than any other individual species in the composition). In preferred embodiments, the preparation consists of more than 70%, typically more than 80%, or preferably more than 90% of the isolated species.

In one aspect, a “substantially pure” or “isolated” nucleic acid (e.g., RNA or DNA), polypeptide, protein, or composition also means where the object species (e.g., nucleic acid or polypeptide) comprises at least about 50, 60, or 70 percent by weight (on a molar basis) of all macromolecular species present. A substantially pure or isolated composition can also comprise at least about 80, 90, or 95 percent by weight of all macromolecular species present in the composition. An isolated object species can also be purified to essential homogeneity (contaminant species cannot be detected in the composition by conventional detection methods) wherein the composition consists essentially of derivatives of a single macromolecular species.

The term “isolated nucleic acid” may refer to a nucleic acid (e.g., DNA or RNA) that is not immediately contiguous with both of the coding sequences with which it is immediately contiguous (i.e., one at the 5′ and one at the 3′ end) in the naturally occurring genome of the organism from which the nucleic acid of the invention is derived. Thus, this term includes, e.g., a cDNA or a genomic DNA fragment produced by polymerase chain reaction (PCR) or restriction endonuclease treatment, whether such cDNA or genomic DNA fragment is incorporated into a vector, integrated into the genome of the same or a different species than the organism, including, e.g., a virus, from which it was originally derived, linked to an additional coding sequence to form a hybrid gene encoding a chimeric polypeptide, or independent of any other DNA sequences. The DNA may be double-stranded or single-stranded, sense or antisense.

A nucleic acid or polypeptide is “recombinant” when it is artificial or engineered, or derived from an artificial or engineered protein or nucleic acid. The term “recombinant” when used with reference e.g., to a cell, nucleotide, vector, or polypeptide typically indicates that the cell, nucleotide, or vector has been modified by the introduction of a heterologous (or foreign) nucleic acid or the alteration of a native nucleic acid, or that the polypeptide has been modified by the introduction of a heterologous amino acid, or that the cell is derived from a cell so modified. Recombinant cells express nucleic acid sequences (e.g., genes) that are not found in the native (non-recombinant) form of the cell or express native nucleic acid sequences (e.g., genes) that would be abnormally expressed under-expressed, or not expressed at all. The term “recombinant nucleic acid” (e.g., DNA or RNA) molecule means, for example, a nucleotide sequence that is not naturally occurring or is made by the combatant (for example, artificial combination) of at least two segments of sequence that are not typically included together, not typically associated with one another, or are otherwise typically separated from one another. A recombinant nucleic acid can comprise a nucleic acid molecule formed by the joining together or combination of nucleic acid segments from different sources and/or artificially synthesized. The term “recombinantly produced” refers to an artificial combination usually accomplished by either chemical synthesis means, recursive sequence recombination of nucleic acid segments or other diversity generation methods (such as, e.g., shuffling) of nucleotides, or manipulation of isolated segments of nucleic acids, e.g., by genetic engineering techniques known to those of ordinary skill in the art. “Recombinantly expressed” typically refers to techniques for the production of a recombinant nucleic acid in vitro and transfer of the recombinant nucleic acid into cells in vivo, in vitro, or ex vivo where it may be expressed or propagated. A “recombinant polypeptide” or “recombinant protein” usually refers to polypeptide or protein, respectively, that results from a cloned or recombinant gene or nucleic acid.

A “subsequence” or “fragment” is any portion of an entire sequence, up to and including the complete sequence.

Numbering of a given amino acid or nucleotide polymer “corresponds to numbering” of a selected amino acid polymer or nucleic acid when the position of any given polymer component (amino acid residue, incorporated nucleotide, etc.) is designated by reference to the same residue position in the selected amino acid or nucleotide, rather than by the actual position of the component in the given polymer.

A vector is a composition for facilitating cell transduction by a selected nucleic acid, or expression of the nucleic acid in the cell. Vectors include, e.g., plasmids, cosmids, viruses, YACs, bacteria, poly-lysine, etc. An “expression vector” is a nucleic acid construct, generated recombinantly or synthetically, with a series of specific nucleic acid elements that permit transcription of a particular nucleic acid in a host cell. The expression vector can be part of a plasmid, virus, or nucleic acid fragment. The expression vector typically includes a nucleic acid to be transcribed operably linked to a promoter.

“Substantially an entire length of a polynucleotide or amino acid sequence” refers to at least about 50%, at least about 60%, generally at least about 70%, generally at least about 80%, or typically at least about 90%, 95,%, 96%, 97%, 98%, or 99% or more of a length of an amino acid sequence or nucleic acid sequence.

“A human alpha-interferon receptor” is a receptor which is naturally activated in human cells by an alpha interferon.

“Naturally occurring” as applied to an object refers to the fact that the object can be found in nature. For example, a polypeptide or polynucleotide sequence that is present in an organism, including viruses, that can be isolated from a source in nature and which has not been intentionally modified by man in the laboratory is naturally occurring. In one aspect, a “naturally occurring” nucleic acid (e.g., DNA or RNA) molecule is a nucleic acid molecule that exists in the same state as it exists in nature; that is, the nucleic acid molecule is not isolated, recombinant, or cloned.

As used herein, an “antibody” refers to a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains, respectively. Antibodies exist as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies include single chain antibodies, including single chain Fv (sFv) antibodies in which a variable heavy and a variable light chain are joined together (directly or through a peptide linker) to form a continuous polypeptide.

An “antigen-binding fragment” of an antibody is a peptide or polypeptide fragment of the antibody which binds an antigen. An antigen-binding site is formed by those amino acids of the antibody which contribute to, are involved in, or affect the binding of the antigen. See Scott, T. A. and Mercer, E. I., CONCISE ENCYCLOPEDIA: BIOCHEMISTRY AND MOLECULAR BIOLOGY (de Gruyter, 3d ed. 1997) [hereinafter “Scott, CONCISE ENCYCLOPEDIA”] and Watson, J. D. et al., RECOMBINANT DNA (2d ed. 1992) [hereinafter “Watson, RECOMBINANT DNA”], each of which is incorporated herein by reference in its entirety for all purposes.

An “immunogen” refers to a substance that is capable of provoking an immune response. Examples of immunogens include, e.g., antigens, autoantigens that play a role in induction of autoimmune diseases, and tumor-associated antigens expressed on cancer cells.

An “antigen” is a substance that is capable of eliciting the formation of antibodies in a host or generating a specific population of lymphocytes reactive with that substance. Antigens are typically macromolecules (e.g., proteins and polysaccharides) that are foreign to the host.

The term “immunoassay” includes an assay that uses an antibody or immunogen to bind or specifically bind an antigen. The immunoassay is typically characterized by the use of specific binding properties of a particular antibody to isolate, target, and /or quantify the antigen.

The term “homology” generally refers to the degree of similarity between two or more structures. The term “homologous sequences” refers to regions in macromolecules that have a similar order of monomers. When used in relation to nucleic acid sequences, the term “homology” refers to the degree of similarity between two or more nucleic acid sequences (e.g., genes) or fragments thereof. Typically, the degree of similarity between two or more nucleic acid sequences refers to the degree of similarity of the composition, order, or arrangement of two or more nucleotide bases (or other genotypic feature) of the two or more nucleic acid sequences. The term “homologous nucleic acids” generally refers to nucleic acids comprising nucleotide sequences having a degree of similarity in nucleotide base composition, arrangement, or order. The two or more nucleic acids may be of the same or different species or group. The term “percent homology” when used in relation to nucleic acid sequences, refers generally to a percent degree of similarity between the nucleotide sequences of two or more nucleic acids.

When used in relation to polypeptide (or protein) sequences, the term “homology” refers to the degree of similarity between two or more polypeptide (or protein) sequences (e.g., genes) or fragments thereof. Typically, the degree of similarity between two or more polypeptide (or protein) sequences refers to the degree of similarity of the composition, order, or arrangement of two or more amino acid of the two or more polypeptides (or proteins). The two or more polypeptides (or proteins) may be of the same or different species or group. The term “percent homology” when used in relation to polypeptide (or protein) sequences, refers generally to a percent degree of similarity between the amino acid sequences of two or more polypeptide (or protein) sequences. The term “homologous polypeptides” or “homologous proteins” generally refers to polypeptides or proteins, respectively, that have amino acid sequences and functions that are similar. Such homologous polypeptides or proteins may be related by having amino acid sequences and functions that are similar, but are derived or evolved from different or the same species using the techniques described herein.

The term “subject” as used herein includes, but is not limited to, an organism; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish; and a non-mammalian invertebrate.

The term “pharmaceutical composition” means a composition suitable for pharmaceutical use in a subject, including an animal or human. A pharmaceutical composition generally comprises an effective amount of an active agent and a pharmaceutically acceptable carrier.

The term “effective amount” means a dosage or amount sufficient to produce a desired result. The desired result may comprise an objective or subjective improvement in the recipient of the dosage or amount.

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as a nucleic acid, vector, gene, polypeptide, protein, substance, composition thereof that, when administered to a subject who does not display signs or symptoms of pathology, disease or disorder, or who displays only early signs or symptoms of pathology, disease, or disorder, diminishes, prevents, or decreases the risk of the subject developing a pathology, disease, or disorder. A “prophylactically useful” agent or compound (e.g., nucleic acid or polypeptide) refers to an agent or compound that is useful in diminishing, preventing, treating, or decreasing development of pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such as a nucleic acid, vector, gene, polypeptide, protein, substance, or composition thereof, that eliminates or diminishes signs or symptoms of pathology, disease or disorder, diminishes when administered to a subject suffering from such signs or symptoms. A “therapeutically useful” agent or compound (e.g., nucleic acid or polypeptide) indicates that an agent or compound is useful in diminishing, treating, or eliminating such signs or symptoms of a pathology, disease or disorder.

The term “gene” broadly refers to any segment of DNA associated with a biological function. Genes include coding sequences and/or regulatory sequences required for their expression. Genes also include non-expressed DNA nucleic acid segments that, e.g., form recognition sequences for other proteins.

Generally, the nomenclature used hereafter and the laboratory procedures in cell culture, molecular genetics, molecular biology, nucleic acid chemistry, and protein chemistry described below are those well known and commonly employed by those of ordinary skill in the art. Standard techniques, such as described in Sambrook et al., Molecular Cloning—A Laboratory Manual (2nd Ed.), Vols. 1-3, Cold Spring Harbor Laboratory, Cold Spring Harbor, N.Y., 1989 (hereinafter “Sambrook”) and Current Protocols in Molecular Biology, F. M. Ausubel et al., eds., Current Protocols, a joint venture between Greene Publishing Associates, Inc. and John Wiley & Sons, Inc. (supplemented through 1999) (hereinafter “Ausubel”), are used for recombinant nucleic acid methods, nucleic acid synthesis, cell culture methods, and transgene incorporation, e.g., electroporation, injection, and lipofection. Generally, oligonucleotide synthesis and purification steps are performed according to specifications. The techniques and procedures are generally performed according to conventional methods in the art and various general references which are provided throughout this document. The procedures therein are believed to be well known to those of ordinary skill in the art and are provided for the convenience of the reader.

A variety of additional terms are defined or otherwise characterized herein.

### Polynucleotides of the Invention

Interferon-alpha Homologue Sequences

The invention provides isolated or recombinant interferon-alpha homologue polypeptides, and isolated or recombinant polynucleotides encoding the polypeptides.

As described in more detail below, in accordance with the present invention, polynucleotide sequences which encode novel interferon-alpha homologue polypeptides, nucleotide sequences (e.g., subsequences) that encode fragments of interferon-alpha homologue polypeptides, and nucleotide sequences that encode related fusion polypeptides or proteins, or functional equivalents thereof, are collectively referred to herein as “interferon-alpha homologues,” “interferon homologue nucleic acids,” “IFN-alpha homologues,” “IFN homologues,” “IFN nucleic acids,” “interferon homologues,” “interferon nucleic acids,” “recombinant interferon-alpha,” “recombinant interferon-alpha nucleic acids,” “nucleic acids of the invention,” “polynucleotides of the invention,” or “nucleotides of the invention.” Polynucleotide, nucleotide are nucleic acid fragments of each of the preceding terms are also intended to be included and encompassed in polynucleotides, nucleotides, and nucleic acids of the invention. The term “nucleic acid” is used interchangeable with the term “nucleotide.”

Polynucleotides encoding the polypeptides of the invention were discovered in libraries of shuffled interferon-alpha related sequences. The library members were screened for antiproliferative activity against human tumor cell lines and, in some cases, assayed for antiviral activity against virus-infected human cells. A subset of the sequences provided herein were discovered in shuffled libraries screened for antiviral activity against virus-infected mouse cells. Coding sequences for interferon homologues were identified as described in the examples.

Briefly, libraries of shuffled mature interferon-alpha coding sequences were introduced into E. coli. Colonies were screened in a high-throughput antiproliferative activity assay against a human Daudi tumor cell line as described in Example 1, and colonies expressing active polypeptides were selected, re-screened, and expression levels determined. DNA from selected colonies was isolated and re-shuffled to create secondary libraries. The secondary libraries were introduced into E. coli and screened for antiproliferative activity in the human Daudi cell line-based cell proliferation assay. DNA from colonies selected from the primary and secondary library screens were transduced into Chinese hamster ovary (CHO) cells, and stable cell lines were generated. CHO-expressed proteins were purified, quantitated, and assayed for antiproliferative activity using the human Daudi cell line, and optionally, for antiviral activity using encephalomyocarditis virus (EMCV)-infected human WISH cells, as described in Example 1. Exemplary shuffled nucleic acids which encode interferon-alpha homologue polypeptides having antiproliferative activity in the human Daudi cell line-based assay are identified herein as SEQ ID NO:1 to SEQ ID NO:35, which encode mature interferon-alpha homologue polypeptides identified herein as SEQ ID NO:36 to SEQ ID NO:70, respectively. Libraries of shuffled mature interferon-alpha coding sequences were also screened in a high-throughput antiviral activity screen against EMCV-infected mouse cells. Exemplary shuffled nucleic acids which encode polypeptides having antiviral activity in the murine cell/EMCV-based assay are identified herein as SEQ ID NO:72 to SEQ ID NO:78, which encode mature interferon homologue polypeptides identified herein as SEQ ID NO:79 to SEQ ID NO:85.

In another aspect, the invention provides an isolated or recombinant nucleic acid that comprises a polynucleotide sequence selected from the group of: (a) SEQ ID NO:1 to SEQ ID NO:35, or a complementary polynucleotide sequence thereof; (b) a polynucleotide sequence encoding a polypeptide selected from SEQ ID NO:36 to SEQ ID NO:71, or a complementary polynucleotide sequence thereof; (c) a polynucleotide sequence which hybridizes under at least stringent or at least highly stringent hybridization conditions (or ultra-high stringent or ultra-ultra- high stringent hybridization conditions) over substantially the entire length of polynucleotide sequence (a) or (b), or with a 50, 120, 130, 140, 145, 150, 155, 160, or 165 nucleotide base subsequence or fragment of a polynucleotide sequence of (a) or (b); and (d) a polynucleotide sequence comprising a fragment of (a), (b), or (c), which fragment encodes all or a part of a polypeptide having an antiproliferative activity in a human Daudi cell line-based assay or an antiviral activity in an assay known in the art for measuring antiviral activity.

In another aspect, the invention provides an isolated or recombinant nucleic acid that comprises a polynucleotide sequence selected from the group of: (a) SEQ ID NO:72 to SEQ ID NO:78, or a complementary polynucleotide sequence thereof; (b) a polynucleotide sequence encoding a polypeptide selected from SEQ ID NO:79 to SEQ ID NO:85, or a complementary polynucleotide sequence thereof; (c) a polynucleotide sequence which hybridizes under at least stringent or at least highly stringent hybridization conditions (or ultra-high stringent or ultra-ultra- high stringent hybridization conditions) over substantially the entire length of polynucleotide sequence (a) or (b), or with a 50, 120, 130, 140, 145, 150, 155, 160, or 165 nucleotide base subsequence or fragment of a polynucleotide sequence of (a) or (b); and (d) a polynucleotide sequence comprising a fragment of (a), (b), or (c), which fragment encodes all or a part of a polypeptide having an antiproliferative activity in a human Daudi cell line-based assay or an antiviral activity in a murine cell line/EMCV-based assay.

The present invention also includes a mature interferon-alpha homologue polypeptide comprising the amino acid identified herein as SEQ ID NO:71 and a polynucleotide sequence encoding said polypeptide or a fragment of said polypeptide having an antiproliferative activity in the human Daudi cell line-based assay and/or an antiviral activity in the murine cell/EMCV-based assay.

The invention also includes an isolated or recombinant nucleic acid comprising a polynucleotide sequence encoding a polypeptide, wherein the polypeptide comprises the amino acid sequence: CDLPQTHSLG-X₁₁-X₁₂-RA-X₁₅-X₁₆-LL-X₁₉-QM-X₂₂-R-X₂₄-S-X₂₆-FSCLKDR-X₃₄-DFG-X₃₈-P-X₄₀-EEFD-X₄₅-X₄₆-X₄₇-FQ-X₅₀-X₅₁ -QAI-X₅₅-X₅₆-X₅₇-HE-X₆₀-X₆₁-QQTFN-X₆₇-FSTK-X₇₂-SS-X₇₅-X₇₆-W-X₇₈-X₇₉-X₈₀-LL-X₈₃-K-X₈₅-X₈₆-T-X₈₈-L-X₉₀-QQLN-X₉₅-LEACV-X₁₀₁-Q-X₁₀₃-V-X₁₀₅-X₁₀₇-X₁₀₇-X₁₀₈-TPLMN-X₁₁₄-D-X₁₁₆-ILAV-X₁₂₁-KY-X₁₂₄-QRITLYL-X₁₃₂-E-X₁₃₄-KYSPC-X₁₄₀-WEVVRAEIMRSFSFSTNLQKRLRRKE, or a conservatively substituted variation thereof, where X₁₁ is N or D; X₁₂ is R, S, or K; X₁₅ is L or M; X₁₆ is I, M, or V; X₁₉ is A or G; X₂₂ is G or R; X₂₄ is I or T; X₂₆ is P or H; X₃₄ is H, Y or Q; X₃₈ is F or L; X₄₀ is Q or R; X₄₅ is G or S; X₄₆ is N or H; X₄₇ is Q or R; X₅₀ is K or R; X₅₁ is A or T; X₅₅ is S or F; X₅₆ is V or A; X₅₇ is L or F; X₆₀ is M or I; X₆₁ is I or M; X₆₇ is L or F; X₇₂ is D or N; X₇₅ is A or V; X₇₆ is A or T; X₇₈ is E or D; X₇₉ is Q or E; X₈₀ is S, R, T, or N; X₈₃ is E or D; X₈₅ is F or L; X₈₆ is S or Y; X₈₈ is E or G; X₉₀ is Y, H, N; X₉₅ is D, E, or N; X₁₀₁ is I, M, or V; X₁₀₃ is E or G; X₁₀₅ is G or W; X₁₀₆ is V or M; X₁₀₇ is E, G, or K; X₁₀₈ is E or G; X₁₁₄ is V, E, or G; X₁₁₆ is S or P; X₁₂₁ is K or R; X₁₂₄ is F or L; X₁₃₂ is T, I, or M; X₁₃₄ is K or R; and X₁₄₀ is A or S. Each of the single letters of this amino acid sequence represents a particular amino acid residue according to standard practice known to those of ordinary skill in the art. Such polypeptides having an antiproliferative activity in the human Daudi cell line-based assay (e.g., at least about 8.3×10⁶ units/mg) and/or an antiviral activities in a human WISH cell/EMCV-based assay (at least about 2.1×10⁷ units/mg).

As described in greater detail below, the polynucleotides of the invention are useful in for a variety of applications, including, but not limited to, as therapeutic and prophylactic agents in methods of in vivo and ex vivo treatment of a variety of diseases, disorders, and conditions in a variety of subjects; for use in in vitro methods, such as diagnostic methods, to detect, diagnose, and treat a variety of diseases, disorders, and conditions in a variety of subjects; for use in, e.g., gene therapy; as therapeutics and prophylactics, e.g., for use in methods of therapeutic and prophylactic treatment of a disease, disorder or condition; as immunogens; for use in diagnostic and screening assays; and as diagnostic probes for the presence of complementary or partially complementary nucleic acids (including for detection of IFN-alpha coding nucleic acids).

Making Polynucleotides of the Invention

Polynucleotides and oligonucleotides of the invention can be prepared by standard solid-phase methods, according to known synthetic methods. Typically, fragments of up to about 20, 30, 40, 50, 60, 70, 80, 90, and/or 100 nucleotide bases are individually synthesized, then joined (e.g., by enzymatic or chemical ligation methods, or polymerase mediated recombination methods) to form essentially any desired continuous sequence. In another aspect, nucleotide fragments of greater than 100 nucleotide bases (e.g., 150, 180, 200, 210, 240, 270, 300, 330, 360, 390, 400, 420, 450, 465, 474, 470, 475, 489, 490, 495, 496 bases) are individually synthesized, then joined (e.g., by enzymatic or chemical ligation methods, or polymerase mediated recombination methods) to form essentially any desired continuous sequence. example, the polynucleotides and oligonucleotides of the invention, including fragments thereof (and those as described herein), can be prepared by chemical synthesis using, e.g., the classical phosphoramidite method described by Beaucage et al. (1981) Tetrahedron Letters 22:1859-69, or the method described by Matthes et al. (1984) EMBO J. 3:801-05., e.g., as is typically practiced in automated synthetic methods. According to the phosphoramidite method, oligonucleotides are synthesized, e.g., in an automatic DNA synthesizer, purified, annealed, ligated and cloned in appropriate vectors.

In addition, essentially any nucleic acid can be custom ordered from any of a variety of commercial sources, such as The Midland Certified Reagent Company (mcrc@oligos.com), The Great American Gene Company (http://www.genco.com), ExpressGen Inc. (www.expressgen.com), Operon Technologies Inc. (Alameda, Calif.) and many others. Similarly, peptides and antibodies can be custom ordered from any of a variety of sources, such as PeptidoGenic (pkim@ccnet.com), HTI Bio-products, inc. (http://www.htibio.com), BMA Biomedicals Ltd. (U.K.), Bio.Synthesis, Inc., and many others.

Certain polynucleotides of the invention may also obtained by screening cDNA libraries (e.g., libraries generated by recombining homologous nucleic acids as in typical diversity generation methods, such as, e.g., shuffling methods) using oligonucleotide probes which can hybridize to or PCR-amplify polynucleotides which encode the interferon homologue polypeptides and fragments of those polypeptides. Procedures for screening and isolating cDNA clones are well-known to those of skill in the art. Such techniques are described in, for example, Sambrook et al., Molecular Cloning—A Laboratory Manual (2nd Ed.), Vols. 1-3, Cold Spring Harbor Laboratory, Cold Spring Harbor, N.Y., 1989 (hereinafter “Sambrook”) and Current Protocols in Molecular Biology, F. M. Ausubel et al., eds., Current Protocols, a joint venture between Greene Publishing Associates, Inc. and John Wiley & Sons, Inc. (supplemented through 1999) (hereinafter “Ausubel”).

As described in more detail herein, the polynucleotides of the invention include sequences which encode novel mature interferon-alpha homologues and sequences complementary to the coding sequences, and novel fragments of such coding sequences and complements thereof. The polynucleotides can be in the form of RNA or in the form of DNA, and include mRNA, cRNA, synthetic RNA and DNA, and cDNA. The polynucleotides can be double-stranded or single-stranded, and if single-stranded, can be the coding strand or the non-coding (anti-sense, complementary) strand. The polynucleotides optionally include the coding sequence of an interferon-alpha homologue (i) in isolation, (ii) in combination with additional coding sequence, so as to encode, e.g., a fusion protein, a pre-protein, a prepro-protein, or the like, (iii) in combination with non-coding sequences, such as introns, control elements such as a promoter, a terminator element, or 5′ and/or 3′ untranslated regions effective for expression of the coding sequence in a suitable host, and/or (iv) in a vector or host environment in which the interferon-alpha homologue coding sequence is a heterologous nucleic acid sequence or gene. Sequences can also be found in combination with typical compositional formulations of nucleic acids, including in the presence of carriers, buffers, adjuvants, excipients and the like.

The term DNA or RNA encoding the respective interferon-alpha homologue polypeptide includes any oligodeoxynucleotide or oligodeoxyribonucleotide sequence which, upon expression in an appropriate host cell, results in production of an interferon-alpha homologue polypeptide of the invention. The DNA or RNA can be produced in an appropriate host cell, or in a cell-free (in vitro) system, or can be produced synthetically (e.g., by an amplification technique such as PCR) or chemically.

Using Polynucleotides of the Invention

The polynucleotides of the invention have a variety of uses in, for example: recombinant production (i.e., expression) of the interferon-alpha homologue polypeptides of the invention; as therapeutics and prophylactics, e.g., for use in methods of therapeutic and prophylactic treatment of a disease, disorder or condition; for use in, gene therapy methods and related applications;; as immunogens; for use in diagnostic and screening assays; as diagnostic probes for the presence of complementary or partially complementary nucleic acids (including for detection of natural IFN-alpha coding nucleic acids); as substrates for further reactions, e.g., shuffling reactions or mutation reactions to produce new and/or improved IFN-alpha homologues, and the like.

### Expression of Polypeptides

In accordance with the present invention, polynucleotide sequences which encode novel and/or mature interferon-alpha homologues, fragments of interferon-alpha proteins, related fusion proteins, or functional equivalents thereof, are collectively referred to herein as “interferon-alpha homologue polypeptides,” “interferon-alpha homologue proteins,” or “interferon-alpha homologues,” “interferon homologues,” “IFN-alpha homologues,” “IFN homologues”, “IFN polypeptides,” “IFN proteins” “polypeptides of the invention,” or “proteins of the invention.” Polypeptide or amino acid fragments of each of the preceding terms are also intended to be included and encompassed in the polypeptides or proteins of the invention. Such polynucleotide sequences of the invention are used in recombinant DNA (or RNA) molecules that direct the expression of the interferon-alpha homologue polypeptides in appropriate host cells. Due to the inherent degeneracy of the genetic code, other nucleic acid sequences which encode substantially the same or a functionally equivalent amino acid sequence are also used to clone and express the interferon homologues.

Modified Coding Sequences

As will be understood by those of skill in the art, it can be advantageous to modify a coding sequence (including, e.g., a nucleotide sequence encoding an interferon-alpha homologue of the invention or a fragment thereof) to enhance its expression in a particular host. The genetic code is redundant with 64 possible codons, but most organisms preferentially use a subset of these codons. The codons that are utilized most often in a species are called optimal codons, and those not utilized very often are classified as rare or low-usage codons (see, e.g., Zhang S. P. et al. (1991) Gene 105:61-72). Codons can be substituted to reflect the preferred codon usage of the host, a process called “codon optimization” or “controlling for species codon bias.”

Optimized coding sequence containing codons preferred by a particular prokaryotic or eukaryotic host (see also Murray, E. et al. (1989) Nuc. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, preferred stop codons for S. cerevisiae and mammals are UAA and UGA, respectively. The preferred stop codon for monocotyledonous plants is UGA, whereas insects and E. coli prefer to use UAA as the stop codon (Dalphin M. E. et al. (1996) Nuc. Acids Res. 24:216-218).

The polynucleotide sequences of the present invention can be engineered in order to alter an interferon homologue coding sequence for a variety of reasons, including but not limited to, alterations which modify the cloning, processing and/or expression of the gene product. For example, alterations may be introduced using techniques which are well known in the art, e.g., site-directed mutagenesis, to insert new restriction sites, to alter glycosylation patterns, to change codon preference, to introduce splice sites, etc.

Vectors, Promoters and Expression Systems

The present invention also includes recombinant constructs comprising one or more of the nucleic acid sequences as broadly described herein (e.g., those encoding an interferon-alpha homologue of the invention or a fragment thereof). The constructs comprise a vector, such as, a plasmid, a cosmid, a phage, a virus (including a retrovirus), a bacterial artificial chromosome (BAC), a yeast artificial chromosome (YAC), and the like, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available.

General texts which describe molecular biological techniques useful herein, including the use of vectors, promoters and many other relevant topics, include Juo, P-S., CONCISE DICTIONARY OF BIOMEDICAL AND MOLECULAR BIOLOGY (CRC Press 1996); Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); Hale & Marham, THE HARPER COLLINS DICTIONARY OF BIOLOGY (1991); Scott and Mercer, CONCISE ENCYCLOPEDIA OF BIOCHEMISTRY AND MOLECULAR BIOLOGY (3d ed. 1997); Berger and Kimmel, Guide to Molecular Cloning Techniques, Methods in Enzymology, volume 152 Academic Press, Inc., San Diego, Calif. (hereinafter “Berger”); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc. (supplemented through 1999) (“Ausubel”)). Examples of techniques sufficient to direct persons of skill through in vitro amplification methods, including the polymerase chain reaction (PCR), the ligase chain reaction (LCR), Qβ-replicase amplification and other RNA polymerase mediated techniques (e.g., NASBA), e.g., for the production of the homologous nucleic acids of the invention are found in Berger, Sambrook, and Ausubel, as well as Mullis et al. (1987) U.S. Pat. No. 4,683,202; U.S. Pat. No. 4,683,195, issued Jul. 28, 1997; PCR Protocols: A Guide to Methods and Applications (Innis et al., eds.) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Nat'l Acad. Sci. USA 86, 1173; Guatelli et al. (1990) Proc. Nat'l Acad. Sci. USA 87, 1874; Lomell et al. (1989) J. Clin. Chem. 35, 1826; Landegren et al. (1988) Science 241, 1077-1080; Van Brunt (1990) Biotechnology 8, 291-294; Wu and Wallace (1989) Gene 4, 560; Barringer et al. (1990) Gene 89, 117, and Sooknanan and Malek (1995) Biotechnology 13:563-564.

PCR generally refers to a procedure wherein minute amounts of a specific piece of nucleic acid, RNA, and/or DNA, are amplified by methods well known in the art (see, e.g., U.S. Pat. No. 4,683,195 and other references above). Generally, sequence information from the ends of the region of interest or beyond is used, for design of oligonucleotide primers. Such primers will be identical or similar in sequence to the opposite strands of the template to be amplified. The 5′ terminal nucleotides of the opposite strands may coincide with the ends of the amplified material. PCR may be used to amplify specific RNA or specific DNA sequences, recombinant DNA or RNA sequences, DNA and RNA sequences from total genomic DNA, and cDNA transcribed from total cellular RNA, bacteriophage or plasmid sequences, etc. PCR is one example, but not the only example, of a nucleic acid polymerase reaction method for amplifying a nucleic acid test sample comprising the use of a another (e.g., known) nucleic acid as a primer. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369:684-685 and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See Ausubel, Sambrook and Berger, all supra.

The present invention also relates to host cells which are transduced with vectors of the invention, and the production of polypeptides of the invention (including fragments thereof) by recombinant techniques. Host cells are genetically engineered (i.e., transduced, transformed or transfected) with the vectors of this invention, which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants, or amplifying the interferon homologue gene. The culture conditions, such as temperature, pH and the like, are those previously used with the host cell selected for expression, and will be apparent to those skilled in the art and in the references cited herein, including, e.g., Freshney (1994) Culture of Animal Cells, a Manual of Basic Technique, 3d ed., Wiley-Liss, New York and the references cited therein.

The interferon homologue polypeptides and proteins of the invention can also be produced in non-animal cells such as plants, yeast, fungi, bacteria and the like. In addition to Sambrook, Berger and Ausubel, details regarding cell culture can be found in Payne et al. (1992) Plant Cell and Tissue Culture in Liquid Systems, John Wiley & Sons, Inc. New York, N.Y.; Gamborg and Phillips (eds.) (1995) Plant Cell, Tissue and Organ Culture; Fundamental Methods, Springer Lab Manual, Springer-Verlag (Berlin Heidelberg New York) and Atlas and Parks (eds.) The Handbook of Microbiological Media (1993) CRC Press, Boca Raton, Fla.

The polynucleotides of the present invention may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others. Any vector that transducers genetic material into a cell, and, if replication is desired, which is replicable and viable in the relevant host can be used.

The nucleic acid sequence in the expression vector is operatively linked to an appropriate transcription control sequence (promoter) to direct mRNA synthesis. Examples of such promoters include: LTR or SV40 promoter, E. coli lac or trp promoter, phage lambda P_{L} promoter, and other promoters known to control expression of genes in prokaryotic or eukaryotic cells or their viruses. The expression vector also contains a ribosome binding site for translation initiation, and a transcription terminator. The vector optionally includes appropriate sequences for amplifying expression. In addition, the expression vectors optionally comprise one or more selectable marker genes to provide a phenotypic trait for selection of transformed host cells, such as dihydrofolate reductase or neomycin resistance for eukaryotic cell culture, or such as tetracycline or ampicillin resistance in E. coli.

The vector containing the appropriate DNA sequence as described herein, as well as an appropriate promoter or control sequence, may be employed to transform an appropriate host to permit the host to express the protein. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as CHO, COS, BHK, HEK 293 or Bowes melanoma; plant cells, etc. It is understood that not all cells or cell lines need to be capable of producing fully functional interferon homologues; for example, antigenic fragments of an interferon homologue may be produced in a bacterial or other expression system. The invention is not limited by the host cells employed.

In bacterial systems, a number of expression vectors may be selected depending upon the use intended for the interferon homologue. For example, when large quantities of interferon homologue or fragments thereof are needed for the induction of antibodies, vectors which direct high level expression of fusion proteins that are readily purified may be desirable. Such vectors include, but are not limited to, multifunctional E. coli cloning and expression vectors such as BLUESCRW™ (Stratagene), in which the interferon homologue coding sequence may be ligated into the vector in-frame with sequences for the amino-terminal Met and the subsequent 7 residues of beta-galactosidase so that a hybrid protein is produced; pIN vectors (Van Heeke & Schuster (1989) J. Biol. Chem. 264:5503-5509); pET vectors (Novagen, Madison, Wis.); and the like.

Similarly, in the yeast Saccharomyces cerevisiae a number of vectors containing constitutive or inducible promoters such as alpha factor, alcohol oxidase and PGH may be used for production of the interferon homologue proteins of the invention. For reviews, see Ausubel et al. (supra) and Grant et al. (1987; Methods in Enzymology 153:516-544).

In mammalian host cells, a number expression systems, such as viral-based systems, may be utilized. In cases where an adenovirus is used as an expression vector, a coding sequence is optionally ligated into an adenovirus transcription/translation complex consisting of the late promoter and tripartite leader sequence. Insertion in a nonessential E1 or E3 region of the viral genome will result in a viable virus capable of expressing interferon homologue in infected host cells (Logan and Shenk (1984) Proc. Natl. Acad. Sci. 81:3655-3659). In addition, transcription enhancers, such as the rous sarcoma virus (RSV) enhancer, may be used to increase expression in mammalian host cells.

Additional Expression Elements

Specific initiation signals can aid in efficient translation of an interferon homologue coding sequence. These signals can include, e.g., the ATG initiation codon and adjacent sequences. In cases where interferon homologue coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D. et al. (1994) Results Probl. Cell Differ. 20:125-62; Bittner et al. (1987) Methods in Enzymol. 153:516-544).

Secretion/Localization Sequences

Polynucleotides of the invention can also be fused, for example, in-frame to nucleic acid encoding a secretion/localization sequence, to target polypeptide expression to a desired cellular compartment, membrane, or organelle, or to direct polypeptide secretion to the periplasmic space or into the cell culture media. Such sequences are known to those of skill, and include secretion leader peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like. Polypeptides expressed by such polynucleotides of the invention may include the amino acid sequence corresponding to the secretion and/or localization sequence(s).

Expression Hosts

In a further embodiment, the present invention relates to host cells containing the above-described constructs. The host cell can be a eukaryotic cell, such as a mammalian cell, a yeast cell, or a plant cell, or the host cell can be a prokaryotic cell, such as a bacterial cell. Introduction of the construct into the host cell can be effected by calcium phosphate transfection, DEAE-Dextran mediated transfection, electroporation, or other common techniques (Davis, L., Dibner, M., and Battey, I. (1986) Basic Methods in Molecular Biology). The cell may include a nucleic acid of the invention, said nucleic acid encoding a polypeptide, wherein said cells expresses a polypeptide (e.g., an interferon-alpha homologue polypeptide having an antiviral or anti-proliferative activity as measured by the assays described herein). The invention also includes a vector comprising any nucleic acid of the invention described herein and includes a cell transduced by such a vector. Furthermore, Cells and transgenic animals which include any polypeptide or nucleic acid above or throughout this specification, e.g., produced by transduction of a vector of the invention, are an additional feature of the invention.

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “pre” or a “prepro” form of the protein may also be important for correct insertion, folding and/or function. Different host cells such as CHO, HeLa, BHK, MDCK, 293, W138, etc. have specific cellular machinery and characteristic mechanisms for such post-translational activities and may be chosen to ensure the correct modification and processing of the introduced, foreign protein.

For long-term, high-yield production of recombinant proteins, stable expression can be used. For example, cell lines which stably express a polypeptide of the invention are transduced using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. For example, resistant clumps of stably transformed cells can be proliferated using tissue culture techniques appropriate to the cell type.

Host cells transformed with a nucleotide sequence encoding a polypeptide of the invention are optionally cultured under conditions suitable for the expression and recovery of the encoded protein from cell culture. The protein or fragment thereof produced by a recombinant cell may be secreted, membrane-bound, or contained intracellularly, depending on the sequence and/or the vector used. As will be understood by those of skill in the art, expression vectors containing polynucleotides encoding mature interferon homologues of the invention can be designed with signal sequences which direct secretion of the mature polypeptides through a prokaryotic or eukaryotic cell membrane.

Additional Polypeptide Sequences

The polynucleotides of the present invention may also comprise a coding sequence fused in-frame to a marker sequence which, e.g., facilitates purification of the encoded polypeptide of the invention. Such purification facilitating domains include, but are not limited to, metal chelating peptides such as histidine-tryptophan modules that allow purification on immobilized metals, a sequence which binds glutathione (e.g., GST), a hemagglutinin (HA) tag (corresponding to an epitope derived from the influenza hemagglutinin protein; Wilson, I. et al. (1984) Cell 37:767), maltose binding protein sequences, the FLAG epitope utilized in the FLAGS extension/affinity purification system (Immunex Corp., Seattle, Wash.), and the like. The inclusion of a protease-cleavable polypeptide linker sequence between the purification domain and the interferon homologue sequence is useful to facilitate purification. One expression vector contemplated for use in the compositions and methods described herein provides for expression of a fusion protein comprising a polypeptide of the invention fused to a polyhistidine region separated by an enterokinase cleavage site. The histidine residues facilitate purification on IMIAC (immobilized metal ion affinity chromatography, as described in Porath et al. (1992) Protein Expression and Purification 3:263-281), while the enterokinase cleavage site provides a means for separating the interferon homologue polypeptide from the fusion protein. pGEX vectors (Promega; Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Polypeptide Production and Recovery

Following transduction of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well know to those skilled in the art.

As noted, many references are available for the culture and production of many cells, including cells of bacterial, plant, animal (especially mammalian).and archebacterial origin. See, e.g., Sambrook, Ausubel, and Berger (all supra), as well as Freshney (1994) Culture of Animal Cells, a Manual of Basic Technique, third edition, Wiley- Liss, New York and the references cited therein; Doyle and Griffiths (1997) Mammalian Cell Culture: Essential Techniques, John Wiley and Sons, NY; Humason (1979) Animal Tissue Techniques, 4th edition, W. H. Freeman and Company; and Ricciardelli et al. (1989) In vitro Cell Dev. Biol. 25:1016-1024. For plant cell culture and regeneration, Payne et al. (1992) Plant Cell and Tissue Culture in Liquid Systems, John Wiley & Sons, Inc., New York, N.Y.; Gamborg and Phillips (eds.) (1995) Plant Cell, Tissue and Organ Culture; Fundamental Methods Springer Lab Manual, Springer-Verlag (Berlin Heidelberg New York) and Plant Molecular Biology (1993) R. R. D. Croy, ed., Bios Scientific Publishers, Oxford, U.K. ISBN 0 12 198370 6. Cell culture media in general are set forth in Atlas and Parks (eds.) The Handbook of Microbiological Media (1993) CRC Press, Boca Raton, Fla. Additional information for cell culture is found in available commercial literature such as the Life Science Research Cell Culture Catalogue (1998) from Sigma-Aldrich, Inc. (St. Louis, Mo.) (“Sigma-LSRCCC”) and, e.g., the Plant Culture Catalogue and supplement (1997) also from Sigma-Aldrich, Inc. (St. Louis, Mo.) (“Sigma-PCCS”).

Polypeptides of the invention can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature protein. Finally, high performance liquid chromatography (HPLC) can be employed in the final purification steps. In addition to the references noted supra, a variety of purification methods are well known in the art, including, e.g., those set forth in Sandana (1997) Bioseparation of Proteins, Academic Press, Inc.; and Bollag et al. (1996) Protein Methods, 2^{nd} Edition, Wiley-Liss, NY; Walker (1996) The Protein Protocols Handbook, Humana Press, NJ, Harris and Angal (1990) Protein Purification Applications: A Practical Approach, IRL Press at Oxford, Oxford, England; Harris and Angal, Protein Purification Methods: A Practical Approach, IRL Press at Oxford, Oxford, England; Scopes (1993) Protein Purification: Principles and Practice 3^{rd} Edition, Springer Verlag, NY; Janson and Ryden (1998) Protein Purification: Principles, High Resolution Methods and Applications, Second Edition, Wiley-VCH, NY; and Walker (1998) Protein Protocols on CD-ROM, Humana Press, NJ.

In vitro Expression Systems

Cell-free transcription/translation systems can also be employed to produce polypeptides using DNAs or RNAs of the present invention. Several such systems are commercially available. A general guide to in vitro transcription and translation protocols is found in Tymms (1995) In vitro Transcription and Translation Protocols: Methods in Molecular Biology, Volume 37, Garland Publishing, NY.

Modified Amino Acids

Polypeptides of the invention may contain one or more modified amino acids. The presence of modified amino acids may be advantageous in, for example, (a) increasing polypeptide serum half-life, (b) reducing polypeptide antigenicity, (c) increasing polypeptide storage stability. Amino acid(s) are modified, for example, co-translationally or post-translationally during recombinant production (e.g., N-linked glycosylation at N-X-S/T motifs during expression in mammalian cells) or modified by synthetic means.

Non-limiting examples of a modified amino acid include a glycosylated amino acid, a sulfated amino acid, a prenylated (e.g., farnesylated, geranylgeranylated) amino acid, an acetylated amino acid, an acylated amino acid, a PEG-ylated amino acid, a biotinylated amino acid, a carboxylated amino acid, a phosphorylated amino acid, and the like. References adequate to guide one of skill in the modification of amino acids are replete throughout the literature. Example protocols are found in Walker (1998) Protein Protocols on CD-ROM Human Press, Towata, N.J.

The polynucleotides and polypeptides of the invention have a variety of uses, including, but not limited to, for example: in recombinant production (i.e., expression) of the recombinant interferon-alpha homologues of the invention; as therapeutic and prophylactic agents in methods of in vivo and ex vivo treatment of a variety of diseases, disorders, and conditions in a variety of subjects; for use in in vitro methods, such as diagnostic and screening methods, to detect, diagnose, and treat a variety of diseases, disorders, and conditions (e.g., cancers, viral-based disorders, angiogenic-based disorders) in a variety of subjects (e.g., mammals); as immunogens; in gene therapy methods and DNA- or RNA-based delivery methods to deliver or administer ill vivo, ex vivo, or in vitro biologically active polypeptides of the invention to a tissue, population or cells, organ, graft, bodily system of a subject (e.g., organ system, lymphatic system, blood system, etc.); as DNA vaccines, multi-component vaccines for use in prophylactic or therapeutic treatment of a variety of diseases, disorders, or other conditions (e.g., cancers, viral-based disorders, angiogenic-based disorders) in a variety of subjects (e.g., mammals); as adjuvants to enhance or augment an immune response in a subject; as a component of a multiple-step boosting vaccination method (e.g., a format comprising a prime vaccination by delivery of a DNA or RNA nucleotide (e.g., a nucleotide encoding a polypeptide of the invention or encoding another polypeptide) followed by a second boost of a polypeptide (e.g., a polypeptide of the invention or other polypeptide); as diagnostic probes for the presence of complementary or partially complementary nucleic acids (including for detection of natural interferon-alpha coding nucleic acids); as substrates for further reactions, e.g., shuffling reactions, mutation reactions, or other diversity generation reactions to produce new and/or improved interferon-alpha homologues and new interferon-alpha nucleic acids encoding such homologues, e.g., to evolve novel therapeutic or prophylactic properties, and the like; for polymerase chain reactions (PCR) or cloning methods, e.g., including digestion or ligation reactions, to identify new and/or improved naturally-occurring or non-naturally occurring IFN-alpha nucleic acids and polypeptides encoded therefrom. Polynucleotides which encode an interferon homologue of the invention, or complements of the polynucleotides, are optionally administered to a cell to accomplish a therapeutically or prophylactically useful process or to express a therapeutically useful product in vivo, ex vivo, or in vitro. These applications, including in vivo or ex vivo applications, including, e.g., gene therapy, include a multitude of techniques by which gene expression may be altered in cells. Such methods include, for instance, the introduction of genes for expression of, e.g., therapeutically or prophylactically useful polypeptides, such as the interferon homologues of the present invention. Such methods include, for example, infecting with a retrovirus comprising the polynucleotides and/or polypeptides of the invention. Optionally, the retrovirus further comprises additional exogenous, e.g., therapeutic or prophylactic gene construct, sequences. In one aspect, the invention provides gene therapy methods of prophylactically or therapeutically treating a disease, disorder or condition in a subject in need of such treatment by administering in vivo, ex vivo, or in vitro one or more nucleic acids of the invention described herein to one or more cells of a subject, including an organism or mammal, including, e.g., a human, primate, mouse, pig, cow, goat, rabbit, rat, guinea pig, hamster, horse, sheep; or a non-mammalian vertebrate such as a bird (e.g., a chicken or duck) or a fish, or invertebrate, as described in more detail below.

In another aspect, the invention provides methods of prophylactically or therapeutically treating a disease, disorder or condition in a subject in need of such treatment by administering in vivo, ex vivo, or in vitro one or more polypeptides of the invention described herein to one or more cells of a subject (including those defined herein), as described in more detail below.

Polypeptide Expression

Polynucleotides encoding interferon homologue polypeptides of the invention are particularly useful for in vivo or ex vivo therapeutic or prophylactic applications, using techniques well known to those skilled in the art. For example, cultured cells are engineered ex vivo with a polynucleotide (DNA or RNA), with the engineered cells then being returned to the patient. Cells may also be engineered in vivo or ex vivo for expression of a polypeptide in vivo or ex vivo, respectively.

A number of viral vectors suitable for organismal in vivo or ex vivo transduction and expression are known. Such vectors include retroviral vectors (see Miller(1992) Curr. Top. Microbiol. Immunol. 158:1-24; Salmons and Gunzburg (1993) Human Gene Therapy 4:129-141; Miller et al. (1994) Methods in Enzymology 217:581-599) and adeno-associated vectors (reviewed in Carter (1992) Curr. Opinion Biotech. 3:533-539; Muzcyzka (1992) Curr. Top. Microbiol. Immunol. 158:97-129). Other viral vectors that are used include adenoviral vectors, herpes viral vectors and Sindbis viral vectors, as generally described in, e.g., Jolly (1994) Cancer Gene Therapy 1:51-64; Latchman (1994) Molec. Biotechnol. 2:179-195; and Johanning et al. (1995) Nucl. Acids Res. 23:1495-1501.

Gene therapy provides methods for combating chronic infectious diseases (e.g., HIV infection, viral hepatitis, Herpes Simplex Virus (HSV), hepatitis B (HepB), dengue virus, etc.), as well as non-infectious diseases including cancer and allergic diseases and some forms of congenital defects such as enzyme deficiencies. Several approaches for introducing nucleic acids into cells in vivo, ex vivo and in vitro have been used. These include liposome based gene delivery (Debs and Zhu (1993) WO 93/24640 and U.S. Pat. No. 5,641,662; Mannino and Gould-Fogerite (1988) BioTechniques 6(7):682-691; Rose, U.S. Pat No. 5,279,833; Brigham (1991) WO 91/06309; and Feigner et al. (1987) Proc. Nat'l Acad. Sci. USA 84:7413-7414); Brigham et al. (1989) Am. J. Med. Sci. 298:278-281; Nabel et al. (1990) Science 249:1285-1288; Hazinski et al. (1991) Am. J. Resp. Cell Molec. Biol. 4:206-209; and Wang and Huang (1987) Proc. Nat'l Acad. Sci. (USA) 84:7851-7855).; adenoviral vector mediated gene delivery, e.g., to treat cancer (see, e.g., Chen et al. (1994) Proc. Nat'l Acad. Sci. USA 91:3054-3057; Tong et al. (1996) Gynecol. Oncol. 61:175-179; Clayman et al. (1995) Cancer Res. 5:1-6; O'Malley et al. (1995) Cancer Res. 55:1080-1085; Hwang et al. (1995) Am. J. Respir. Cell Mol. Biol. 13:7-16; Haddada et al. (1995) Curr. Top. Microbiol. Immunol. 199 (Pt. 3):297-306; Addison et al. (1995) Proc. Nat'l Acad. Sci. USA 92:8522-8526; Colak et al. (1995) Brain Res. 691:76-82; Crystal (1995) Science 270:404-410; Elshami et al. (1996) Human Gene Ther. 7:141-148; Vincent et al. (1996) J. Neurosurg. 85:648-654), and many other diseases. Replication-defective retroviral vectors harboring therapeutic polynucleotide sequence as part of the retroviral genome have also been used, particularly with regard to simple MuLV vectors. See, e.g., Miller et al. (1990) Mol. Cell. Biol. 10:4239 (1990); Kolberg (1992) J. NIH Res. 4:43, and Cornetta et al. (1991) Hum. Gene Ther. 2:215). Nucleic acid transport coupled to ligand-specific, cation-based transport systems (Wu and Wu (1988) J. Biol. Chem. 263:14621-14624) have also been used. Naked DNA expression vectors have also been described (Nabel et al. (1990), supra); Wolff et al. (1990) Science 247:1465-1468). In general, these approaches can be adapted to the invention by incorporating nucleic acids encoding the interferon homologues herein into the appropriate vectors.

General texts which describe gene therapy protocols, which can be adapted to the present invention by introducing the nucleic acids of the invention into patients, include Robbins (1996) Gene Therapy Protocols, Humana Press, NJ, and Joyner (1993) Gene Targeting: A Practical Approach, IRL Press, Oxford, England.

Antisense Technology

In addition to expression of the nucleic acids of the invention as gene replacement nucleic acids, the nucleic acids are also useful for sense and anti-sense suppression of expression, e.g., to down-regulate expression of a nucleic acid of the invention, once expression of the nucleic acid is no-longer desired in the cell. Similarly, the nucleic acids of the invention, or subsequences or anti-sense sequences thereof, can also be used to block expression of naturally occurring homologous nucleic acids. A variety of sense and anti-sense technologies are known in the art, e.g., as set forth in Lichtenstein and Nellen (1997) Antisense Technology: A Practical Approach IRL Press at Oxford University, Oxford, England, and in Agrawal (1996) Antisense Therepeutics Humana Press, NJ, and the references cited therein.

Pharmaceutical Compositions

The polynucleotides and polypeptides of the invention (including vectors, cells, antibodies, etc., comprising polynucleotides or polypeptides of the invention) may be employed for therapeutic and prophylactic uses in combination with a suitable pharmaceutical carrier. Such compositions comprise a therapeutically or prophylactically effective amount of the polynucleotide or polypeptide of the invention, and a pharmaceutically acceptable carrier or excipient. A pharmaceutically acceptable carrier encompasses any of the standard pharmaceutical carriers, buffers and excipients. Such a carrier or excipient includes, but is not limited to, saline, buffered saline (e.g.,.phosphate-buffered saline solution), dextrose, water, glycerol, ethanol, emulsions (such as an oil/water or water/oil emulsion), various types of wetting agents and/or adjuvants, and combinations thereof. Suitable pharmaceutical carriers and agents are described in REMINGTON'S PHARMACEUTICAL SCIENCES (Mack Publishing Co., Easton, 19^{th} ed. 1995). The formulation should suit the mode of administration of the active agent (e.g., nucleotide, polypeptide, vector, cell, etc.). Methods of administering nucleic acids, polypeptides, vectors, cells, antibodies, and proteins are well known in the art, and further discussed below.

Use as Probes

Also contemplated are uses of polynucleotides, also referred to herein as oligonucleotides, typically having at least 12 bases, preferably at least 15, more preferably at least 20, 30, or 50 bases, which hybridize under at least highly stringent (or ultra-high stringent or ultra-ultra- high stringent conditions) conditions to an interferon homologue polynucleotide sequence described above. The polynucleotides may be used as probes, primers, sense and antisense agents, and the like, according to methods as noted supra.

### Sequence Variations

Silent Variations

It will be appreciated by those skilled in the art that due to the degeneracy of the genetic code, a multitude of nucleic acids sequences encoding interferon homologue polypeptides of the invention may be produced, some which may bear minimal sequence homology to the nucleic acid sequences explicitly disclosed herein.

TABLE 1

Codon Table

| | |
|---|---|
| Amino acids | Codon |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUG CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

For instance, inspection of the codon table (Table 1) shows that codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that U in an RNA sequence corresponds to T in a DNA sequence.

Using, as an example, the nucleic acid sequence corresponding to nucleotides 1-15 of SEQ ID NO:1, TGT GAT CTG CCT CAG, a silent variation of this sequence includes TGC GAC TTA CCA CAA, both sequences which encode the amino acid sequence CDLPQ, corresponding to amino acids 1-5 of SEQ ID NO:36.

Such “silent variations” are one species of “conservatively modified variations,” discussed below. One of skill will recognize that each codon in a nucleic acid (except AUG, which is ordinarily the only codon for methionine) can be modified by standard techniques to encode a functionally identical polypeptide. Accordingly, each silent variation of a nucleic acid which encodes a polypeptide is implicit in any described sequence. The invention provides each and every possible variation of nucleic acid sequence encoding a polypeptide of the invention that could be made by selecting combinations based on possible codon choices. These combinations are made in accordance with the standard triplet genetic code (e.g., as set forth in Table 1) as applied to the nucleic acid sequence encoding an interferon homologue polypeptide of the invention. All such variations of every nucleic acid herein are specifically provided and described by consideration of the sequence in combination with the genetic code.

Conservative Variations

“Conservatively modified variations” or, simply, “conservative variations” of a particular nucleic acid sequence refers to those nucleic acids which encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 3%, 2% or 1%) in an encoded sequence are “conservatively modified variations” where the alterations result in the deletion of an amino acid, addition of an amino acid, or substitution of an amino acid with a chemically similar amino acid.

Conservative substitution tables providing functionally similar amino acids are well known in the art. Table 2 sets forth six groups which contain amino acids that are “conservative substitutions” for one another.

TABLE 2
Conservative Substitution Groups

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

Thus, “conservatively substituted variations” or “conservative substitutions” of a listed polypeptide sequence of the present invention include substitutions of a small percentage, typically less than 5%, more typically less than 4%, 3%, 2% or 1%, of the amino acids of the polypeptide sequence, with a conservatively selected amino acid of the same conservative substitution group.

For example, a conservatively substituted variation of the polypeptide identified herein as SEQ ID NO:36 will contain “conservative substitutions”, according to the six groups defined above, in up to about 8 or 9 residues (i.e., about 5% of the amino acids) in the 166-amino acid polypeptide.

In a further example, if four conservative substitutions were localized in the region corresponding to amino acid residues 141-166 of SEQ ID NO:36, examples of conservatively substituted variations of this region,

WEVVR AEIMR SFSFS TNLQK RLRRKE include:

WEVVR SEIMR SFSYS TNLQR RLRRKD and

WELVR AEIVR SFSFS TNLNK RLRKKE and the like, in accordance with the conservative substitutions listed in Table 2 (in the above example, conservative substitutions are underlined). Listing of a protein sequence herein, in conjunction with the above substitution table, provides an express listing of all conservatively substituted proteins.

Finally, the addition of sequences which do not alter the encoded activity of a nucleic acid molecule, such as the addition of a non-functional sequence, is a conservative variation of the basic nucleic acid.

One of ordinary skill will appreciate that many conservative variations of the nucleic acid constructs which are disclosed yield a functionally identical construct. For example, as discussed above, owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence are substituted with different amino acids with highly similar properties, are also readily identified as being highly similar to a disclosed construct. Such conservative variations of each disclosed sequence are a feature of the present invention.

Nucleic Acid Hybridization

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes, part I, chapter 2, “Overview of principles of hybridization and the strategy of nucleic acid probe assays,” (Elsevier, N.Y.), as well as in Ausubel, supra, Hames and Higgins (1995) Gene Probes 1, IRL Press at Oxford University Press, Oxford, England (Hames and Higgins 1) and Hames and Higgins (1995) Gene Probes 2, IRL Press at Oxford University Press, Oxford, England (Hames and Higgins 2) provide details on the synthesis, labeling, detection and quantification of DNA and RNA, including oligonucleotides.

“Stringent hybridization wash conditions” in the context of nucleic acid hybridization experiments, such as Southern and northern hybridizations, are sequence dependent, and are different under different environmental parameters. An extensive guide to the hybridization of nucleic acids is found in Tijssen (1993), supra, and in Hames and Higgins 1 and Hames and Higgins 2, supra.

For purposes of the present invention, generally, “highly stringent” hybridization and wash conditions are selected to be about 5° C. or less lower lower than the thermal melting point (Tₘ) for the specific sequence at a defined ionic strength and pH (as noted below, highly stringent conditions can also be referred to in comparative terms). The Tₘ is the temperature (under defined ionic strength and pH) at which 50% of the test sequence hybridizes to a perfectly matched probe. Very stringent conditions are selected to be equal to the Tₘ for a particular probe.

The Tₘ is the temperature of the nucleic acid duplexes indicates the temperature at which the duplex is 50% denatured under the given conditions and its represents a direct measure of the stability of the nucleic acid hybrid. Thus, the Tₘ corresponds to the temperature corresponding to the midpoint in transition from helix to random coil; it depends on length, nucleotide composition, and ionic strength for long stretches of nucleotides.

After hybridization, unhybridized nucleic acid material can be removed by a series of washes, the stringency of which can be adjusted depending upon the desired results. Low stringency washing conditions (e.g., using higher salt and lower temperature) increase sensitivity, but can product nonspecific hybridization signals and high background signals. Higher stringency conditions (e.g., using lower salt and higher temperature that is closer to the hybridization temperature) lowers the background signal, typically with only the specific signal remaining. See Rapley, R. and Walker, J. M. eds., Molecular Biomethods Handbook (Humana Press, Inc. 1998) (hereinafter “Rapley and Walker”), which is incorporated herein by reference in its entirety for all purposes.

The Tₘ of a DNA-DNA duplex can be estimated using the following equation:

Tₘ (°C.)=81.5° C.+16.6 (log₁₀M)+0.41 (% G+C)−0.72 (% f)−500/n,

where M is the molarity of the monovalent cations (usually Na+), (% G+C) is the percentage of guanosine (G) and cystosine (C) nucleotides, (% f) is the percentage of formalize and n is the number of nucleotide bases (i.e., length) of the hybrid. See Rapley and Walker, supra.

The Tₘ of an RNA-DNA duplex can be estimated as follows:

Tₘ (°C.)=79.8° C.+18.5 (log₁₀M)+0.58 (% G+C)−11.8(% G+C)²−0.56(% f)−820/n,

where M is the molarity of the monovalent cations (usually Na+), (% G+C) is the percentage of guanosine (G) and cystosine (C) nucleotides, (% f) is the percentage of formamide and n is the number of nucleotide bases (i.e., length) of the hybrid. Id.

Equations 1 and 2 are typically accurate only for hybrid duplexes longer than about 100-200 nucleotides. Id.

The Tm of nucleic acid sequences shorter than 50 nucleotides can be calculated as follows:

Tₘ (° C.)=4(G+C)+2(A+T),

where A (adenine), C, T (thymine), and G are the numbers of the corresponding nucleotides.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of stringent wash conditions is a 0.2×SSC wash at 65° C. for 15 minutes (see Sambrook, supra for a description of SSC buffer). Often the high stringency wash is preceded by a low stringency wash to remove background probe signal. An example low stringency wash is 2×SSC at 40° C. for 15 minutes.

In general, a signal to noise ratio of 2.5×-5× (or higher) than that observed for an unrelated probe in the particular hybridization assay indicates detection of a specific hybridization. Detection of at least stringent hybridization between two sequences in the context of the present invention indicates relatively strong structural similarity or homology to, e.g., the nucleic acids of the present invention provided in the sequence listings herein.

As noted, “highly stringent” conditions are selected to be about 5° C. or less lower than the thermal melting point (Tₘ) for the specific sequence at a defined ionic strength and pH. Target sequences that are closely related or identical to the nucleotide sequence of interest (e.g., “probe”) can be identified under highly stringency conditions. Lower stringency conditions are appropriate for sequences that are less complementary. See, e.g., Rapley and Walker, supra.

Comparative hybridization can be used to identify nucleic acids of the invention, and this comparative hybridization method is a preferred method of distinguishing nucleic acids of the invention. Detection of highly stringent hybridization between two nucleotide sequences in the context of the present invention indicates relatively strong structural similarity/homology to, e.g., the nucleic acids provided in the sequence listing herein. Highly stringent hybridization between two nucleotide sequences demonstrates a degree of similarity or homology of structure, nucleotide base composition, arrangement or order that is greater than that detected by stringent hybridization conditions. In particular, detection of highly stringent hybridization in the context of the present invention indicates strong structural similarity or structural homology (e.g., nucleotide structure, base composition, arrangement or order) to, e.g., the nucleic acids provided in the sequence listings herein. For example, it is desirable to identify test nucleic acids which hybridize to the exemplar nucleic acids herein under stringent conditions.

Thus, one measure of stringent hybridization is the ability to hybridize to one of the listed nucleic acids (e.g., nucleic acid sequences SEQ ID NO:1 to SEQ ID NO:35, and SEQ ID NO:72 to SEQ ID NO:78, and complementary polynucleotide sequences thereof) under highly stringent conditions (or very stringent conditions, or ultra-high stringency hybridization conditions, or ultra-ultra high stringency hybridization conditions). Stringent hybridization (including, e.g., highly stringent, ultra-high stringency, or ultra-ultra high stringency hybridization conditions) and wash conditions can easily be determined empirically for any test nucleic acid.

For example, in determining highly stringent hybridization and wash conditions, the hybridization and wash conditions are gradually increased (e.g., by increasing temperature, decreasing salt concentration, increasing detergent concentration and/or increasing the concentration of organic solvents, such as formalin, in the hybridization or wash), until a selected set of criteria are met. For example, the hybridization and wash conditions are gradually increased until a probe comprising one or more nucleic acid sequences selected from SEQ ID NO:1 to SEQ ID NO:35, SEQ ID NO:72 to SEQ ID NO:78, and complementary polynucleotide sequences thereof, binds to a perfectly matched complementary target (again, a nucleic acid comprising one or more nucleic acid sequences selected from SEQ ID NO:1 to SEQ ID NO:35, SEQ ID NO:72 to SEQ ID NO:78, and complementary polynucleotide sequences thereof), with a signal to noise ratio that is at least 2.5×, and optionally 5× or more as high as that observed for hybridization of the probe to an unmatched target. In this case, the unmatched target is a nucleic acid corresponding to a known alpha interferon, e.g., an alpha interferon nucleic acid that is present in a public database such as GenBank™ at the time of filing of the subject application. Examples of such unmatched target nucleic acids include, e.g., those with the following GenBank accession numbers: J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1). Additional such sequences can be identified in GenBank by one of ordinary skill in the art. Nomenclature of the human interferon genes and proteins is discussed in Diaz et al., (1996) J. Interferon and Cytokine Res. 16:179-180 and Allen et al. (1996) J. Interferon and Cytokine Res. 16:181-184, respectively, each of which is incorporated herein by reference in its entirety for all purposes.

A test nucleic acid is said to specifically hybridize to a probe nucleic acid when it hybridizes at least ½ as well to the probe as to the perfectly matched complementary target, i.e., with a signal to noise ratio at least ½ as high as hybridization of the probe to the target under conditions in which the perfectly matched probe binds to the perfectly matched complementary target with a signal to noise ratio that is at least about 2.5×-10×, typically 5×-10× as high as that observed for hybridization to any of the unmatched target nucleic acids represented by GenBank accession numbers J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1), or other similar interferon-alpha sequences presented in GenBank.

Ultra high-stringency hybridization and wash conditions are those in which the stringency of hybridization and wash conditions are increased until the signal to noise ratio for binding of the probe to the perfectly matched complementary target nucleic acid is at least 10× as high as that observed for hybridization to any of the unmatched target nucleic acids represented by GenBank accession numbers J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1), or other similar IFN-alpha sequences presented in GenBank A target nucleic acid which hybridizes to a probe under such conditions, with a signal to noise ratio of at least ½ that of the perfectly matched complementary target nucleic acid is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or 500× or more as high as that observed for hybridization to any of the unmatched target nucleic acids represented by GenBank accession numbers J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289,V00549 (alpha-2a), and I08313 (alpha-Con1),or other similar interferon-alpha sequences presented in GenBank, can be identified. A target nucleic acid which hybridizes to a probe under such conditions, with a signal to noise ratio of at least ½ that of the perfectly matched complementary target nucleic acid is said to bind to the probe under ultra-ultra-high stringency conditions.

Target nucleic acids which hybridize to the nucleic acids represented by SEQ ID NO:1 to SEQ ID NO:35 and SEQ ID NO:72 to SEQ ID NO:78 under high, ultra-high and ultra-ultra high stringency conditions are a feature of the invention. Examples of such nucleic acids include those with one or a few silent or conservative nucleic acid substitutions as compared to a given nucleic acid sequence.

Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code, or when antisera or antiserum generated against one or more of SEQ ID NO:36 to SEQ ID NO:70 and SEQ ID NO:79 to SEQ ID NO:85 which has been subtracted using the polypeptides encoded by known interferon-alpha sequences, including, e.g., the those encoded by the following interferon-alpha nucleic acid sequences in GenBank: Accession numbers J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con 1), or other similar interferon-alpha sequences presented in GenBank. Further details on immunological identification of polypeptides of the invention are found below. Additionally, for distinguishing between duplexes with sequences of less than about 100 nucleotides, a TMAC1 hybridization procedure known to those of ordinary skill in the art can be used. See, e.g., Sorg, U. et al. 1 Nucleic Acids Res. (Sep. 11, 1991) 19(17), incorporated herein by reference in its entirety for all purposes.

In one aspect, the invention provides a nucleic acid which comprises a unique subsequence in a nucleic acid selected from SEQ ID NO:1 to SEQ ID NO:35 or SEQ ID NO:72 to SEQ ID NO:78. The unique subsequence is unique as compared to a nucleic acid corresponding to any known interferon-alpha nucleic acid sequence including, e.g., the known sequences represented by GenBank accession numbers J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), A12109, R0067 (Gx-1), I01614, I01787, I07821, M12350 (alpha-F), M38289, V00549 (alpha-2a), and I08313 (alpha-Con1), or other similar interferon-alpha sequences presented in GenBank. Such unique subsequences can be determined by aligning any of SEQ ID NO:1 to SEQ ID NO:35 or SEQ ID NO:72 to SEQ ID NO:78 against the complete set of nucleic acids corresponding to GenBank accession numbers of known interferon-alpha nucleic acid sequences, such as, e.g., J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), A12109 (alpha-4B), R0067 (Gx-1), I01614, I01787, I07821, M12350 (alpha-F), M38289,V00549 (alpha-2a), and I08313 (alpha-Con 1), or other similar interferon-alpha sequences presented in GenBank. Alignment can be performed using the BLAST algorithm set to default parameters. Any unique subsequence is useful, e.g., as a probe to identify the nucleic acids of the invention.

Similarly, the invention includes a polypeptide which comprises a unique amino acid subsequence in a polypeptide selected from: SEQ ID NO:36 to SEQ ID NO:70 or SEQ ID NO:79 to SEQ ID NO:85. Here, the unique subsequence is unique as compared to an amino acid subsequence of a known interferon-alpha polypeptide including, e.g., an amino acid subsequence of a polypeptide encoded by a known interferon-alpha nucleic acid corresponding to any of GenBank accession numbers: J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1), or other similar interferon-alpha nucleic acid or polypeptide sequences presented in GenBank. Here again, the polypeptide is aligned against the complete set of known interferon-alpha polypeptide sequences, such as those polypeptides encoded by nucleic acids corresponding to GenBank accession numbers J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), - M38289, V00549 (alpha-2a), and I08313 (alpha-Con1), (referred to as “the control polypeptides”) (note that where the sequence corresponds to a non-translated sequence such as a pseudo gene, the corresponding polypeptide is generated simply by in silico translation of the nucleic acid sequence into an amino acid sequence, where the reading frame is selected to correspond to the reading frame of homologous alpha interferon nucleic acids) or other similar interferon-alpha nucleic acid or polypeptide sequences presented in GenBank.

In addition, the present invention provides a target nucleic acid which hybridizes under at least stringent or highly stringent conditions (or conditions of greater stringency) to a unique coding oligonucleotide which encodes a unique subsequence in a polypeptide selected from: SEQ ID NO:36 to SEQ ID NO:70 or SEQ ID NO:79 to SEQ ID NO:85, wherein the unique subsequence is unique as compared to a an amino acid subsequence of a known interferon-alpha polypeptide sequence shown in GenBank or to a polypeptide corresponding to any of the control polypeptides. Unique sequences are determined as noted above.

In one example, the stringent conditions are selected such that a perfectly complementary oligonucleotide to the coding oligonucleotide hybridizes to the coding oligonucleotide with at least about a 5-10× higher signal to noise ratio than for hybridization of the perfectly complementary oligonucleotide to a control nucleic acid corresponding to any of the control polypeptides. Conditions can be selected such that higher ratios of signal to noise are observed in the particular assay which is used, e.g., about 15×, 20×, 30×, 50× or more. In this example, the target nucleic acid hybridizes to the unique coding oligonucleotide with at least a 2× higher signal to noise ratio as compared to hybridization of the control nucleic acid to the coding oligonucleotide. Again, higher signal to noise ratios can be selected, e.g., about 2.5×, about 5×, about 10×, about 20×, about 30×, about 50× or more. The particular signal will depend on the label used in the relevant assay, e.g., a fluorescent label, a calorimetric label, a radio active label, or the like.

In another aspect, the invention provides a polypeptide that comprises unique subsequence in a polypeptide selected from SEQ ID NO:36 to SEQ ID NO:70 and SEQ ID NO:79 to SEQ ID NO:85, wherein the unique subsequence is unique as compared to a polypeptide sequence corresponding to a known interferon-alpha polypeptide, such as, e.g., an interferon-alpha polypeptide sequence present in GenBank.

### Substrates and Formats for Sequence Recombination

The polynucleotides of the invention are useful as substrates for a variety of recombination and recursive recombination (e.g., DNA shuffling) reactions, as well as other diversity generating techniques, including mutagenesis techniques and standard cloning methods as set forth in, e.g., Ausubel, Berger and Sambrook, supra, i.e., to produce additional interferon-alpha homologues with desired properties. Based on the screening or selection protocols employed, recombinant, e.g., shuffled, interferon-alpha homologue polypeptides can be generated and isolated that confer a variety of desirable characteristics, e.g., enhanced antiviral activity, enhanced antiproliferative activity, increased growth inhibitory, cytostatic and/or cytotoxic activities towards particular target cells, reduced immunogenicity, etc.

A variety of diversity generating protocols, including nucleic acid shuffling protocols, are available and fully described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids that encode proteins with or which confer desirable properties. Following diversification by one or more of the methods herein, or otherwise available to one of skill, any nucleic acids that are produced can be selected for a desired activity or property, e.g., enhanced antiviral activity, enhanced antiproliferative activity, enhanced anti-angiogenic activity, increased growth inhibitory, cytostatic and/or cytotoxic activities towards particular target cells, reduced immunogenicity, etc. Methods for determining nucleic acids having enhanced antiviral, antiproliferative, growth inhibitory, cytostatic, and/or cytotoxic activity or reduced immunogenicity include those described herein. This can include identifying any activity that can be detected, for example, in an automated or automatable format, by any of the assays in the art. A variety of related (or even unrelated) properties can be evaluated, in serial or in parallel, at the discretion of the practitioner.

The following publications describe a variety of diversity generating procedures, including recursive recombination procedures, and/or methods for generating modified nucleic acid sequences for use in the procedures and methods of the present invention include the following publications and the references cited therein: Soong, N. W. et al. (2000) “Molecular Breeding of Viruses,” Nature Genetics 25:436-439; Stemmer, W. et al. (1999) “Molecular breeding of viruses for targeting and other clinical properties,” Tumor Targeting 4:1-4; Ness et al. (1999) “DNA Shuffling of subgenomic sequences of subtilisin,” Nature Biotechnology 17:893-896; Chang et al. (1999) “Evolution of a cytokine using DNA family shuffling,” Nature Biotechnology 17:793-797; Minshull and Stemmer (1999) “Protein evolution by molecular breeding,” Current Opinion in Chemical Biology 3:284-290; Christians et al. (1999) “Directed evolution of thymidine kinase for AZT phosphorylation using DNA family shuffling,” Nature Biotechnology 17:259-264; Crameri et al. (1998) “DNA shuffling of a family of genes from diverse species accelerates directed evolution,” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening,” Proc. Nat'l Acad. Sci. USA 94:4504-4509; Patten et al. (1997) “Applications of DNA Shuffling to Pharmaceuticals and Vaccines,” Current Opinion in Biotechnology 8:724-733; Crameri et al. (1996) “Construction and evolution of antibody-phage libraries by DNA shuffling,” Nature Medicine 2:100-103; Crameri et al. (1996) “Improved green fluorescent protein by molecular evolution using DNA shuffling,” Nature Biotechnology 14:315-319; Gates et al. (1996) “Affinity selective isolation of ligands from peptide libraries through display on a lac repressor ‘headpiece dimer,’” J. Mol. Biol. 255:373-386; Stemmer (1996) “Sexual PCR and Assembly PCR” In: The Encyclopedia of Molecular Biology, VCH Publishers, New York. pp. 447-457; Crameri and Stemmer (1995) “Combinatorial multiple cassette mutagenesis creates all the permutations of mutant and wildtype cassettes,” BioTechniques 18:194-195; Stemmer et al. (1995) “Single-step assembly of a gene and entire plasmid form large numbers of oligodeoxy-ribonucleotides” Gene 164:49-53; Stemmer (1995) “The Evolution of Molecular Computation,” Science 270:1510; Stemmer (1995) “Searching Sequence Space,” Bio/Technology 13:549-553; Stemmer (1994) “Rapid evolution of a protein in vitro by DNA shuffling,” Nature 370:389-391; and Stemmer (1994) “DNA shuffling by random fragmentation and reassembly: In vitro recombination for molecular evolution,” Proc. Nat'l Acad. Sci. USA 91:10747-10751.

Additional details regarding DNA shuffling and other diversity generating methods can be found in the following U.S. patents, PCT publications, and EP publications: U.S. Pat. No. 5,605,793 to Stemmer (Feb. 25, 1997), “Methods for In vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer (Nov. 10, 1998) “End-Complementary Polymerase Reaction;” U.S. Pat. No. 5,837,458 to Minshull (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz, “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al., “Antigen Library Immunization;” WO 99/41369 by Punnonen et al., “Genetic Vaccine Vector Engineering;” WO 99/41368 by Punnonen et al., “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by Del Cardayre et al. “Evolution of Whole Cells and Organisms by Recursive Sequence Recombination;” WO 98/27230 by Patten and Stemmer, “Methods and Compositions for Polypeptide Engineering;” EP 0946755 by Patten and Stemmer, “Methods and Compositions for Polypeptide Engineering;” and WO 98/13487 by Stemmer et al., “Methods for Optimization of Gene Therapy by Recursive Sequence Shuffling and Selection;” WO 00/00632, “Methods for Generating Highly Diverse Libraries,” WO 00/09679, “Methods for Obtaining in vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Certain U.S. applications provide additional details regarding DNA shuffling and related techniques, as well as other diversity generating methods, including “SHUFFLING OF CODON ALTERED GENES” by Patten et al. filed Sep. 29, 1998 (U.S. Ser. No. 60/102,362), Jan. 29, 1999 (U.S. Ser. No. 60/117,729), and Sep. 28, 1999 (U.S. Ser. No. 09/407,800); “EVOLUTION OF WHOLE CELLS AND ORGANISMS BY RECURSIVE SEQUENCE RECOMBINATION”, by Del Cardayre et al. filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Feb. 5, 1999 (U.S. Ser. No. 60/118,813), Jun. 24, 1999 (U.S. Ser. No. 60/141,049), and Sep. 28, 1999 (U.S. Ser. No. 09/408,392); “USE OF CODON-BASED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov and Stemmer, filed Feb. 5, 1999 (U.S. Ser. No. 60/118854) and Oct. 12, 1999 (U.S. Ser. No. 09/416,375); RECOMBINATION OF INSERTION MODIFIED NUCLEIC ACIDS by Patten et al., filed Mar. 5, 1999 (U.S. Ser. No. 60/122,943), Jul. 2, 1999 (U.S. Ser. No. 60/142,299), Nov. 10, 1999 (U.S. Ser. No. 60/164,618), and Nov. 10, 1999 (U.S. Ser. No. 60/164,617); and “SINGLE-STRANDED NUCLEIC ACID TEMPLATE-MEDIATED RECOMBINATION AND NUCLEIC ACID FRAGMENT ISOLATION” by Affholter, U.S. Ser. No. 60/186,482 filed Mar. 2, 2000.

As a review of the foregoing publications, patents, published foreign applications and U.S. patent applications reveals, diversity generation methods, such as shuffling (or “recursive recombination”) of nucleic acids, to provide new nucleic acids with desired properties can be carried out by a number of established methods. Any of these methods can be adapted to the present invention to evolve the alpha interferons discussed herein to produce new alpha interferon homologues with new or improved properties. Both the methods of making such interferons and the interferons (e.g., IFN homologues) produced by these methods are a feature of the invention. In brief, several different general classes of sequence modification methods, such as recombination, are applicable to the present invention and set forth, e.g., in the references above. First, nucleic acids can be recombined in vitro by any of a variety of techniques discussed in the references above, including e.g., DNAse digestion of nucleic acids to be recombined followed by ligation and/or PCR reassembly of the nucleic acids. Second, nucleic acids can be recursively recombined in vivo or ex vivo, e.g., by allowing recombination to occur between nucleic acids in cells. Third, whole genome recombination methods can be used in which whole genomes of cells or other organisms are recombined, optionally including spiking of the genomic recombination mixtures with desired library components (e.g., genes corresponding to the pathways of the present invention). Fourth, synthetic recombination methods can be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Fifth, in silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/ gene reassembly techniques. Any of the preceding general recombination formats can be practiced in a reiterative fashion to generate a more diverse set of recombinant nucleic acids. Sixth, methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be used. above references provide these and other basic recombination formats as well as many modifications of these formats. Regardless of the format which is used, the nucleic acids of the invention can be recombined (with each other, or with related (or even unrelated) nucleic acids to produce a diverse set of recombinant nucleic acids, including e.g., homologous nucleic acids. In general, the sequence recombination techniques described herein provide particular advantages in that they provide for recombination between the nucleic acids of SEQ ID NO:1 to SEQ ID NO:35, and SEQ ID NO:72 to SEQ ID NO:78, or fragments or variants thereof, in any available format, thereby providing a very fast way of exploring the manner in which different combinations of sequences can affect a desired result.

Following recombination, any nucleic acids which are produced can be screened or selected for a desired activity. In the context of the present invention, this can include testing for and identifying any activity that can be detected, e.g., in an automatable format, by any assay known in the art. In addition, useful properties such as low immunogenicity, increased half-life, improved solubility, oral availability, or the like can also be selected for. A variety of alpha-interferon related (or even unrelated) properties can be assayed for, using any available assay.

DNA mutagenesis and shuffling provide a robust, widely applicable, means of generating diversity useful for the engineering of proteins, pathways, cells and organisms with improved characteristics. In addition to the basic formats described above, it is sometimes desirable to combine shuffling methodologies with other techniques for generating diversity. In conjunction with (or separately from) shuffling methods, a variety of diversity generation methods can be practiced and the results (i.e., diverse populations of nucleic acids) screened for in the systems of the invention. Additional diversity can be introduced by methods which result in the alteration of individual nucleotides or groups of contiguous or non-contiguous nucleotides, i.e., mutagenesis methods. Many mutagenesis methods are found in the above-cited references; additional details regarding mutagenesis methods can be found in the references listed below.

Mutagenesis methods of generating diversity include, for example, recombination (PCT/US98/05223; Publ. No. WO98/42727); site-directed mutagenesis (Ling et al. (1997) “Approaches to DNA mutagenesis: an overview,” Anal. Biochem. 254(2):157-178; Dale et al. (1996) “Oligonucleotide-directed random mutagenesis using the phosphorothioate method,” Methods Mol. Biol. 57:369-374; Smith (1985) “In vitro mutagenesis,” Ann. Rev. Genet. 19:423-462; Botstein & Shortle (1985) “Strategies and applications of in vitro mutagenesis,” Science 229:1193-1201; Carter (1986) “Site-directed mutagenesis,” Biochem. J. 237:1-7; and Kunkel (1987) “The efficiency of oligonucleotide directed mutagenesis,” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection,” Proc. Nat'l Acad. Sci. USA 82:488-492; Kunkel et al. (1987) “Rapid and efficient site-specific mutagenesis without phenotypic selection,” Results Probl. Cell Differ. 154, 367-382; and Bass et al. (1988) “Mutant Trp repressors with new DNA-binding specificities,” Science 242:240-245); oligonucleotide-directed mutagenesis (Results Probl. Cell Differ. 100:468-500 (1983); Results Probl. Cell Differ. 154:329-350 (1987); Zoller & Smith (1982) “Oligonucleotide-directed mutagenesis using M13-derived vectors: an efficient and general procedure for the production of point mutations in any DNA fragment,” Nucleic Acids Res. 10:6487-6500; Zoller & Smith (1983) “Oligonucleotide-directed mutagenesis of DNA fragments cloned into M13 vectors,” Results Probl. Cell Differ. 100:468-500; and Zoller & Smith (1987) “Oligonucleotide-directed mutagenesis: a simple method using two oligonucleotide primers and a single-stranded DNA template,” Results Probl. Cell Differ. 154:329-350); phosphorothioate-modified DNA mutagenesis (Taylor et al. (1985) “The use of phosphorothioate-modified DNA in restriction enzyme reactions to prepare nicked DNA,” Nucl. Acids Res. 13:8749-8764; Taylor et al. (1985) “The rapid generation of oligonucleotide-directed mutations at high frequency using phosphorothioate-modified DNA,” Nucl. Acids Res. 13:8765-8787 (1985); Nakamaye & Eckstein (1986) “Inhibition of restriction endonuclease Nci I cleavage by phosphorothioate groups and its application to oligonucleotide-directed mutagenesis,” Nucl. Acids Res. 14:9679-9698; Sayers et al. (1988) “Y-T Exonucleases in phosphorothioate-based oligonucleotide-directed mutagenesis,” Nucl. Acids Res. 16:791-802; and Sayers et al. (1988) “Strand specific cleavage of phosphorothioate-containing DNA by reaction with restriction endonucleases in the presence of ethidium bromide,” Nucl. Acids Res. 16:803-814); mutagenesis using gapped duplex DNA (Kramer et al. (1984) “The gapped duplex DNA approach to oligonucleotide-directed mutation construction,” Nucl. Acids Res. 12:9441-9456; Kramer & Fritz (1987) “Oligonucleotide-directed construction of mutations via gapped duplex DNA,” Results Probl. Cell Differ. 154:350-367; Kramer et al. (1988) “Improved enzymatic ii vitro reactions in the gapped duplex DNA approach to oligonucleotide-directed construction of mutations,” Nucl. Acids Res. 16:7207; and Fritz et al. (1988) “Oligonucleotide-directed construction of mutations: a gapped duplex DNA procedure without enzymatic reactions in vitro,” Nucl. Acids Res. 16:6987-6999).

Additional suitable methods include point mismatch repair (Kramer et al. (1984) “Point Mismatch Repair,” Cell 38:879-887), mutagenesis using repair-deficient host strains (Carter et al. (1985) “Improved oligonucleotide site-directed mutagenesis using M13 vectors,” Nucl. Acids Res. 13:4431-4443; and Carter (1987) “Improved oligonucleotide-directed mutagenesis using M13 vectors,” Results Probl. Cell Differ. 154:382-403), deletion mutagenesis (Eghtedarzadeh & Henikoff (1986) “Use of oligonucleotides to generate large deletions,” Nucl. Acids Res. 14:5115), restriction-selection and restriction-selection and restriction-purification (Wells et al. (1986) “Importance of hydrogen-bond formation in stabilizing the transition state of subtilisin,” Phil. Trans. R. Soc. Lond. A 317:415-423), mutagenesis by total gene synthesis (Nambiar et al. (1984) “Total synthesis and cloning of a gene coding for the ribonuclease S protein,” Science 223:1299-1301; Sakamar and Khorana (1988) “Total synthesis and expression of a gene for the a-subunit of bovine rod outer segment guanine nucleotide-binding protein (transducing),” Nucl. Acids Res. 14:6361-6372; Wells et al. (1985) “Cassette mutagenesis: an efficient method for generation of multiple mutations at defined sites,” Gene 34:315-323; and Grundström et al. (1985) “Oligonucleotide-directed mutagenesis by microscale ‘shot-gun’ gene synthesis,” Nucl. Acids Res. 13:3305-3316), double-strand break repair (Mandecki (1986) “Oligonucleotide-directed double-strand break repair in plasmids of Escherichia coli: a method for site-specific mutagenesis,” Proc. Nat'l Acad. Sci. USA, 83:7177-7181). Additional details on many of the above methods can be found in Methods in Enzymology, Vol. 154, which also describes useful controls for trouble-shooting problems with various mutagenesis methods.

Random or semi-random mutagenesis using doped or degenerate oligonucleotides (Arkin and Youvan (1992) “Optimizing nucleotide mixtures to encode specific subsets of amino acids for semi-random mutagenesis,” Biotechnology 10:297-300; Reidhaar-Olson et al. (1991) “Random mutagenesis of protein sequences using oligonucleotide cassettes,” Methods Enzymol. 208:564-86; Lim and Sauer (1991) “The role of internal packing interactions in determining the structure and stability of a protein,” J. Mol. Biol. 219:359-76; Breyer and Sauer (1989) “Mutational analysis of the fine specificity of binding of monoclonal antibody 51F to lambda repressor,” J. Biol. Chem. 264:13355-60); “Walk-Through Mutagenesis” (Crea, R.; U.S. Pat. Nos. 5,830,650 and 5,798,208, and EP Patent 0527809 B1) may also be employed to generate diversity.

In one aspect of the present invention, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same vial, with the products of one reaction priming the products of another reaction. Sexual PCR mutagenesis can be used in which homologous recombination occurs between DNA molecules of different but related DNA sequence ill vitro, by random fragmentation of the DNA molecule based on sequence homology, followed by fixation of the crossover by primer extension in a PCR reaction. This process is described in the references above, e.g., in Stemmer (1994) Proc. Nat'l Acad. Sci. USA 91:10747-10751. Recursive ensemble mutagenesis can be used in which an algorithm for protein mutagenesis is used to produce diverse populations of phenotypically related mutants whose members differ in amino acid sequence. This method uses a feedback mechanism to control successive rounds of combinatorial cassette mutagenesis. Examples of this approach are found in Arkin & Youvan (1992) Proc. Nat'l Acad. Sci. USA 89:7811-7815.

As noted, oligonucleotide directed mutagenesis can be used in a process which allows for the generation of site-specific mutations in any nucleic acid sequence of interest. Examples of such techniques are found in the references above and, e.g., in Reidhaar-Olson et al. (1988) Science, 241:53-57. Similarly, cassette mutagenesis can be used in a process which replaces a small region of a double stranded DNA molecule with a synthetic oligonucleotide cassette that differs from the native sequence. The oligonucleotide can contain, e.g., completely and/or partially randomized native sequence(s).

In vivo (or ex vivo) mutagenesis can be used in a process of generating random mutations in any cloned DNA of interest which involves the propagation of the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA.

Exponential ensemble mutagenesis can be used for generating combinatorial libraries with a high percentage of unique and functional mutants, where small groups of residues are randomized in parallel to identify, at each altered position, amino acids which lead to functional proteins. Examples of such procedures are found in Delegrave & Youvan (1993) Biotechnology Research 11: 1548-1552. Similarly, random and site-directed mutagenesis can be used. Examples of such procedures are found in Arnold (1993) Current Opinion in Biotechnology 4:450-455.

Kits for mutagenesis, library construction, and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc., Lemargo Inc., Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd. (e.g., using the Carter/Winter method above).

Any of the described shuffling or mutagenesis techniques can be used in conjunction with procedures which introduce additional diversity into a genome, e.g., a bacterial, fungal, animal or plant genome. For example, in addition to the methods above, techniques have been proposed which produce chimeric nucleic acid multimers suitable for transformation into a variety of species (see, e.g., Schellenberger U.S. Pat. No. 5,756,316 and the references above). When such chimeric multimers consist of genes that are divergent with respect to one another (e.g., derived from natural diversity or through application of site directed mutagenesis, error prone PCR, passage through mutagenic bacterial strains, and the like), are transformed into a suitable host, this provides a source of nucleic acid diversity for DNA diversification.

Chimeric multimers transformed into host species are suitable as substrates for in vivo (or ex vivo) shuffling protocols. Alternatively, a multiplicity of polynucleotides sharing regions of partial sequence similarity or homology can be transformed into a host species and recombined in vivo (or ex vivo) by the host cell. Subsequent rounds of cell division can be used to generate libraries, members of which, comprise a single, homogenous population of monomeric or pooled nucleic acid. Alternatively, the monomeric nucleic acid can be recovered by standard techniques and recursively recombined in any of the described shuffling formats.

Chain termination methods of diversity generation have also been proposed (see, e.g., U.S. Pat. No. 5,965,408 and the references above). In this approach, double stranded DNAs corresponding to one or more genes sharing regions of sequence similarity or homology are combined and denature, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity or homology and which are chimeric with respect to the starting population of DNA molecules. Optionally, the products or partial pools of the products can be amplified at one or more stages in the process. Polynucleotides produced by a chain termination method, such as described above are suitable substrates for diversity generation methods (e.g., RSR, DNA shuffling) according to any of the described formats.

Diversity can be further increased by using methods which are not homology based with DNA shuffling (which, as set forth in the above publications and applications can be homology or non-homology based, depending on the precise format). For example, incremental truncation for the creation of hybrid enzymes (ITCHY) described in Ostermeier et al. (1999) “A combinatorial approach to hybrid enzymes independent of DNA homology” Nature Biotech. 17:1205, can be used to generate an initial recombinant library which serves as a substrate for one or more rounds of in vitro, ex vivo, or in vivo diversity generation methods (e.g., RSR or shuffling methods).

Methods for generating multispecies expression libraries have been described (e.g., U.S. Pat. Nos. 5,783,431; 5,824,485 and the references above) and their use to identify protein activities of interest has been proposed (U.S. Pat. No. 5,958,672 and the references above). Multispecies expression libraries are, in general, libraries comprising cDNA or genomic sequences from a plurality of species or strains, operably linked to appropriate regulatory sequences, in an expression cassette. The cDNA and/or genomic sequences are optionally randomly concatenated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

In some applications, it is desirable to preselect or prescreen libraries (e.g., an amplified library, a genomic library, a cDNA library, a normalized library, etc.) or other substrate nucleic acids prior to shuffling, or to otherwise bias the substrates towards nucleic acids that encode functional products (shuffling procedures can also, independently have these effects). For example, in the case of antibody engineering, it is possible to bias the shuffling process toward antibodies with functional antigen binding sites by taking advantage of in vivo (or en vivo or in vitro) recombination events prior to diversity generation (e.g., DNA shuffling) by any described method. For example, recombined CDRs derived from B cell cDNA libraries can be amplified and assembled into framework regions (e.g., Jirholt et al. (1998) “Exploiting sequence space: shuffling in vivo formed complementarity determining regions into a master framework,” Gene 215:471) prior to diversity generation (e.g., DNA shuffling) according to any of the methods described herein.

Libraries can be biased towards nucleic acids which encode proteins with desirable activities (e.g., binding affinities, enzymatic activities, anti-viral activities, ability to induce an immune response, antiproliferative activities, adjuvant properties, etc.). For example, after identifying a clone from a library which exhibits a specified activity, the clone can be mutagenized using any known method for introducing DNA alterations, including, but not restricted to, DNA shuffling or another form of recursive sequence recombination or diversity generation. A library comprising the mutagenized homologues is then screened for a desired activity, which can be the same as or different from the initially specified activity. An example of such a procedure is proposed in U.S. Pat. No. 5,939,250. Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom.

Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in a shuffling-based gene reassembly process. In one such method the fragment population derived the genomic library(ies) is annealed with partial, or, often approximately full length ssDNA or RNA corresponding to the opposite strand. Assembly of complex chimeric genes from this population is the mediated by nuclease-base removal of non-hybridizing fragment ends, polymerization to fill gaps between such fragments and subsequent single stranded ligation. The parental strand can be removed by digestion (if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. As set forth in “Single-stranded nucleic acid template-mediated recombination and nucleic acid fragment isolation” by Affholter (U.S. Ser. No. 60/186,482, filed Mar. 2, 2000) and WO 98/27230, “Methods and Compositions for Polypeptide Engineering” by Patten and Stemmer, shuffling using single-stranded templates and nucleic acids of interest which bind to a portion of the template can also be performed.

In one approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for any of the shuffling reactions described herein.

“Non-Stochastic” methods of generating nucleic acids and polypeptides are alleged in Short, J. “Non-Stochastic Generation of Genetic Vaccines and Enzymes,” WO 00/46344. These methods, including the proposed non-stochastic polynucleotide reassembly and gene site saturation mutagenesis and synthetic ligation polynucleotide reassembly methods outlined therein, can be applied to the present invention as well.

It will readily be appreciated that any of the above described techniques suitable for enriching a library prior to diversification can also be used to screen the products, or libraries of products, produced by the diversity generating methods.

A recombinant nucleic acid produced by recursively recombining one or more polynucleotides of the invention with one or more additional nucleic acids also forms a part of the invention. The one or more additional nucleic acids may include another polynucleotide of the invention; optionally, alternatively, or in addition, the one or more additional nucleic acids can include, e.g., a nucleic acid encoding a naturally-occurring interferon-alpha or a subsequence thereof, or any homologous interferon-alpha sequence or subsequence thereof, or an interferon-beta sequence or subsequence thereof (e.g., an interferon-alpha or interferon-beta sequence as found in GenBank or other available literature), or, e.g., any other homologous or non-homologous nucleic acid (certain recombination formats noted above, notably those performed synthetically or in silico, do not require homology for recombination).

The recombining steps may be performed in vivo, ex vivo, in vitro, or in silico as described in more detail in the references above. Also included in the invention is a cell containing any resulting recombinant nucleic acid, nucleic acid libraries produced by diversity generation, recombination, or recursive recombination of the nucleic acids set forth herein, and populations of cells, vectors, viruses, plasmids or the like comprising the library or comprising any recombinant nucleic acid resulting from diversity generation or recombination (or recursive recombination) of a nucleic acid as set forth herein with another such nucleic acid, or an additional nucleic acid. Corresponding sequence strings in a database present in a computer system or computer readable medium are a feature of the invention.

### Other Polynucleotide Compositions

The invention also includes compositions comprising two or more polynucleotides of the invention (e.g., as substrates for recombination). The composition can comprise a library of recombinant nucleic acids, where the library contains at least 2, 3, 5, 10, 20, or 50 or more nucleic acids. The nucleic acids are optionally cloned into expression vectors, providing expression libraries.

The invention also includes compositions produced by digesting one or more polynucleotides of the invention with a restriction endonuclease, an RNAse, or a DNAse (e.g., as is performed in certain of the recombination formats noted above); and compositions produced by fragmenting or shearing one or more polynucleotides of the invention by mechanical means (e.g., sonication, vortexing, and the like), which can also be used to provide substrates for recombination in the methods above. Similarly, compositions comprising sets of oligonucleotides corresponding to more than one nucleic acids of the invention are useful as recombination substrates and are a feature of the invention. For convenience, these fragmented, sheared, or oligonucleotide synthesized mixtures are referred to as fragmented nucleic acid sets.

Also included in the invention are compositions produced by incubating one or more of the fragmented nucleic acid sets in the presence of ribonucleotide- or deoxyribonucelotide triphosphates and a nucleic acid polymerase. This resulting composition forms a recombination mixture for many of the recombination formats noted above. The nucleic acid polymerase may be an RNA polymerase, a DNA polymerase, or an RNA-directed DNA polymerase (e.g., a “reverse transcriptase”); the polymerase can be, e.g., a thermostable DNA polymerase (such as, VENT, TAQ, or the like).

### Interferon Homologue Polypeptides

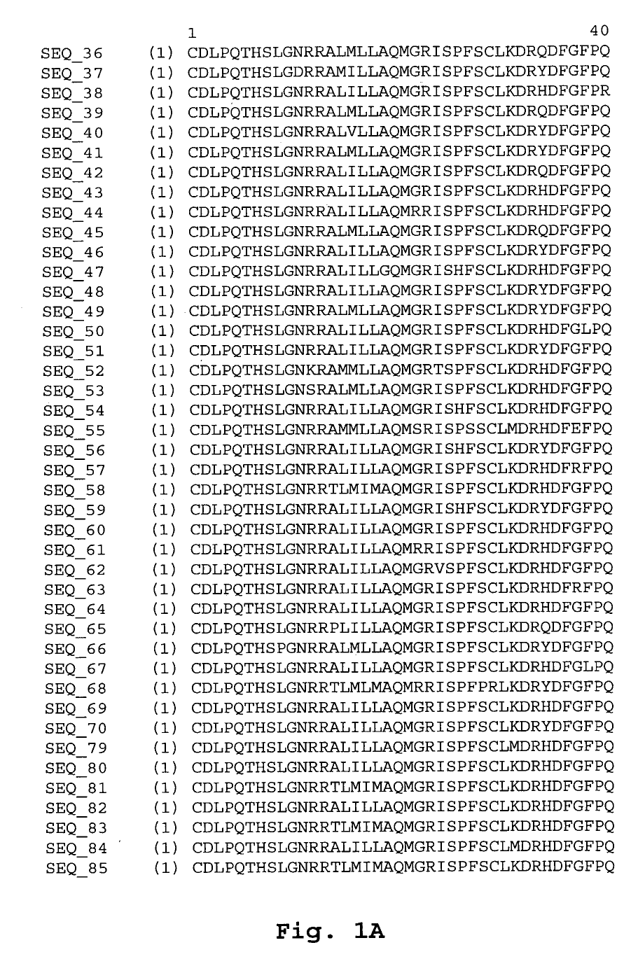

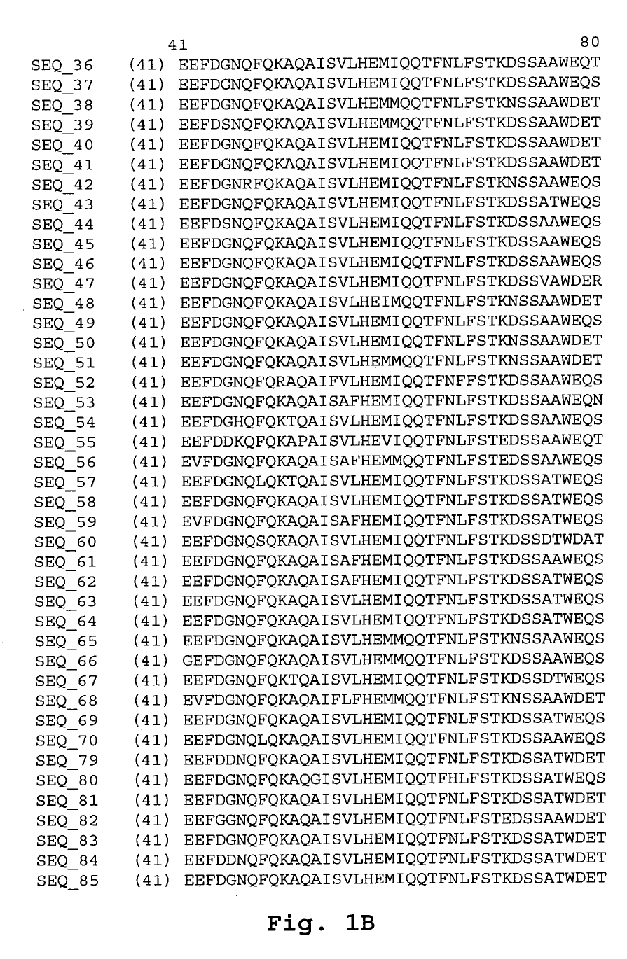

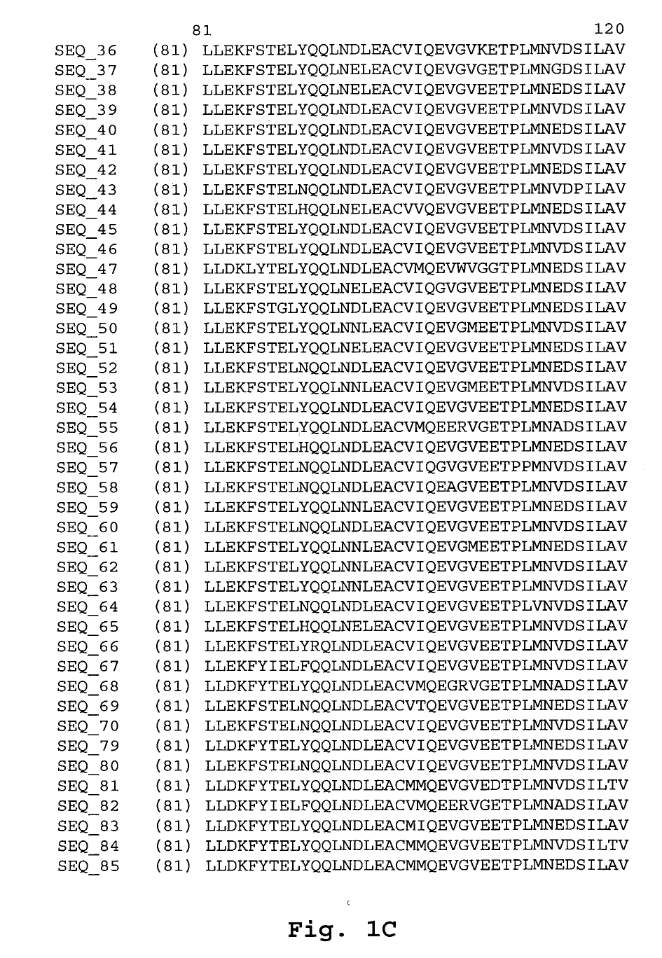

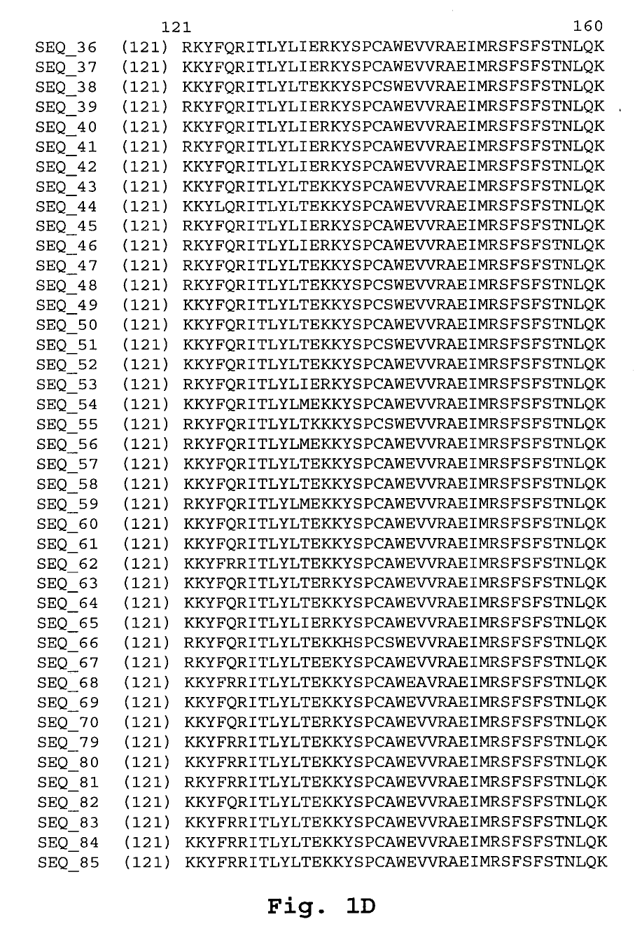

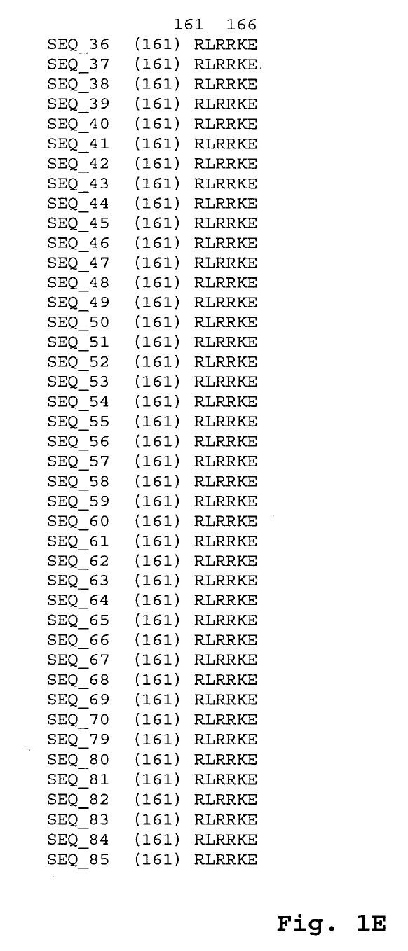

The invention provides isolated or recombinant interferon-alpha homologue polypeptides, also referred to herein as “interferon-alpha homologues,” or “interferon homologues” or “IFN-alpha homologues” or “IFN homologues”. An isolated or recombinant interferon homologue polypeptide of the invention includes a polypeptide comprising a sequence selected from SEQ ID NO:36 to SEQ ID NO:70 and SEQ ID NO:79 to SEQ ID NO:85, and conservatively modified variations thereof, and fragments thereof having an antiproliferative activity in, e.g., a human Daudi cell line-based assay (or other similar assay) and/or an antiviral activity in, e.g., a murine cell line/EMCV-based assay (or other similar assay). An alignment of exemplary interferon homologue polypeptide sequences according to the invention is provided in FIG. 1. Alignment of the polypeptide sequences of the invention to each other or to sequences of known, naturally-occurring interferon-alphas is readily performed by one of ordinary skill in the art using publicly available databases and alignment programs.

The invention also provides a polypeptide comprising at least about 100, 120, 130, 140, 150, 155, 160, 163, 165, or 166 contiguous amino acids of any one of SQ ID NOS:36-70 or SEQ ID NO:71. In one aspect, said amino acid sequence comprises amino acids Lys160 and Glu166, wherein the numbering of the amino acids in the sequence corresponds to that of SEQ ID NO:36.

Several conclusions may be drawn from comparison of the exemplary sequences of the invention (FIG. 1) to sequences of known, naturally-occurring interferon-alphas and other Type I interferons (including beta, delta, omega, and tau-interferons) from human and non-human sources. Such sequences are readily available from a variety of sources, such as GenBank, and the Pfam (Protein Families) database at http://www.sanger.ac.uk/Software/Pfam/index.shtml.

Of particular note is the presence, in some interferon homologue polypeptide sequences of the invention, of the following amino acid residues (denoted “Group I” residues) which do not appear in the equivalent position of known, naturally-occurring human or non-human Type I interferon sequences.

Group I: Asp11; Pro14; Arg50; Phe55; Asp75; Asn80; Pro111; Leu124; Glu134; Ser140, and Ala143; with residue numbering corresponding to the mature interferon homologue sequence identified as SEQ ID NO:36.

Also of note is the presence, in some interferon homologue polypeptide sequences of the invention, of the following amino acid residues (denoted “Group II” residues) which do not appear in the equivalent position of known, naturally-occurring human interferon-alpha subtype sequences.

Group II: Pro9; (Lys, Ser)12; (Thr, Val)24; Gln34; Arg40; Ser45; Arg47; Leu56; Ile60; Phe67; Ala79, Gly88; His90; Arg91; Glu95; Val101; (Gly, Ala)104; Val112; Gly114; Pro116; Lys133, and His136.

In other embodiments, the interferon homologue polypeptide comprises at least 20, 50, 100, 150, 155, or 160 of more contiguous amino acids of any one of SEQ ID NOS:36-70 and/or one or more of amino acids Ala19, (Tyr or Gln)34, Gly37, Phe38, Lys71, Ala76, Tyr90, Ile132, Arg134, Phe152, Lys160, and Glu166, wherein the numbering of the amino acids corresponds to that of SEQ ID NO:36, or one or more of amino acids Pro9, (Lys or Ser)12, (Thr or Val)24, Gln34, Arg40, Ser45, Arg47, Leu56, Ile60, Phe67, Ala79, Gly88, His90, Arg91, Glu95, Val101, (Gly, Ala)104, Val112, Gly114, Pro116, Lys133, and His136, wherein the numbering of the amino acids in said polypeptide sequence corresponds to the numbering of individual amino acids in the amino acid sequence of SEQ ID NO:36. Thus, for example, in this embodiment, an interferon polypeptide comprises an amino acid sequence comprising a proline residue at amino acid position 9 in the sequence, a lysine or serine residue at position 12, a threonine or valine residue at position 24, a glutamine residue at position 34, an arginine residue at position 40, etc. Such polypeptides may exhibit antiproliferative activities in a human Daudi cell line-based proliferation assay (e.g., at least about 8.3×10⁶ units/mg) and/or an antiviral activities in a human WISH cell/EMCV-based assay (at least about 2.1×10⁷ units/mg). Some such polypeptides bind a human alpha interferon receptor. Some such polypeptides are 166 amino acids in length. In another aspect, such polypeptides may comprise a sequence selected from any of the group of SEQ ID NO:36 to SEQ ID NO:54.

An antiproliferative activity of any polypeptide of the invention generally relates to the capability or ability of a polypeptide to cause cells or parts thereof to grow or produce new cellular growth rapidly and often repeatedly.

The invention further includes a polypeptide (e.g., any of SEQ ID NOS:36-71 or SEQ ID NOS:79-85) or a nucleic acid (e.g., any of SEQ ID NOS: 1-35 or SEQ ID NOS:72-78)encoding a polypeptide, wherein said polypeptide having an anti-angiogenic activity as measured by an anti-angiogenesis assay well known to those of ordinary skill in the art.

The invention further includes:

(a) any interferon-alpha polypeptide comprising one or more Group I amino acid residues above.

(b) any interferon-alpha polypeptide comprising one or more Group II amino acid residues above in the context of a human like interferon sequence (i.e., a sequence which displays a high level of similarity or homology to a human interferon), or a sequence which is highly similar or homologous (i.e., having a percent sequence homology or sequence identity of at least about 80%, 90%, 95%, 96%, 97%, 98% or more) to any sequence listed in the attached sequence listing or fragment thereof.

(c) any interferon-alpha polypeptide containing a combination of the following residues, which are localized in or near the regions of the interferon-alpha molecule known or proposed to interact with a Type I interferon receptor, where such sequence combinations (motifs) do not appear in the equivalent position of any known naturally-occurring human or non-human Type 1 interferon:

(i) (Tyr or Gln)34; plus one or more of Ile132 or Arg134; or

(ii) Asp78, Glu79, or (Asp or Thr)80; plus one or more of Ile132 or Arg134.

In another embodiment, the present invention provides an interferon alpha homologue comprising the sequence show in SEQ ID NO:71: CDLPQTHSLG-X₁₁-X₁₂-RA-X₁₅-X₁₆-LL-X₁₉-QM-X₂₂-R-X₂₄-S-X₂₆-FSCLKDR-X₃₄-DFG-X₃₈-P-X₄₀-EEFD-X₄₅-X₄₆-X₄₇-FQ-X₅₀-X₅₁-QAI-X₅₅-X₅₆-X₅₇-HE-X₆₀-X₆₁-QQTFN-X₆₇-FSTK-X₇₂-SS-X₇₅-X₇₆-W-X₇₈-X₇₉-X₈₀-LL-X₈₃-K-X₈₅-X₈₆-T-X₈₈-L-X₉₀-QQLN-X₉₅-LEACV-X₁₀₁-Q-X₁₀₃-V-X₁₀₅-X₁₀₆-X₁₀₇-X₁₀₈-TPLMN-X₁₁₄-D-X₁₁₆-ILAV-X₁₂₁-KY-X₁₂₄-QRITLYL-X₁₃₂-E-X₁₃₄-KYSPC-X₁₄₀-WEVVRAEIMRSFSFSTNLQKRLRRKE, or a conservatively substituted variation thereof, where X₁₁ is N or D; X₁₂ is R, S, or K; X₁₅ is L or M; X₁₆ is I, M, or V; X₁₉ is A or G; X₂₂ is G or R; X₂₄ is I or T; X₂₆ is P or H; X₃₄ is H, Y or Q; X₃₈ is F or L; X₄₀ is Q or R; X₄₅ is G or S; X₄₆ is N or H; X₄₇ is Q or R; X₅₀ is K or R; X₅₁ is A or T; X₅₅ is S or F; X₅₆ is V or A; X₅₇ is L or F; X₆₀ is M or I; X₆₁ is I or M; X₆₇ is L or F; X₇₂ is D or N; X₇₅ is A or V; X₇₆ is A or T; X₇₈ is E or D; X₇₉ is Q or E; X₈₀ is S, R, T, or N; X₈₃ is E or D; X₈₅ is F or L; X₈₆ is S or Y; X₈₈ is E or G; X₉₀ is Y, H, N; X₉₅ is D, E, or N; X₁₀₁ is I, M, or V; X₁₀₃ is E or G; X₁₀₅ is G or W; X₁₀₆ is V or M; X₁₀₇ is E, G, or K; X₁₀₈ is E or G; X₁₁₄ is V, E, or G; X₁₁₆ is S or P; X₁₂₁ is K or R; X₁₂₄ is F or L; X₁₃₂ is T, I, or M; X₁₃₄ is K or R; and X₁₄₀ is A or S; or a fragment of said SEQ ID NO:71. In another aspect, the interferon homologue polypeptide of SEQ ID NO:71, or a fragment thereof, exhibits an antiproliferative activity in a human Daudi cell line-based proliferation assay (at least about 8.3×10⁶ units/mg) and/or an antiviral activity in a human WISH cell/EMCV-based assay (at least about 2.1×10⁷ units/mg). Both such assays are discussed in greater detail below. Such polypeptide may comprise an amino acid sequence of the group of from SEQ ID NO:36 to SEQ ID NO:54 or may be encoded by a nucleotide sequence of the group of from SEQ ID NO:1 to SEQ ID NO:19.

Fragments of the interferon homologue polypeptides described herein are also a feature of the invention. An interferon alpha homologue fragment of the invention typically comprises an interferon homologue polypeptide comprising at least about 20, 25, or 30, and typically at least about 40, 50, 60, 70, 80, 90, or 100 contiguous amino acids of any one of SEQ ID NOS:36-71 or SEQ ID NOS:79-85. In other embodiments, the fragment comprises usually at least about 100, 110, 120, 125, 130, 140, 150, 155, 158, 160, 162, 163, 164, or 165 contiguous amino acids of any one of SEQ ID NOS:36-71 or SEQ ID NOS:79-85. Such polypeptide fragments may have an antiproliferative activity in a human Daudi cell line-based assay and/or an antiviral activity in a human or murine cell line/EMCV-based assay.

In other embodiments, the invention provides polypeptides having a length of 166 amino acids, and, in some such embodiments, such polypeptides have an antiproliferative activity in a human Daudi cell line-based assay (or other similar assay), including, e.g., at least about 8.3×10⁶ units/mg, and/or an antiviral activity in a human WISH cell line/EMCV-based assay (or other similar assay), including, e.g., at least about 2.1×10⁷ units/mg.

In other embodiments, the invention provides a polypeptide comprising at least 100, 150, 155, or 160 contiguous amino acids of a protein encoded by a coding polynucleotide sequence comprising any of the following: (a) SEQ ID NO:1 to SEQ ID NO:35 or SEQ ID NO:72 to SEQ ID NO:78; (b) a coding polynucleotide sequence that encodes a first polypeptide selected from any of SEQ ID NO:36 to SEQ ID NO:70 or SEQ ID NO:79 to SEQ ID NO:85; and (c) a complementary polynucleotide sequence that hybridizes under at least highly stringent (or ultra-high stringent or ultra-ultra- high stringent conditions) hybridization conditions over substantially the entire length of a polynucleotide sequence of (a) or (b). Such polypeptides may have an antiproliferative activity in a human Daudi cell line-based assay (or other similar assay), and/or an antiviral activity in a human WISH cell line/EMCV-based assay (or other similar assay). Some such polypeptides of the invention specifically bind a human alpha interferon receptor. The polypeptides and nucleic acids of the subject invention need not be identical, but can be substantially identical, to the corresponding sequence of the target molecule or related molecule, including the polypeptides of any of SEQ ID NOS:36-71 or fragments thereof (including those having antiviral or antiproliferative activities in the assays described herein), or the nucleic acids of any of SEQ ID NOS: 1-35 or fragments thereof (including those having antiviral or antiproliferative activities in the assays described herein). The polypeptides can be subject to various changes, such as insertions, deletions, and substitutions, either conservative or non-conservative, where such changes might provide for certain advantages in their use. The polypeptides of the invention can be modified in a number of ways so long as they comprise a sequence substantially identical (as defined below) or having a percent identity to a sequence in the naturally occurring or known interferon polypeptide molecule.

Alignment and comparison of relatively short amino acid sequences (less than about 30 residues) is typically straightforward. Comparison of longer sequences can require more sophisticated methods to achieve optimal alignment of two sequences. Optimal alignment of sequences for aligning a comparison window can be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Nat'l Acad. Sci. (USA) 85:2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of sequence similarity over the comparison window) generated by the various methods is selected.

The term sequence identity means that two polynucleotide sequences are identical (i.e., on a nucleotide-by-nucleotide basis) over a window of comparison. The term “percentage of sequence identity” or “percent sequence identity” is calculated by comparing two optimally aligned sequences over the window of comparison, determining the number of positions at which the identical residues occurs in both sequences to yield the number of matched positions, dividing the number of matched positions by the total number of positions in the window of comparison (i.e., the window size), and multiplying the result by 100 to yield the percentage of sequence identity. In one aspect, the present invention provides interferon homologue nucleic acids having at least about 80%, 85%, 90%, 95%, 96%, 97%, 98%, 99%, 99.5% or more percent sequence identity with the nucleic acids of any of SEQ ID NOS: 1-35 or SEQ ID NOS:72-78 or fragments thereof.

As applied to polypeptides, the term substantial identity means that two peptide sequences, when optimally aligned, such as by the programs GAP or BESTFIT using default gap weights (described in detail below), share at least about 80 percent sequence identity, preferably at least about 90 percent sequence identity, more preferably at least about 95 percent sequence identity or more (e.g., 97, 98, or 99 percent sequence identity). Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. In one aspect, the present invention provides interferon homologue polypeptides having at least about 80%, 85%, 90%, 95%, 96%, 97%, 98% 99% 99.5% or more percent sequence identity with the polypeptides of any of SEQ ID NOS:36-71 or SEQ ID NOS:79-85 or fragments thereof.

A preferred example of an algorithm that is suitable for determining percent sequence identity and sequence similarity is the FASTA algorithm, which is described in Pearson, W. R. & Lipman, D. J., 1988, Proc. Nat'l Acad. Sci. USA 85: 2444. See also W. R. Pearson, 1996, Methods Enzymol. 266: 227-258. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Another preferred example of algorithm that is suitable for determining percent sequence identity and sequence similarity are the BLAST and BLAST 2.0 algorithms, which are described in Altschul et al., 1977, Nuc. Acids Res. 25: 3389-3402 and Altschul et al., 1990, J. Mol. Biol. 215: 403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http: //www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff (1989) Proc. Nat'l Acad. Sci. U.S.A. 89: 10915) alignments (B) of 50, expectation (E) of 10, M=5, N=−4, and a comparison of both strands.

The BLAST algorithm also performs a statistical analysis of the similarity between two sequences (see, e.g., Karlin & Altschul (1993) Proc. Nat'l Acad. Sci. U.S.A. 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35: 351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al. (1984) Nuc. Acids Res. 12: 387-395.

Another preferred example of an algorithm that is suitable for multiple DNA and amino acid sequence alignments is the CLUSTALW program (Thompson, J. D. et al. (1994) Nucl. Acids. Res. 22: 4673-4680). ClustalW performs multiple pairwise comparisons between groups of sequences and assembles them into a multiple alignment based on homology. Gap open and Gap extension penalties were 10 and 0.05, respectively. For amino acid alignments, the BLOSUM algorithm can be used as a protein weight matrix (Henikoff and Henikoff (1992) Proc. Nat'l Acad. Sci. U.S.A. 89: 10915-10919).

Making Polypeptides of the Invention

Recombinant methods for producing and isolating interferon homologue polypeptides of the invention are described above. In addition to recombinant production, the polypeptides may be produced by direct peptide synthesis using solid-phase techniques (cf. Stewart et al. (1969) Solid-Phase Peptide Synthesis, W. H. Freeman Co., San Francisco; Merrifield, J. (1963) J. Am. Chem. Soc. 85:2149-2154). Peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer. For example, subsequences may be chemically synthesized separately and combined using chemical methods to provide full-length interferon homologues. Fragments of the interferon homologue polypeptides of the invention, as discussed in greater detail above, are also a feature of the invention and may be synthesized by using the procedures described above.

Polypeptides of the invention can be produced by introducing into a population of cells a nucleic acid of the invention, wherein the nucleic acid is operatively linked to a regulatory sequence effective to produce the encoded polypeptide, culturing the cells in a culture medium to produce the polypeptide, and optionally isolating the polypeptide from the cells or from the culture medium.

In another aspect, polypeptides of the invention can be produced by introducing into a population of cells a recombinant expression vector comprising at least one nucleic acid of the invention, wherein the at least one nucleic acid is operatively linked to a regulatory sequence effective to produce the encoded polypeptide, culturing the cells in a culture medium under suitable conditions to produce the polypeptide encoded by the expression vector, and optionally isolating the polypeptide from the cells or from the culture medium.

Using Polypeptides

Antibodies

In another aspect of the invention, an interferon homologue polypeptide of the invention is used to produce antibodies which have, e.g., diagnostic, prophylactic and therapeutic uses, e.g., related to the activity, distribution, and expression of interferon homologues.

Antibodies to interferon homologues of the invention may be generated by methods well known in the art. Such antibodies may include, but are not limited to, polyclonal, monoclonal, chimeric, humanized, single chain, Fab fragments and fragments produced by an Fab expression library. Antibodies, i.e., those which block receptor binding, are especially preferred for therapeutic or prophylactic use.

Interferon homologue polypeptides for antibody induction do not require biological activity; however, the polypeptide or oligopeptide must be antigenic. Peptides used to induce specific antibodies may have an amino acid sequence consisting of at least 10 amino acids, preferably at least 15 or 20 amino acids. Short stretches of an interferon homologue polypeptide may be fused with another protein, such as keyhole limpet hemocyanin, and antibody produced against the chimeric molecule.

Methods of producing polyclonal and monoclonal antibodies are known to those of skill in the art, and many antibodies are available. See, e.g., Coligan (1991) Current Protocols in Immunology Wiley/Greene, NY; and Harlow and Lane (1989) Antibodies: A Laboratory Manual, Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256:495-497. Other suitable techniques for antibody preparation include selection of libraries of recombinant antibodies in phage or similar vectors. See, Huse et al. (1989) Science 246:1275-1281; and Ward et al. (1989) Nature 341:544-546. Specific monoclonal and polyclonal antibodies and antisera will usually bind with a K_{D} of at least about 0.1 μM, preferably at least about 0.01 μM or better, and most typically and preferably, 0.001 μM or better.

Detailed methods for preparation of chimeric (humanized) antibodies can be found in U.S. Pat. No. 5,482,856. Additional details on humanization and other antibody production and engineering techniques can be found in Borrebaeck (ed.) (1995) Antibody Engineering, 2^{nd} Edition Freeman and Company, NY (Borrebaeck); McCafferty et al. (1996) Antibody Engineering, A Practical Approach, IRL at Oxford Press, Oxford, England (McCafferty), and Paul (1995) Antibody Engineering Protocols, Humana Press, Towata, N.J. (Paul).

In one useful embodiment, this invention provides for fully humanized antibodies against the interferon homologues of the invention. Humanized antibodies are especially desirable in applications where the antibodies are used as prophylactics and therapeutics in vivo and ex vivo in human patients. Human antibodies consist of characteristically human immunoglobulin sequences. The human antibodies of this invention can be produced in using a wide variety of methods (see, e.g., Larrick et al., U.S. Pat. No. 5,001,065, and Borrebaeck McCafferty and Paul, supra, for a review). In one embodiment, the human antibodies of the present invention are produced initially in trioma cells. Genes encoding the antibodies are then cloned and expressed in other cells, such as nonhuman mammalian cells. The general approach for producing human antibodies by trioma technology is described by Ostberg et al. (1983), Hybridoma 2:361-367, Ostberg, U.S. Pat. No. 4,634,664, and Engelman et al., U.S. Pat. No. 4,634,666. The antibody-producing cell lines obtained by this method are called triomas because they are descended from three cells; two human and one mouse. Triomas have been found to produce antibody more stably than ordinary hybridomas made from human cells.

Adjuvants

In one aspect, the interferon homologue polypeptides of the present invention or fragments thereof are useful as adjuvants to stimulate, enhance, potentiate, or augment an immune response related to an antigen when administered together with the antigen or after or before delivery of the antigen. In another aspect, the invention provides methods for administering one or more of the polypeptides invention described herein to a subject.

Therapeutic and Prophylactic Agents

As described in greater detail below, the interferon homologue polypeptides of the present invention or fragments thereof are useful in the prophylactic and/or therapeutic treatment of a variety of diseases, disorders, or medical conditions.

For example, the invention provides interferon-alpha homologue polypeptides (and interferon-alpha homologue nucleic acids which encode such polypeptides) that have both antiviral and antiproliferative activities in the assays described herein. In one aspect, the invention provides interferon-alpha homologue polypeptides (and interferon-alpha homologue nucleic acids which encode such polypeptides) in which the ratio of antiviral activity to antiproliferative activity is greater than that of other known interferon-alphas such as those listed in GenBank as noted herein. Such polypeptides (and nucleic acids encoding them) are useful in the therapeutic and/or prophylactic treatment of various diseases and disorders, such as, e.g., treatment regimens for hepatitis B, hepatitis C, HIV, and HSV. In such treatment regimens, some such polypeptides (and nucleic acids encoding them), such as interferon-alpha homologue 2BA8, offer significant advantages over known interferon-alpha compounds, since they likely exhibit lower side effects upon administration than known interferon-alpha compounds, such as interferon-alpha 2a, are of higher potency, and thus may require in lower dosing and cause fewer immunogenicity effects.

### Sequence Variations

Conservatively Modified Variations

Interferon homologue polypeptides of the present invention include one or more conservatively modified variations (or “conservative variations” or conservative substitutions”) of the polypeptide sequences disclosed herein as SEQ ID NO:36 to SEQ ID NO:70 and SEQ ID NO:79 to SEQ ID NO:85. Such conservatively modified variations comprise substitutions, additions or deletions which alter, add or delete a single amino acid or a small percentage of amino acids (typically less than about 5%, more typically less than about 4%, 2%, or 1%) in any of SEQ ID NO:36 to SEQ ID NO:70 and SEQ ID NO:79 to SEQ ID NO:85.

For example, a conservatively modified variation (e.g., deletion) of the 166 amino acid polypeptide identified herein as SEQ ID NO:36 has a length of at least about 157 or 158 amino acids, preferably at least about 159 or 160 amino acids, more preferably at least about 162 or 163 amino acids, and still more preferably at least about 164 or 165 amino acids, corresponding to a deletion of less than about 5%, 4%, 2% or 1% of the polypeptide sequence, respectively.

Another example of a conservatively modified variation (e.g., a “conservatively substituted variation”) of the polypeptide identified herein as SEQ ID NO:36 will contain “conservative substitutions”, according to the six substitution groups set forth in Table 2 (supra), in up to about 8 residues (i.e., less than about 5%) of the 166 amino acid polypeptide.

The interferon homologue polypeptide sequences of the invention, including conservatively substituted sequences, can be present as part of larger polypeptide sequences such as which occur upon the addition of one or more domains for purification of the protein (e.g., poly His segments, FLAG epitope segments, etc.), e.g., where the additional functional domains have little or no effect on the activity of the interferon-alpha portion of the protein, or where the additional domains can be removed by post synthesis processing steps such as by treatment with a protease.

In another embodiment, interferon homologue polypeptides of the present invention comprise the following sequence, identified herein as SEQ ID NO:71: CDLPQTHSLG-X₁₁-X₁₂-RA-X₁₅-X₁₆-LL-X₁₉-QM-X₂₂-R-X₂₄-S-X₂₆-FSCLKDR-X₃₄-DFG-X₃₈-P-X₄₀-EEFD-X₄₅-X₄₆-X₄₇-FQ-X₅₀-X₅₁-QAI-X₅₅-X₅₆-X₅₇-HE-X₆₀-X₆₁-QQTFN-X₆₇-FSTK-X₇₂-SS -X₇₅-X₇₆-W-X₇₈-X₇₉-X₈₀-LL-X₈₃-K-X₈₅-X₈₆-T-X₈₈-L-X₉₀-QQLN-X₉₅-LEACV-X₁₀₁-Q-X₁₀₃-V-X₁₀₅-X₁₀₆-X₁₀₇-X₁₀₈-TPLMN-X₁₁₄-D-X₁₁₆-ILAV-X₁₂₁-KY-X₁₂₄-QRITLYL-X₁₃₂-E-X₁₃₄-KYSPC-X₁₄₀-WEVVRAEIMRSFSFSTNLQKRLRRKE, or a conservatively substituted variation thereof, where X₁₁ is N or D; X₁₂ is R, S, or K; X₁₅ is L or M; X₁₆ is I, M, or V; X₁₉ is A or G; X₂₂ is G or R; X₂₄ is I or T; X₂₆ is P or H; X₃₄ is H, Y or Q; X₃₈ is F or L; X₄₀ is Q or R; X₄₅ is G or S; X₄₆ is N or H; X₄₇ is Q or R; X₅₀ is K or R; X₅₁ is A or T; X₅₅ is S or F; X₅₆ is V or A; X₅₇ is L or F; X₆₀ is M or I; X₆₁ is I or M; X₆₇ is L or F; X₇₂ is D or N; X₇₅ is A or V; X₇₆ is A or T; X₇₈ is E or D; X₇₉ is Q or E; X₈₀ is S, R, T, or N; X₈₃ is E or D; X₈₅ is F or L; X₈₆ is S or Y; X₈₈ is E or G; X₉₀ is Y, H, N; X₉₅ is D, E, or N; X₁₀₁ is I, M, or V; X₁₀₃ is E or G; X₁₀₅ is G or W; X₁₀₆ is V or M; X₁₀₇ is E, G, or K; X₁₀₈ is E or G; X₁₁₄ is V, E, or G; X₁₁₆ is S or P; X₁₂₁ is K or R; X₁₂₄ is F or L; X₁₃₂ is T, I, or M; X₁₃₄ is K or R; and X₁₄₀ is A or S; or a fragment of said SEQ ID NO:71. As defined above, a conservatively modified variation of the sequence of SEQ ID NO:71 can include up to a total of about 8 amino acid deletions, insertions, or conservative substitutions in the 166 amino acid polypeptide, excluding the positions designated X in SEQ ID NO:71, which correspond to the amino acid explicitly defined.

As an example, if four conservative substitutions were localized in the subsequence corresponding to amino acids 141-166 of SEQ ID NO:71, examples of conservatively substituted variations of this subsequence,

WEVVR AEIMR SFSFS TNLQK RLRRKE, include:

WEVVR SEIMR SFSYS TNLQR RLRRKD and

WELVR AEIVR SFSFS TNLNK RLRKKE, and the like, where the conservative substitutions are underlined.

A feature of the invention is an interferon homologue polypeptide comprising at least about 20, usually at least about 25, typically at least about 30, 40, 50, 60, 70, 80, 90, or 100 contiguous amino acids of any one of SEQ ID NOS:36-71 or SEQ ID NOS:79-85. In other embodiments, the polypeptide typically comprises at least about 100, 110, 120, 125, 130, 140, 150, 155, 158, 160, 163, 164, or 165 contiguous amino acids of any one of SEQ ID NOS:36-70 or SEQ ID NOS:79-85.

In other embodiments, the interferon homologue polypeptide of the invention comprises an amino acid sequence comprising one or more of amino acid residues (Tyr or Gln)34, Gly37, Phe38, Lys71, Ala76, Tyr90, Ile132, Arg134, Phe152, Lys160, and Glu166, wherein the numbering of the amino acids corresponds to the numbering of amino acids in the amino acid sequence of SEQ ID NO:36. In a preferred embodiment, the interferon homologue polypeptide comprises an amino acid sequence comprising at least 150, 155, or 166 contiguous amino acid residues of any one of SEQ ID NOS:36-70, further comprising Lys160 and Glu166, wherein the numbering of the amino acids corresponds to the numbering of amino acids in the amino acid sequence of SEQ ID NO:36. Some such polypeptides also exhibit an antiproliferative activity of at least about 8.3×10⁶ units/milligram in the human Daudi cell line-based assay, or an antiviral activity of at about least 2.1×10⁷ units/milligram (mg) in the human WISH cell/EMCV-based assay.

### Defining Polypeptides by Immunoreactivity

Because the polypeptides of the invention provide a variety of new polypeptide sequences as compared to other alpha interferon homologues, the polypeptides also provide a new structural features which can be recognized, e.g., in immunological assays. The generation of antisera which specifically binds the polypeptides of the invention, as well as the polypeptides which are bound by such antisera, are features of the invention.

The invention includes interferon-alpha homologue polypeptides that specifically bind to or that are specifically immunoreactive with an antibody or antisera generated against an immunogen comprising an amino acid sequence selected from one or more of SEQ ID NO:36 to SEQ ID NO:70, SEQ ID NO:71, and SEQ ID NO:79 to SEQ ID NO:85. To eliminate cross-reactivity with other interferon-alpha polypeptides, e.g., known interferon-alpha polypeptides, the antibody or antisera (or antiserum) is subtracted with available known alpha interferons, such as those polypeptides encoded by nucleic acids represented by GenBank accession numbers J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), and M38289, V00549 (alpha-2a), and I08313 (alpha-Con1), or any other known interferon-alpha polypeptides (typically referred to as the “control alpha interferon polypeptides”). Where the accession number corresponds to a nucleic acid, a polypeptide encoded by the nucleic acid is generated and used for antibody/antisera subtraction purposes. Where the nucleic acid corresponds to a non-coding sequence, e.g., a pseudo gene, an amino acid which corresponds to the reading frame of the nucleic acid is generated (e.g., synthetically), or is minimally modified to include a start codon for recombinant production.

In one typical format, the immunoassay uses a polyclonal antiserum which was raised against one or more polypeptides comprising one or more of the amino acid sequences corresponding to one or more of: SEQ ID NO:36 to SEQ ID NO:70, SEQ ID NO:71, and SEQ ID NO:79 to SEQ ID NO:85, or a substantial subsequence thereof (i.e., at least about 30%, 40%, 50%, 60%, 70%, 80%, 90%, 95%, or 98% or more of the full length sequence provided). The full set of potential polypeptide immunogens derived from one or more of SEQ ID NO:36 to SEQ ID NO:70, SEQ ID NO:7, and SEQ ID NO:79 to SEQ ID NO:85 are collectively referred to below as “the immunogenic polypeptides.” The resulting antisera is optionally selected to have low cross-reactivity against the control alpha interferon polypeptides and/or other known interferon polypeptides and any such cross-reactivity is removed by immunoabsorption with one or more of the control alpha interferon polypeptides, prior to use of the polyclonal antiserum in the immunoassay.

In order to produce antisera for use in an immunoassay, one or more of the immunogenic polypeptides is produced and purified as described herein. For example, recombinant protein may be produced in a mammalian cell line. An inbred strain of mice (used in this assay because results are more reproducible due to the virtual genetic identity of the mice) is immunized with the immunogenic polypeptide(s) in combination with a standard adjuvant, such as Freund's adjuvant, and a standard mouse immunization protocol (see Harlow and Lane (1988) Antibodies, A Laboratory Manual, Cold Spring Harbor Publications, New York, for a standard description of antibody generation, immunoassay formats and conditions that can be used to determine specific immunoreactivity). Alternatively, one or more synthetic or recombinant polypeptides derived from the sequences disclosed herein is conjugated to a carrier protein and used as an immunogen.

Polyclonal sera are collected and titered against the immunogenic polypeptide(s) in an immunoassay, for example, a solid phase immunoassay with one or more of the immunogenic polypeptides immobilized on a solid support. Polyclonal antisera with a titer of 10⁶ or greater are selected, pooled and subtracted with the control alpha interferon polypeptides to produce subtracted pooled titered polyclonal antisera.

The subtracted pooled titered polyclonal antisera are tested for cross reactivity against the control alpha interferon polypeptides. Preferably at least two of the immunogenic alpha interferon polypeptides are used in this determination, preferably in conjunction with at least two of the control alpha interferon polypeptides, to identify antibodies which are specifically bound by the immunogenic polypeptides(s).

In this comparative assay, discriminatory binding conditions are determined for the subtracted titered polyclonal antisera which result in at least about a 5-10 fold higher signal to noise ratio for binding of the titered polyclonal antisera to the immunogenic alpha interferons as compared to binding to the control alpha interferons. That is, the stringency of the binding reaction is adjusted by the addition of non-specific competitors such as albumin or non-fat dry milk, or by adjusting salt conditions, temperature, or the like. These binding conditions are used in subsequent assays for determining whether a test polypeptide is specifically bound by the pooled subtracted polyclonal antisera. In particular, test polypeptides which show at least a 2-5× higher signal to noise ratio than the control polypeptides under discriminatory binding conditions, and at least about a ½ signal to noise ratio as compared to the immunogenic polypeptide(s), shares substantial structural similarity or homology with the immunogenic polypeptide as compared to known alpha interferons, and is, therefore a polypeptide of the invention.

In another example, immunoassays in the competitive binding format are used for detection of a test polypeptide. For example, as noted, cross-reacting antibodies are removed from the pooled antisera mixture by immunoabsorption with the control alpha interferon polypeptides. The immunogenic polypeptide(s) are then immobilized to a solid support which is exposed to the subtracted pooled antisera. Test proteins are added to the assay to compete for binding to the pooled subtracted antisera. The ability of the test protein(s) to compete for binding to the pooled subtracted antisera as compared to the immobilized protein(s) is compared to the ability of the immunogenic polypeptide(s) added to the assay to compete for binding (the immunogenic polypeptides compete effectively with the immobilized immunogenic polypeptides for binding to the pooled antisera). The percent cross-reactivity for the test proteins is calculated, using standard calculations.

In a parallel assay, the ability of the control proteins to compete for binding to the pooled subtracted antisera is determined as compared to the ability of the immunogenic polypeptide(s) to compete for binding to the antisera. Again, the percent cross-reactivity for the control polypeptides is calculated, using standard calculations. Where the percent cross-reactivity is at least 5-10× as high for the test polypeptides, the test polypeptides are said to specifically bind the pooled subtracted antisera.

In general, the immunoabsorbed and pooled antisera can be used in a competitive binding immunoassay as described herein to compare any test polypeptide to the immunogenic polypeptide(s). In order to make this comparison, the two polypeptides are each assayed at a wide range of concentrations and the amount of each polypeptide required to inhibit 50% of the binding of the subtracted antisera to the immobilized protein is determined using standard techniques. If the amount of the test polypeptide required is less than twice the amount of the immunogenic polypeptide that is required, then the test polypeptide is said to specifically bind to an antibody generated to the immunogenic polypeptide, provided the amount is at least about 5-10× as high as for a control polypeptide.

As a final determination of specificity, the pooled antisera is optionally fully immunosorbed with the immunogenic polypeptide(s) (rather than the control polypeptides) until little or no binding of the resulting immunogenic polypeptide subtracted pooled antisera to the immunogenic polypeptide(s) used in the immunoabsorption is detectable. This fully immunosorbed antisera is then tested for reactivity with the test polypeptide. If little or no reactivity is observed (i.e., no more than 2× the signal to noise ratio observed for binding of the fully immunosorbed antisera to the immunogenic polypeptide), then the test polypeptide is specifically bound by the antisera elicited by the immunogenic protein.

### Antiproliferative Properties of Interferon Homologues

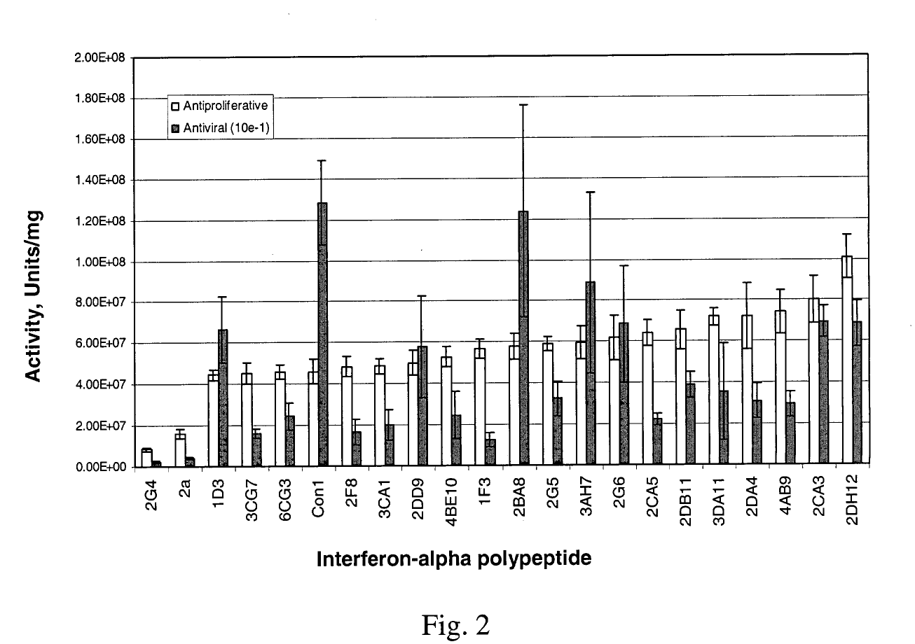

The effect of interferon homologues on cellular growth was examined in a human Daudi cell line-based assay as described in Example 1. FIG. 2 shows the antiproliferative activity of exemplary interferon homologues of the invention comprising amino acid sequences SEQ ID NO:36 to SEQ ID NO:54, in comparison to control interferons, human IFN-alpha 2a and consensus human IFN-alpha (Con1). The graph shows the number of Units of activity per milligram (mg) of interferon test sample (Y axis) for a set of exemplary interferon alpha homologues, each of which is designated with a name (clone name) on the X axis, compared with that of human IFN-alpha 2a and consensus human IFN-alpha. These results indicate that compositions comprising an interferon-alpha homologue of the present invention can be used in methods to inhibit or reduce proliferation of tumor cells, including, but not limited to: human carcinoma cells, hematopoietic cancer cells, human leukemia cells, human lymphoma cells, and human melanoma cells. Inhibition can be performed in vitro (useful, e.g., in a variety of proliferation assays), ex vivo or in vivo (useful, e.g., as a therapeutic or prophylactic agent).

Interferon-alpha homologues of the present invention show diverse activity patterns against a variety of cancer cell lines (see, e.g., Example 2). An in vitro cell line screen (as described in, e.g., Monks, A. et al. (1991) J. Nat'l Cancer Inst. 83:757-766) was used to assay interferon-alpha homologues of the invention for selective growth inhibition and/or cell killing of particular cancer cell lines. The human cancer cell lines screened (see, e.g., Example 2, Table 3) include leukemias, melanomas, and cancers of the lung, colon, brain, central nervous system, ovary, breast, prostate, and kidney.

Three activity parameters were determined in the cancer cell line screen: 1) GI50 (“growth inhibition at 50%”), a measure of growth inhibition activity, is the concentration of interferon test sample (IFN alpha homologue or control IFN alpha) at which cell growth is inhibited by 50%, as measured by a 50% reduction in the net protein/polypeptide increase in the interferon test sample as compared to that observed in the control cells (no test sample) at the end of the incubation period; 2) TGI (“total growth inhibition”) a measure of cytostatic activity, is the concentration of interferon test sample at which cell growth of a particular cell line is totally inhibited, wherein the amount of cellular protein at the end of the incubation period equals the amount of cellular protein at the beginning of the incubation period; and 3) LC50, a measure of cytotoxic activity, is the concentration of interferon test sample at which a 50% reduction in the measured amount of cellular protein at the end of the incubation as compared to that at the beginning of the incubation period is observed, indicating a net loss of cells following interferon test sample addition. Further details of the assay and data analysis procedures are provided in Example 2.

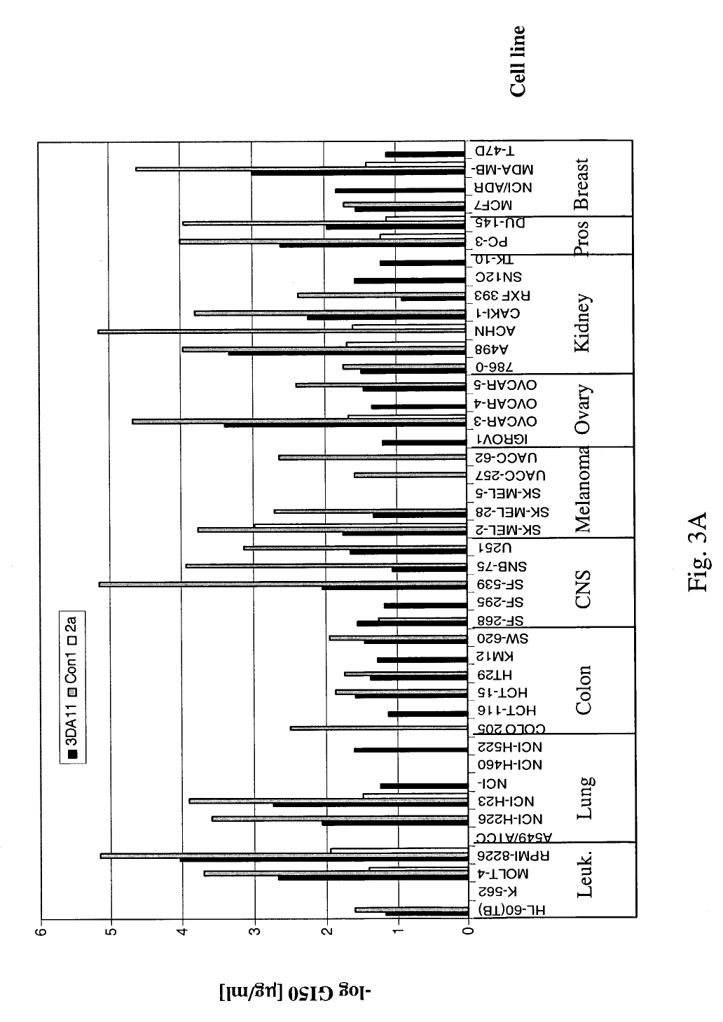

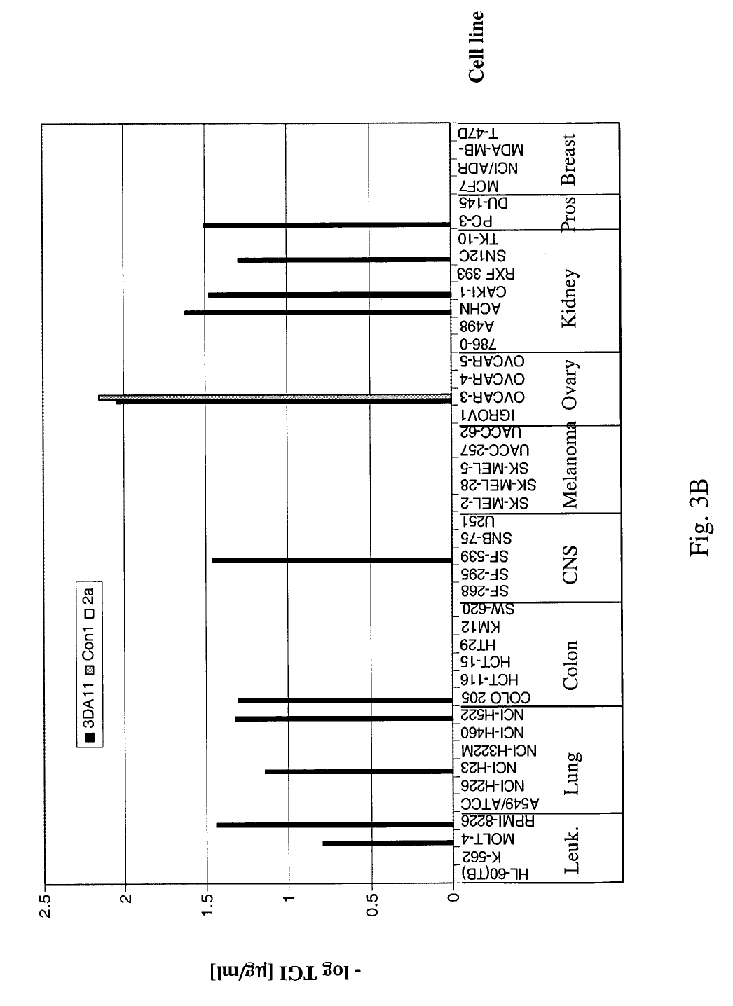

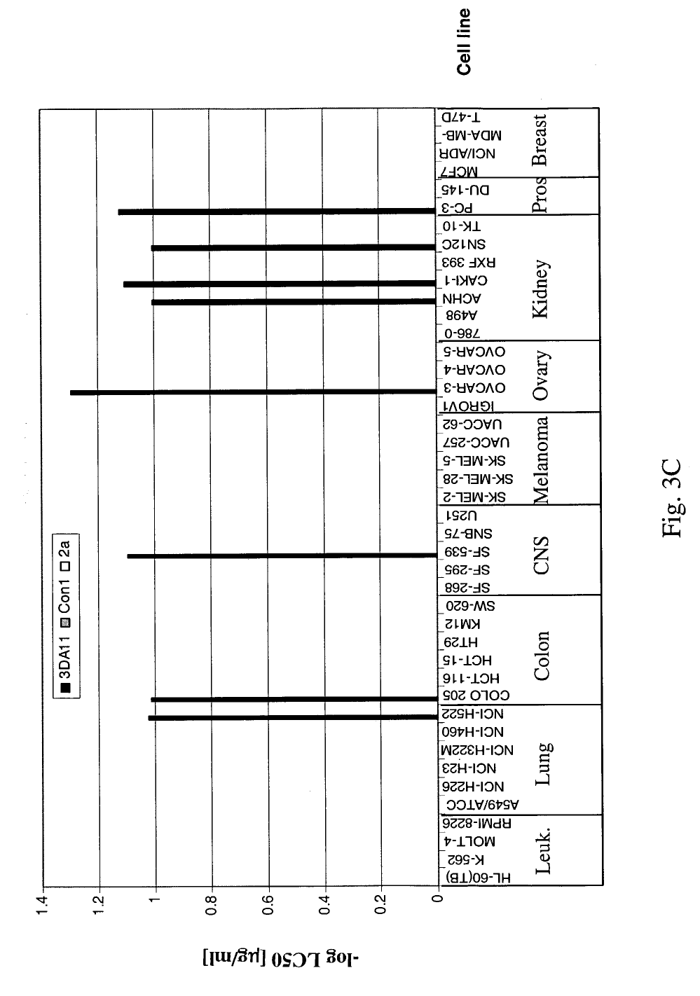

The activity parameters of exemplary interferon-alpha homologue 3DA11 (SEQ ID NO:40) against a variety of cancer cell lines are shown in FIGS. 3A, 3B, and 3C, in comparison with the interferon-alpha Con1 and human interferon-alpha 2a controls.

With respect to growth inhibition activity, in particular, homologue 3DA11 and control interferon-alpha Con1 showed significant activity against most of the cell lines tested, with the interferon-alpha Con1 exhibiting generally higher activity, and interferon-alpha 2a generally exhibiting lower overall activity and in only a subset of the cell lines (FIG. 3A).

In contrast, in particular, a pronounced difference was observed in the cytotoxic and cytostatic activities of homologue 3DA11 in comparison to both interferon-Con1 and human interferon-alpha 2a controls. In the concentration range tested, homologue 3DA 11 showed significant cytostatic activity against a population of cells of eleven of the cell lines, while interferon-Con1 showed activity against only a population of cells of one of the cell lines, against which homologue 3DA11 was also active (FIG. 3B). IFN-alpha 2a, on the other hand, was not active in this assay against any of the tested cell lines. Homologue 3DA11 thus has a broader cytostatic activity profile than consensus human interferon-alpha (Con1) and human interferon-alpha 2a.

Homologue 3DA11 also showed significant cytotoxic activity in comparison to the interferon-Con1 and human interferon-alpha 2a controls (FIG. 3C). Surprisingly, homologue 3DA11 displayed cytotoxic activity against a population of cells of 8 of the cell lines, whereas neither the interferon-Con1 nor the interferon-alpha 2a controls exhibited measurable activity against a population of cells of any of the cell lines at the concentration range employed in the assay. Thus, homologue 3DA11 also has a broader cytotoxic activity profile than interferon-Con1 and human interferon-alpha 2a.

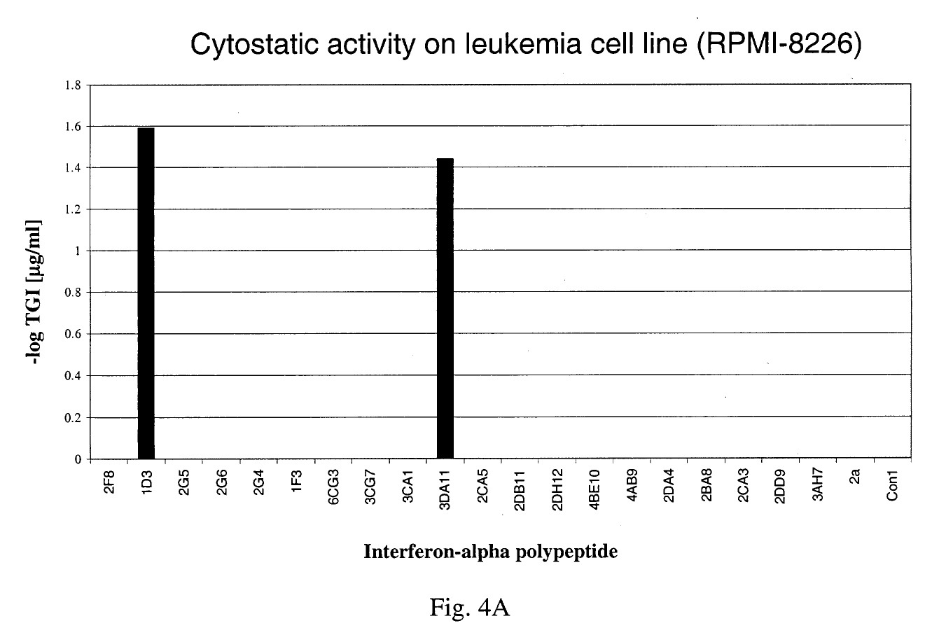

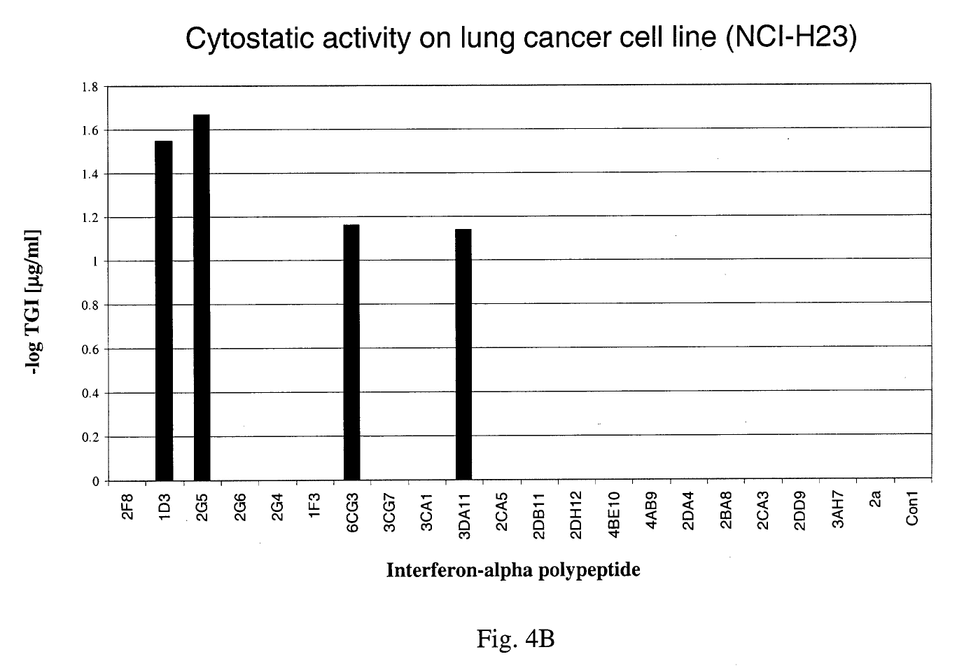

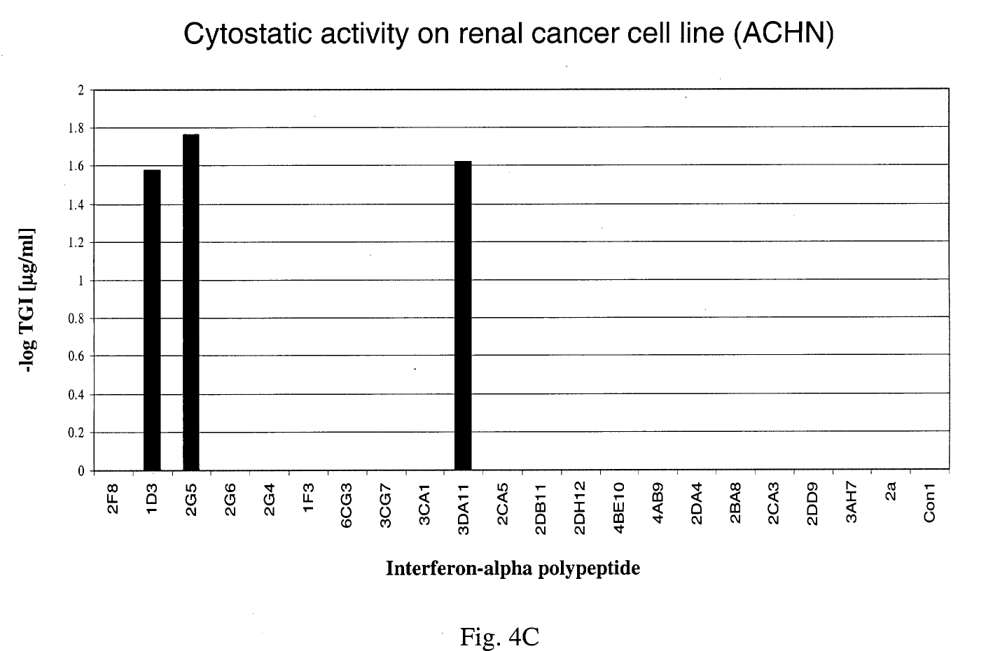

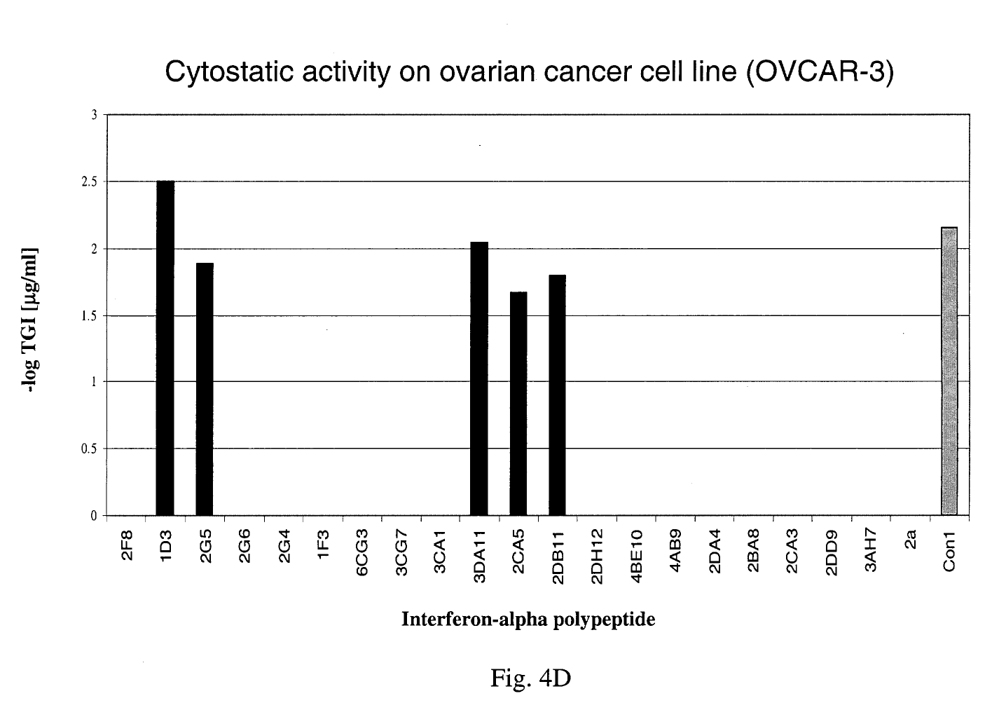

FIGS. 4A-4D illustrate the cytostatic activity (as reflected by the TGI value) of exemplary interferon-alpha homologues of the invention. In each figure, the relative cytostatic activity (expressed as -log TGI) against a population of cells of particular cancer cell line is plotted for various interferon-alpha homologues and for the two control interferons (interferon-Con1 and human interferon-alpha 2a).

Of the exemplary homologues tested, homologues 1D3 (SEQ ID NO:54) and 3DA11 (SEQ ID NO:40), but neither of the control interferons, exhibited significant cytostatic activity against a population of cells of leukemia cell line RPMI-8226 over the concentration range of the assay (FIG. 4A). In this example, the 1D3 and 3DA11 homologues showed at least about 25-fold higher cytostatic activity against a population of the cells (corresponding to a difference in TGI of at least about 1.4 log units) than did either of the controls (interferon-Con1 or interferon-alpha 2a) against a population of cells of the leukemia cell line.

Homologues 1D3, 2G5 (SEQ ID NO:45), 6CG3 (SEQ ID NO:52) and 3DA11, but neither of the control interferons, exhibited significant cytostatic activity against lung cancer cell line NCI-H23 (FIG. 4B). In this example, the 1D3, 2G5, 6CG3, and 3DA11 homologues showed at least about 12-fold higher cytostatic activity a population of cells of a lung cancer cell line (corresponding to a difference in TGI of at least about 1.1 log units) than either interferon-Con1 or interferon-alpha 2a against a population of cells of the lung cancer cell line.

Homologues 1D3, 2G5, and 3DA 11, but neither of the control interferons, showed significant cytostatic activity against a population of cells of renal cancer cell line ACHN (FIG. 4C). In this example, the 1D3, 2G5, and 3DA11 homologues showed at least about 35-fold higher cytostatic activity a population of cells of said renal cancer cell line (corresponding to a difference in TGI of at least about 1.55 log units) than either interferon-Con1 or interferon-alpha 2a against a population of cells of renal cancer cell line.

Homologues 1D3, 2G5, 3DA11, 2CA5 (SEQ ID NO:42) and 2DB11 (SEQ ID NO:41), and the interferon-Con1 control, but not the interferon alpha-2a control, exhibited significant cytostatic activity against a population of cells of an ovarian cancer cell line OVCAR-3 (FIG. 4D). In this example, homologue 1D3 showed at least about 2-fold higher cytostatic activity (corresponding to a difference in TGI of at least about 0.3 log units) than interferon-Con1, and the 1D3, 2G5, 3DA11, 2CA5, and 2DB 11 homologues showed at least about 40-fold higher cytostatic activity (corresponding to a difference in TGI of at least about 1.6 log units) than interferon-alpha 2a, against respective populations of cells of the ovarian cancer cell line.

From the exemplary data provided herein, it is apparent that interferon-alpha homologues of the invention showed a variety of cytostatic activity profiles, which differed significantly from those of the interferon-alpha Con1 and interferon alpha-2a.

The present invention includes an interferon-alpha homologue having increased cytostatic activity relative to human interferon-alpha 2a or to consensus human interferon-alpha, Con1. In various embodiments, the interferon-alpha homologue has at least about 2-fold higher cytostatic activity a population of cells of a cancer cell line (i.e., has a TGI value at least about 2-fold lower) than does human interferon-alpha 2a, or has at least 2-fold higher cytostatic activity than interferon-Con1, against a population of cells of one or more cancer cell lines selected from the following: a leukemia cell line; a melanoma cell line; a lung cancer cell line; a colon cancer cell line; a central nervous system (CNS) cancer cell line; an ovarian cancer cell line; a breast cancer cell line; a prostate cancer cell line; and a renal cancer cell line.

In other embodiments, the interferon-alpha homologue has at least about 5-fold higher cytostatic activity a population of cells of a cancer cell line (i.e., has a TGI value at least about 5-fold lower) than does human interferon-alpha 2a, or has at least about 5-fold higher cytostatic activity than interferon-Con1, against a population of cells of one or more cancer cell lines selected from the following: a leukemia cell line; a melanoma cell line; a lung cancer cell line; a colon cancer cell line; a central nervous system (CNS) cancer cell line; an ovarian cancer cell line; a breast cancer cell line; a prostate cancer cell line; and a renal cancer cell line. In other embodiments, the interferon-alpha homologue has at least about 10-fold higher cytostatic activity a population of cells of a cancer cell line (i.e., has a TGI value at least about 10-fold lower) than does human interferon-alpha 2a, or has at least about 10-fold higher cytostatic activity than interferon-Con1, against a population of cells of one or more cancer cell lines selected from the following: a leukemia cell line; a melanoma cell line; a lung cancer cell line; a colon cancer cell line; a CNS cancer cell line; an ovarian cancer cell line; a breast cancer cell line; a prostate cancer cell line; and a renal cancer cell line.

The invention includes an interferon-alpha homologue having increased cytotoxic activity relative to human interferon-alpha 2a or relative to interferon-Con1. In various embodiments, the interferon-alpha homologue has at least about 2-fold higher cytotoxic activity (i.e., has an LC50 value at least about 2-fold lower), at least 5-fold higher cytotoxic activity, or at least 10-fold higher cytotoxic activity, than human interferon-alpha 2a against a population of cells of one or more cancer cell lines selected from the following: a leukemia cell line; a melanoma cell line; a lung cancer cell line; a colon cancer cell line; a CNS cancer cell line; an ovarian cancer cell line; a breast cancer cell line; a prostate cancer cell line; and a renal cancer cell line. In other embodiments, the interferon-alpha homologue has at least about 2-fold higher cytotoxic activity (i.e., has an LC50 value at least about 2-fold lower), at least about 5-fold higher cytotoxic activity, or at least about 10-fold higher cytotoxic activity, than interferon-Con1, against a population of cells of at least one cancer cell line selected from: a leukemia cell line; a melanoma cell line; a lung cancer cell line; a colon cancer cell line; a CNS cancer cell line; an ovarian cancer cell line; a breast cancer cell line; a prostate cancer cell line; and a renal cancer cell line.

The invention includes an interferon-alpha homologue having increased growth inhibition activity relative to human interferon-alpha 2a or to interferon-Con1. In various embodiments, the interferon-alpha homologue has at least about 2-fold higher growth inhibition activity (i.e., has a GI50 value at least about 2-fold lower), at least about 5-fold higher growth inhibition activity, or at least about 10-fold higher growth inhibition activity, than human interferon-alpha 2a, against a population of cells of one or more cancer cell lines selected from: a leukemia cell line; a melanoma cell line; a lung cancer cell line; a colon cancer cell line; a CNS cancer cell line; an ovarian cancer cell line; a breast cancer cell line; a prostate cancer cell line; and a renal cancer cell line. In other embodiments, the interferon-alpha homologue has at least about 2-fold higher growth inhibition activity (i.e., has a GI50 value at least about 2-fold lower), at least about 5-fold higher growth inhibition activity, or at least about 10-fold higher growth inhibition activity, than interferon-Con1, against at least one cancer cell line selected from the following: a leukemia cell line; a melanoma cell line; a lung cancer cell line; a colon cancer cell line; a CNS cancer cell line; an ovarian cancer cell line; a breast cancer cell line; a prostate cancer cell line; and a renal cancer cell line.

The discovery set forth herein that interferons (such as the interferon-alpha homologues described herein) can be evolved, modified, or recombined to display a variety of activity profiles provides an opportunity for evolving and creating customized and specific interferon homologues for the treatment of a variety of specific diseases or disease conditions, including, e.g., a variety of cancers or related conditions. For example, an interferon homologue of the invention optimized to have increased potency against a particular target cancer cell type may also be optimized to have (advantageously) reduced toxicity towards a non-target cell(s), and thus may produce lower side effects in the subject to which the homologue is administered (e.g., patient).

The present invention further provides an opportunity to optimize interferon homologues against tumor cells taken from a subpopulation of subjects (e.g., mammals or human patients), or even from an individual subject (e.g., mammal or human patient), providing therapeutic or prophylactic treatment tailored to the individual subject. Optimized interferon homologues of the invention may provide therapeutic or prophylactic benefit against cancers or related conditions or other interferon-treatable disorders or conditions which are otherwise unresponsive to currently-available interferons or to other treatment regimes.

### Antiviral Properties of Interferon Homologues

The antiviral activity of interferon homologues of the present invention was evaluated in a human WISH cell EMCV assay as described in Example 1. FIG. 2 shows the antiviral activity of exemplary interferon homologues of the invention comprising amino acid sequences SEQ ID NO:36 to SEQ ID NO:54.

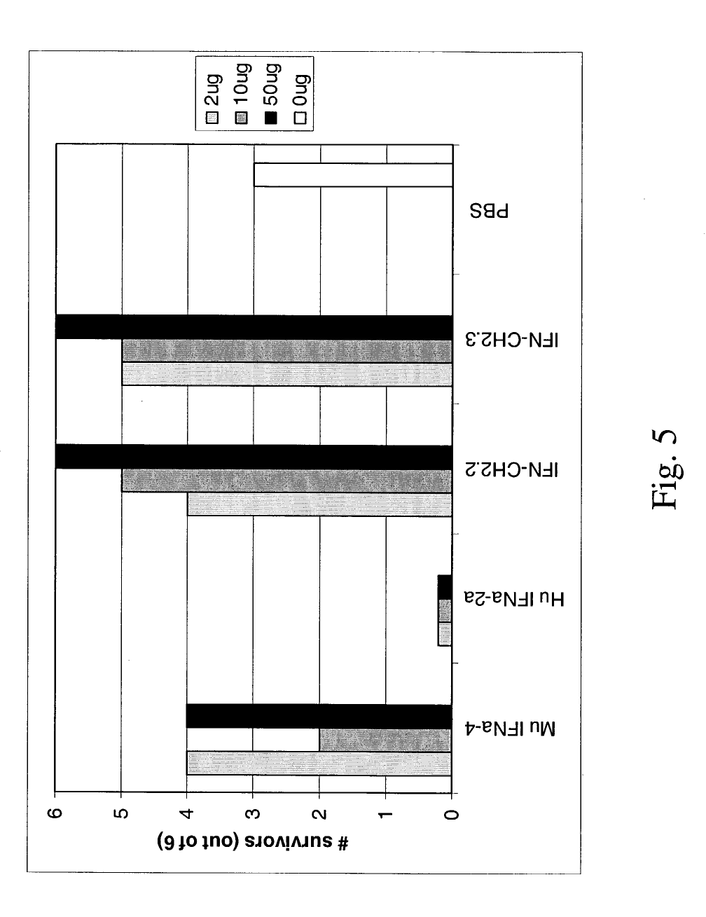

Improved in vitro antiviral activity of exemplary IFN-alpha homologues of the invention has been shown to be maintained in vivo in a murine model system. Two IFN-alpha homologues of the invention, designated CH2.2 and CH2.3 (SEQ ID NOS:84 and 85, respectively), were previously shown to have about 206,000-fold and 138,000-fold improved antiviral activity, respectively, compared to human IFN-alpha 2a in a murine cell-based assay, as well as significantly higher activity in the same assay as compared to native murine interferons (Chang et al. (1999) Nature Biotechnol. 17:793-797). As described in Example 3 below, Balb/c mice challenged with a lethal dose of vesicular stomatitis virus (VSV) were administered varying doses of IFN-alpha homologues, designated CH2.2 and CH2.3, native murine interferon Mu-IFN alpha 4, and human IFN-alpha 2a. The high in vitro activity correlated well with the observed in vivo activity (FIG. 5). The CH2.2 and CH2.3 homologues were fully effective in protecting mice from the lethal viral challenge, while the same dosage of the native murine interferon was partially effective and the human IFN-alpha 2a was completely ineffective. These results indicate that compositions comprising interferon homologues of the present invention can be used in methods to inhibit viral replication in subjects infected with viruses including, but not limited to: human immunodeficiency virus (HIV), hepatitis C virus (HCV), herpes simplex virus (HSV), and hepatitis B virus (HBV). Inhibition can be performed in vitro (useful, e.g., in a variety of antiviral assays), ex vivo (useful e.g., as a therapeutic or prophylactic agent in ex vivo methods discussed herein), or in vivo ( useful, e.g., as a therapeutic or prophylactic agent in in vivo methods discussed herein).

### Interferon Homologues in the Treatment of Autoimmune and Other Immune-related Disorders

Compositions of the present invention can be used to therapeutically or prophylactically treat and thereby alleviate a variety of immune system-related disorders characterized by hyper- or hypo-active immune system function or other features. Such disorders include hyperallergenicity and autoimmune disorders, such as multiple sclerosis, type I (insulin dependent) diabetes mellitus, lupus erythematosus, amyotrophic lateral sclerosis, Crohn's disease, rheumatoid arthritis, stomatitis, asthma, allergies, psoriasis and the like.

### Therapeutic and Prophylactic Compositions

Therapeutic or prophylactic compositions comprising one or more interferon homologue polypeptides or nucleic acids of the invention are tested in appropriate in vitro, ex vivo, and in vivo animal models of disease, to confirm efficacy, tissue metabolism, and to estimate dosages, according to methods well known in the art. In particular, dosages can be determined by activity comparison of the alpha interferon homologues to existing alpha interferon therapeutics or prophylactics, i.e., in a relevant assay. In one aspect, the invention provides methods comprising administering one or more interferon homologue nucleotides or polypeptides of the invention (or fragments thereof) described above to a mammal, including, e.g., a human, primate, mouse, pig, cow, goat, rabbit, rat, guinea pig, hamster, horse, sheep; or a non-mammalian vertebrate such as a bird (e.g., a chicken or duck) or a fish, or invertebrate, as described in greater detail below. Such compositions typically comprise one or more interferon homologue nucleotides or polypeptides of the invention (or fragments thereof) and an excipient, including, e.g., a pharmaceutically acceptable excipient.

In one aspect, a composition of the invention is produced by digesting one or more nucleic acids of the invention (or fragments thereof) with a restriction endonuclease, an RNase, or a DNase.

In another aspect of the invention, compositions produced by incubating one or more nucleic acids described above in the presence of deoxyribonucelotide triphosphates and a nucleic acid polymerase, e.g., a thermostable polymerase, are provided.

The invention also includes compositions comprising two or more nucleic acids described above. The composition may comprise a library of nucleic acids, where the library contains at least about 5, 10, 20, 50, 100, 150, or 200 or more such nucleic acids.

Administration is by any of the routes normally used for introducing a molecule into ultimate contact with blood or tissue cells. The interferon-alpha homologues of the invention are administered in any suitable manner, preferably with pharmaceutically acceptable carriers. Suitable methods of administering such interferon homologues in the context of the present invention to a patient are available, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention.

Polypeptide compositions can be administered for any of the prophylactic, therapeutic, and diagnostic methods described herein by a number of routes including, but not limited to oral, intravenous, intraperitoneal, intramuscular, transdermal, subcutaneous, topical, sublingual, vaginal, or rectal means, or by inhalation. Interferon homologue polypeptide compositions can also be administered via liposomes. Such administration routes and appropriate formulations are generally known to those of skill in the art.

The interferon homologue polypeptide or nucleic acid, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of packaged nucleic acid can be presented in unit-dose or multi-dose sealed containers, such as ampules and vials.

Parenteral administration and intravenous administration are preferred methods of administration. In particular, the routes of administration already in use for existing alpha interferon therapeutics or prophylactics, along with formulations in current use, are preferred routes of administration and formulation for the alpha interferon homologue polypeptide and nucleic acids of the invention.

Cells transduced with the interferon homologue nucleic acids as described above in the context of ex vivo or in vivo therapy can also be administered intravenously or parenterally as described above. It will be appreciated that the delivery of cells to subjects (e.g., human patients) is routine, e.g., delivery of cells to the blood via intravenous or intraperitoneal administration.

The dose of interferon homologue polypeptide or nucleic acid of the invention administered to a subject (e.g., patient), in the context of the present invention is sufficient to effect a beneficial therapeutic or prophylactic response in the subject (e.g., patient) over time, or to inhibit infection by a pathogen, depending on the application. The dose will be determined by the efficacy of the particular vector, or formulation, and the activity interferon homologue employed and the condition of the patient, as well as the body weight or surface area of the patient to be treated. The size of the dose also will be determined by the existence, nature, and extent of any adverse side-effects that accompany the administration of a particular vector, formulation, transduced cell type or the like in a particular patient.

In the therapeutic and prophylactic treatment methods of the invention described herein, an effective amount of an interferon-alpha nucleic acid (e.g., DNA or mRNA) of the invention (e.g., nucleic acid dosage) will generally be in the range of, e.g., from about 0.05 microgram/kilogram (kg) to about 50 mg/kg, usually about 0.005-5 mg/kg. However, as will be understood, the effective amount of the nucleic acid (e.g., nucleic acid dosage) and/or polpeptide (e.g., polypeptide dosage) will vary in a manner apparent to those of ordinary skill in the art according to a number of factors, including the activity or potency of the polypeptide, the activity or potency of any nucleic acid construct (e.g., vector, promoter, expression system) to be administered, the disease or condition (e.g., particular cancer) to be treated, and the subject to which or whom the nucleic acid is delivered.

For delivery of some polypeptides, e.g., by delivering nucleic acids encoding such polypeptides, for example, adequate levels of translation and/or expression are achieved with a nucleic acid dosage of, e.g., about 0.005 mg/kg to about 5 mg/kg. Dosages for other polypeptides (and nucleic acids encoding them) having a known biological activity can be readily determined by those of skill in the art according to the factors noted above. Dosages used for other known interferon-alphas for particular diseases provide guidelines for determining dosage and treatment regimen for a nucleic acid or polypeptide of the invention. An effective amount of an interferon-alpha homologue polypeptide may be in the range of from about 1 microgram to about 1 milligram, and more typically from about 1 microgram to about 100 micrograms.

A composition for use in therapeutic and prophylactic treatment methods of the invention described herein may comprise, e.g., a concentration of an interferon-alpha homologue nucleic acid (e.g., DNA or mRNA) of the invention of from about 0.1 microgram/milliliter (ml) to about 20 mg/ml and a pharmaceutically acceptable carrier (e.g., aqueous carrier).

A composition for use in therapeutic and prophylactic treatment methods of the invention described herein may comprise, e.g., a concentration of an interferon-alpha homologue polypeptide of the invention in an amount as described above and herein and a pharmaceutically acceptable carrier (e.g., aqueous carrier).

In determining the effective amount of the vector, cell type, or formulation to be administered in the treatment or prophylaxis of cancers or viral diseases, the physician evaluates circulating plasma levels, vector/cell/formulation/interferon homologue toxicities, progression of the disease, and the production of anti-vector/interferon homologue antibodies.

The dose administered, e.g., to a 70 kilogram patient will be in the range equivalent to dosages of currently-used interferon-alpha therapeutic or prophylactic proteins, and doses of vectors or cells which produce interferon homologue sequences are calculated to yield an equivalent amount of interferon homologue nucleic acid or expressed protein. The vectors of this invention can supplement treatment of cancers and virally-mediated conditions by any known conventional therapy, including cytotoxic agents, nucleotide analogues (e.g., when used for treatment of HIV infection), biologic response modifiers, and the like.

For administration, interferon homologues and transduced cells of the present invention can be administered at a rate determined by the LD-50 of the interferon homologue polypeptide or nucleic acid, vector, or transduced cell type, and the side-effects of the interferon homologue polypeptides or nucleic acids, vector or cell type at various concentrations, as applied to the mass and overall health of the patient. Administration can be accomplished via single or divided doses.

For introduction of recombinant alpha-interferon nucleic acid transduced cells into a subject (e.g., patient), blood samples are obtained prior to infusion, and saved for analysis. Between 1×10⁶ and 1×10¹² transduced cells are infused intravenously over 60-200 minutes. Vital signs and oxygen saturation by pulse oximetry are closely monitored. Blood samples are obtained 5 minutes and 1 hour following infusion and saved for subsequent analysis. Leukopheresis, transduction and reinfusion are optionally repeated every 2 to 3 months for a total of 4 to 6 treatments in a one year period. After the first treatment, infusions can be performed on a outpatient basis at the discretion of the clinician. If the reinfusion is given as an outpatient, the participant is monitored for at least 4, and preferably 8 hours following the therapy. Transduced cells are prepared for reinfusion according to established methods. See Abrahamsen et al. (1991) J. Clin. Apheresis 6:48-53; Carter et al. (1988) J. Clin. Arpheresis 4:113-117; Aebersold et al. (1988), J. Immunol. Methods 112:1-7; Muul et al. (1987) J. Immunol. Methods 101:171-181 and Carter et al. (1987) Transfusion 27:362-365. After a period of about 2-4 weeks in culture, the cells should number between 1×10⁶ and 1×10¹². In this regard, the growth characteristics of cells vary from patient to patient and from cell type to cell type. About 72 hours prior to reinfusion of the transduced cells, an aliquot is taken for analysis of phenotype, and percentage of cells expressing the therapeutic or prophylactic agent.

If a subject (e.g., patient) undergoing infusion of a vector or transduced cell or protein formulation develops fevers, chills, or muscle aches, he/she receives the appropriate dose of aspirin, ibuprofen, acetaminophen or other pain/fever controlling drug. Subjects (e.g., patients) who experience reactions to the infusion such as fever, muscle aches, and chills are premedicated 30 minutes prior to the future infusions with either aspirin, acetaminophen, or, e.g., diphenhydramine. Meperidine is used for more severe chills and muscle aches that do not quickly respond to antipyretics and antihistamines. Cell infusion is slowed or discontinued depending upon the severity of the reaction.

### Therapeutic and Prophylactic Treatment Methods

The present invention also includes methods of therapeutically or prophylactically treating a disease or disorder by administering in vivo or ex vivo one or more nucleic acids or polypeptides of the invention described above (or compositions comprising a pharmaceutically acceptable excipient and one or more such nucleic acids or polypeptides) to a subject, including, e.g., a mammal, including, e.g., a human, primate, mouse, pig, cow, goat, rabbit, rat, guinea pig, hamster, horse, sheep; or a non-mammalian vertebrate such as a bird (e.g., a chicken or duck) or a fish, or invertebrate.

In one aspect of the invention, in ex vivo methods, one or more cells or a population of cells of interest of the subject (e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc.) are obtained or removed from the subject and contacted with an amount of a polypeptide of the invention that is effective in prophylactically or therapeutically treating the disease, disorder, or other condition. The contacted cells are then returned or delivered to the subject to the site from which they were obtained or to another site (e.g., including those defined above) of interest in the subject to be treated. If desired, the contacted cells may be grafted onto a tissue, organ, or system site (including all described above) of interest in the subject using standard and well-known grafting techniques or, e.g., delivered to the blood or lymph system using standard delivery or transfusion techniques.

The invention also provides in vivo methods in which one or more cells or a population of cells of interest of the subject are contacted directly or indirectly with an amount of a polypeptide of the invention effective in prophylactically or therapeutically treating the disease, disorder, or other condition. In direct contact/administration formats, the polypeptide is typically administered or transferred directly to the cells to be treated or to the tissue site of interest (e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc.) by any of a variety of formats, including topical administration, injection (e.g., by using a needle or syringe), or vaccine or gene gun delivery, pushing into a tissue, organ, or skin site. The polypeptide can be delivered, for example, intramuscularly, intradermally, subdermally, subcutaneously, orally, intraperitoneally, intrathecally, intravenously, or placed within a cavity of the body (including, e.g., during surgery), or by inhalation or vaginal or rectal administration.

In in vivo indirect contact/administration formats, the polypeptide is typically administered or transferred indirectly to the cells to be treated or to the tissue site of interest, including those described above (such as, e.g., skin cells, organ systems, lymphatic system, or blood cell system, etc.), by contacting or administering the polypeptide of the invention directly to one or more cells or population of cells from which treatment can be facilitated. For example, tumor cells within the body of the subject can be treated by contacting cells of the blood or lymphatic system, skin, or an organ with a sufficient amount of the polypeptide such that delivery of the polypeptide to the site of interest (e.g., tissue, organ, or cells of interest or blood or lymphatic system within the body) occurs and effective prophylactic or therapeutic treatment results. Such contact, administration, or transfer is typically made by using one or more of the routes or modes of administration described above.

In another aspect, the invention provides ex vivo methods in which one or more cells of interest or a population of cells of interest of the subject (e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc.) are obtained or removed from the subject and transformed by contacting said one or more cells or population of cells with a polynucleotide construct comprising a target nucleic acid sequence of the invention that encodes a biologically active polypeptide of interest (e.g., a polypeptide of the invention) that is effective in prophylactically or therapeutically treating the disease, disorder, or other condition. The one or more cells or population of cells is contacted with a sufficient amount of the polynucleotide construct and a promoter controlling expression of said nucleic acid sequence such that uptake of the polynucleotide construct (and promoter) into the cell(s) occurs and sufficient expression of the target nucleic acid sequence of the invention results to produce an amount of the biologically active polypeptide effective to prophylactically or therapeutically treat the disease, disorder, or condition. The polynucleotide construct may include a promoter sequence (e.g., CMV promoter sequence) that controls expression of the nucleic acid sequence of the invention and/or, if desired, one or more additional nucleotide sequences encoding at least one or more of another polypeptide of the invention, a cytokine, adjuvant, or co-stimulatory molecule, or other polypeptide of interest.

Following transfection, the transformed cells are returned, delivered, or transferred to the subject to the tissue site or system from which they were obtained or to another site (e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc.) to be treated in the subject. If desired, the cells may be grafted onto a tissue, skin, organ, or body system of interest in the subject using standard and well-known grafting techniques or delivered to the blood or lymphatic system using standard delivery or transfusion techniques. Such delivery, administration, or transfer of transformed cells is typically made by using one or more of the routes or modes of administration described above. Expression of the target nucleic acid occurs naturally or can be induced (as described in greater detail below) and an amount of the encoded polypeptide is expressed sufficient and effective to treat the disease or condition at the site or tissue system.

In another aspect, the invention provides in vivo methods in which one or more cells of interest or a population of cells of the subject (e.g., including those cells and cells systems and subjects described above) are transformed in the body of the subject by contacting the cell(s) or population of cells with (or administering or transferring to the cell(s) or population of cells using one or more of the routes or modes of administration described above) a polynucleotide construct comprising a nucleic acid sequence of the invention that encodes a biologically active polypeptide of interest (e.g., a polypeptide of the invention) that is effective in prophylactically or therapeutically treating the disease, disorder, or other condition.

The polynucleotide construct can be directly administered or transferred to cell(s) suffering from the disease or disorder (e.g., by direct contact using one or more of the routes or modes of administration described above). Alternatively, the polynucleotide construct can be indirectly administered or transferred to cell(s) suffering from the disease or disorder by first directly contacting non-diseased cell(s) or other diseased cells using one or more of the routes or modes of administration described above with a sufficient amount of the polynucleotide construct comprising the nucleic acid sequence encoding the biologically active polypeptide, and a promoter controlling expression of the nucleic acid sequence, such that uptake of the polynucleotide construct (and promoter) into the cell(s) occurs and sufficient expression of the nucleic acid sequence of the invention results to produce an amount of the biologically active polypeptide effective to prophylactically or therapeutically treat the disease or disorder, and whereby the polynucleotide construct or the resulting expressed polypeptide is transferred naturally or automatically from the initial delivery site, system, tissue or organ of the subject's body to the diseased site, tissue, organ or system of the subject's body (e.g., via the blood or lymphatic system). Expression of the target nucleic acid occurs naturally or can be induced (as described in greater detail below) such that an amount of the encoded polypeptide is expressed sufficient and effective to treat the disease or condition at the site or tissue system. The polynucleotide construct may include a promoter sequence (e.g., CMV promoter sequence) that controls expression of the nucleic acid sequence and/or, if desired, one or more additional nucleotide sequences encoding at least one or more of another polypeptide of the invention, a cytokine, adjuvant, or co-stimulatory molecule, or other polypeptide of interest.

In each of the in vivo and ex vivo treatment methods as described above, a composition comprising an excipient and the polypeptide or nucleic acid of the invention can be administered or delivered. In one aspect, a composition comprising a pharmaceutically acceptable excipient and a polypeptide or nucleic acid of the invention is administered or delivered to the subject as described above in an amount effective to treat the disease or disorder.

In another aspect, in each in vivo and ex vivo treatment method described above, the amount of polynucleotide administered to the cell(s) or subject can be an amount sufficient that uptake of said polynucleotide into one or more cells of the subject occurs and sufficient expression of said nucleic acid sequence results to produce an amount of a biologically active polypeptide effective to enhance an immune response in the subject, including an immune response induced by an immunogen (e.g., antigen). In another aspect, for each such method, the amount of polypeptide administered to cell(s) or subject can be an amount sufficient to enhance an immune response in the subject, including that induced by an immunogen (e.g., antigen).

In yet another aspect, in an in vivo or in vivo treatment method in which a polynucleotide construct (or composition comprising a polynucleotide construct) is used to deliver a physiologically active polypeptide to a subject, the expression of the polynucleotide construct can be induced by using an inducible on- and off-gene expression system. Examples of such on- and off-gene expression systems include the Tet-On™ Gene Expression System and Tet-Off™ Gene Expression System (see, e.g., Clontech Catalog 2000, pg. 110-111 for a detailed description of each such system), respectively. Other controllable or inducible on- and off-gene expression systems are known to those of ordinary skill in the art. With such system, expression of the target nucleic of the polynucleotide construct can be regulated in a precise, reversible, and quantitative manner. Gene expression of the target nucleic acid can be induced, for example, after the stable transfected cells containing the polynucleotide construct comprising the target nucleic acid are delivered or transferred to or made to contact the tissue site, organ or system of interest. Such systems are of particular benefit in treatment methods and formats in which it is advantageous to delay or precisely control expression of the target nucleic acid (e.g., to allow time for completion of surgery and/or healing following surgery; to allow time for the polynucleotide construct comprising the target nucleic acid to reach the site, cells, system, or tissue to be treated; to allow time for the graft containing cells transformed with the construct to become incorporated into the tissue or organ onto or into which it has been spliced or attached, etc.)

### Integrated Systems

The present invention provides computers, computer readable media and integrated systems comprising character strings corresponding to the sequence information herein for the polypeptides and nucleic acids herein, including, e.g., those sequences listed herein and the various silent substitutions and conservative substitutions thereof.

Various methods and genetic algorithms (GOs) known in the art can be used to detect homology or similarity between different character strings, or can be used to perform other desirable functions such as to control output files, provide the basis for making presentations of information including the sequences and the like. Examples include BLAST, discussed supra.

Thus, different types of homology and similarity of various stringency and length can be detected and recognized in the integrated systems herein. For example, many homology determination methods have been designed for comparative analysis of sequences of biopolymers, for spell-checking in word processing, and for data retrieval from various databases. With an understanding of double-helix pair-wise complement interactions among 4 principal nucleobases in natural polynucleotides, models that simulate annealing of complementary homologous polynucleotide strings can also be used as a foundation of sequence alignment or other operations typically performed on the character strings corresponding to the sequences herein (e.g., word-processing manipulations, construction of figures comprising sequence or subsequence character strings, output tables, etc.). An example of a software package with GOs for calculating sequence similarity or homology is BLAST, which can be adapted to the present invention by inputting character strings corresponding to the sequences herein.

Similarly, standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention by inputting a character string corresponding to the interferon alpha homologues of the invention (either nucleic acids or proteins, or both). For example, the integrated systems can include the foregoing software having the appropriate character string information, e.g., used in conjunction with a user interface (e.g., a GUI in a standard operating system such as a Windows, Macintosh or LINUX system) to manipulate strings of characters. As noted, specialized alignment programs such as BLAST can also be incorporated into the systems of the invention for alignment of nucleic acids or proteins (or corresponding character strings).

Integrated systems for analysis in the present invention typically include a digital computer with GO software for aligning sequences, as well as data sets entered into the software system comprising any of the sequences herein. The computer can be, e.g., a PC (Intel x86 or Pentium chip-compatible DOS™, OS2™ WINDOWS™ WINDOWS NT™, WINDOWS95™, WINDOWS98™ LINUX based machine, a MACINTOSH™, Power PC, or a UNIX based (e.g., SUN™ work station) machine) or other commercially common computer which is known to one of skill. Software for aligning or otherwise manipulating sequences is available, or can easily be constructed by one of skill using a standard programming language such as Visualbasic, Fortran, Basic, Java, or the like.

Any controller or computer optionally includes a monitor which is often a cathode ray tube (“CRT”) display, a flat panel display (e.g., active matrix liquid crystal display, liquid crystal display), or others. Computer circuitry is often placed in a box which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of sequences to be compared or otherwise manipulated in the relevant computer system.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation.

The software can also include output elements for controlling nucleic acid synthesis (e.g., based upon a sequence or an alignment of a sequences herein) or other operations which occur downstream from an alignment or other operation performed using a character string corresponding to a sequence herein.

In one embodiment, the invention provides an integrated system comprising a computer or computer readable medium comprising a database having one or more sequence records. Each of the sequence records comprises one or more character strings corresponding to a nucleic acid or polypeptide or protein sequence selected from SEQ ID NO:1 to SEQ ID NO:85. The integrated system further comprises a use input interface allowing a use to selectively view the one or more sequence records. In one such integrated system, the computer or computer readable medium comprises an alignment instruction set that aligns the character strings with one or more additional character strings corresponding to a nucleic acid or polypeptide or protein sequence.

One such integrated system includes an instruction set that comprises at least one of the following: a local homology comparison determination, a homology alignment determination, a search for similarity determination, and a BLAST determination. In some embodiments, the system further comprises a readable output element that displays an alignment produced by the alignment instruction set. In another embodiment, the computer or computer readable medium further comprises an instruction set that translates at least one nucleic acid sequence which comprises a sequence selected from SEQ ID NO:1 to SEQ ID NO:35 or SEQ ID NO:72 to SEQ ID NO:78 into an amino acid sequence. The instruction set may select the nucleic acid by applying a codon usage instruction set or an instruction set which determines sequence identity to a test nucleic acid sequence.

Methods of using a computer system to present information pertaining to at least one of a plurality of sequence records stored in a database are also provided. Each of the sequence records comprises at least one character string corresponding to SEQ ID NO:1 to SEQ ID NO:85. The method comprises determining at least one character string corresponding to one or more of SEQ ID NO:1 to SEQ ID NO:85 or a subsequence thereof; determining which of the at least one character string of the list are selected by a user; and displaying each of the selected character strings, or aligning each of the selected character strings with an additional character string. The method may further comprise displaying an alignment of each of the selected character strings with an additional character string and/or displaying the list.

### Kits

In an additional aspect, the present invention provides kits embodying the methods, composition, systems and apparatus herein. Kits of the invention optionally comprise one or more of the following: (1) an apparatus, system, system component or apparatus component as described herein; (2) instructions for practicing the methods described herein, and/or for operating the apparatus or apparatus components herein and/or for using the compositions herein; (3) one or more alpha interferon homologue compositions (such as e.g., compositions comprising at least one interferon alpha homologue nucleic acid or polypeptide or fragment thereof, cell, vector, etc., of the invention) or components (interferon alpha homologue nucleic acid or polypeptide or fragment thereof, cell, vector, etc., of the invention); (4) a container for holding one or more aspects of the invention, including such components or compositions, and (5) packaging materials.

In a further aspect, the present invention provides for the use of any apparatus, apparatus component, composition or kit herein, for the practice of any method or assay herein, and/or for the use of any apparatus or kit to practice any assay or method herein.

### EXAMPLES

### Example I: Preparation and Screening of Shuffled Interferon-alpha Libraries

Fragments (25-60 base pairs (bp) in length) of about 20 human interferon-alpha subspecies genes were prepared by PCR amplification and DNAse treatment, and recombined essentially as described in Crameri A. et al. (1998; Nature 15:288-291), to produce shuffled interferon-alpha mature coding sequences. Expression libraries were prepared by subcloning shuffled interferon-alpha mature coding sequences into an E. coli secretion vector. Shuffled interferon polypeptides were expressed as mature proteins fused at the C-termini to an E tag (Amersham-Pharmacia) to facilitate quantitation and purification from the periplasmic space. E. coli transformants were picked using a robotic colony picker (Q-Bot, Genetix Pharmaceuticals) into microtiter plates, and periplasmic extracts were prepared.

Periplasmic extracts were assayed for antiproliferative activity on a human Daudi cell line as described by Scarozza, A. M. et al. (1992) J. Interferon Res. 12:35-42.

Clones exhibiting antiproliferative activity in the Daudi assay were re-screened and expression levels determined by Western blot using an anti-E tag antibody (Amersham-Pharmacia). Clones exhibiting highest activity normalized to expression levels were selected for sequencing and were also utilized as substrates for additional rounds of shuffling and screening as described above.

Clones from the first and second rounds of shuffling having relatively high antiproliferative activity by the Daudi assay were subcloned into a CHO expression vector (pDEI-1011) in which the E-tag/6-His tag (Amersham-Pharmacia) is fused to the C-terminus of the shuffled interferons. Clones were transfected into CHO cells and stable cell lines were selected with 1 mg/ml G418. CHO-expressed mature interferons were purified on anti-E tag Sepharose column (Amersham-Pharmacia) and quantitated by a Bradford assay (Biorad). CHO-purified shuffled interferons were assayed for antiproliferative activity by the Daudi assay and for antiviral activity using a human WISH cell/EMCV assay as described below.

Human WISH Cell/EMCV Antiviral Assay

WISH cells were seeded to a density of 6×10⁴ cells/well in 96-well plates in 100 ul RPMI medium (Gibco-BRL) supplemented with 10% fetal calf serum, penicillin (100 μg/ml), and streptomycin (100 μg/ml), and incubated for 24 hours at 37° C. Samples of interferon-alpha polypeptides in medium (100 μl total volume) were added to wells and incubated for 3 hours at 37° C. under a 5% CO₂ atmosphere. Dilutions of EMCV (encephalomyocarditis virus) were added to wells in 50 μl volumes, and incubated for 24 hours as above. Medium was carefully removed and wells were rinsed 2× with warm phosphate-buffered saline (PBS). Neutral red (100 μl/well of 1:50 dilution in medium) was added to the wells and incubated for 2 hours as above. Glutaraldehyde (50 μl/well of 0.5% in PBS) was added and incubated for 30 minutes as above. Wells were washed 2× in PBS, and 100 μl/well of a solution of 50% methanol, 1% acetic acid was added. Absorbance at 540 nanometers (nm) was measured using a microplate reader.

FIG. 2 shows the antiproliferative activity and the antiviral activity of exemplary interferon homologues of the invention, in comparison with interferon alpha-2a and interferon-alpha Con1. The graph shows the number of Units activity per milligram of homologue (Y axis) for a set of exemplary interferon alpha homologues, each of which is designated with a “name” on the X axis.

### Example 2: In vitro Cancer Cell Line Screen

An in vitro cell line screen (as described in, e.g., Monks, A. et al. (1991) J. Nat'l Cancer Inst. 83:757-766 (hereinafter “Monks”) and http://dtp.nci.gov./branches/btb/ivclsp.html, each of which is incorporated herein by reference in its entirety for all purposes) was used to assay interferon-alpha homologues of the invention for selective growth inhibition and/or cell killing of particular cancer cell lines. The 60 human cancer cell lines used (Table 3) include leukemias, melanomas, and cancers of the lung, colon, brain, ovary, breast, prostate, central nervous system, renal system, and kidney. Human tumor cell lines were grown according to procedures outlined in Monks”) and http:/dtp.nci.gov./branches/btb/ivclsp.html.

TABLE 3
Human cancer cell lines screened

| Cancer type | Cell lines |
|---|---|
| Leukemia | CCRF-CEM, HL-60 (TB), K-562, MOLT-4, |
| | RPMI-8226, SR |
| Colon cancer | COLO 205, HCC-2998, HCT-15, HCT-116, |
| | HT29, KM12, SW-620 |
| CNS cancer | SF-268, SF-295, SF-539, SNB-19, SNB-75, U251 |
| Lung cancer | A549/ATCC, EKVX, HOP-62, HOP-92, NCI-H23, |
| | NCI-H226, NCI-H322M, NCI-H460, NCI-H522 |
| Breast cancer | MCF-7, NCI/ADR HS578T, MDA-MB-231/ATCC, |
| | MDA-MB-435, MDA-N, BT-549, T-47D |
| Melanoma | LOX IMVI, M14, MALME-3M, SK-MEL-2, |
| | SK-MEL-5, SK-MEL-28, UACC-62, UACC-257 |
| Ovarian cancer | IGROV1, OVCAR-3, OVCAR-4, OVCAR-5, |
| | OVCAR-8, SK-OV-3 |
| Prostate cancer | DU-145, PC-3 |
| Renal cancer | 786-0, A498, ACHIN, CAKI-1, RXF 393, SN12C, |
| | TK-10, UO-31 |

Briefly, cells were inoculated into 96 well microtiter plates at densities ranging from about 5,000 to about 40,000 cells/well, depending on the growth properties of the particular cell line. After inoculation, the microtiter plates were incubated for 24 hours (h) at 37 degrees C. prior to addition of test samples (e.g., interferon homologues of the invention or control interferons). After 24 h, two plates of each cell line were fixed in situ with trichloroacetic acid (TCA), to provide a measurement of the cell population for each cell line at the time of test sample addition (T₀). To the remaining plates, interferon samples (affinity-purified from CHO cell supernatants) were added in five 10-fold serial dilutions ranging from 10^{-0.8} to 10^{-4.8} μg/ml.

Following sample addition, the plates were incubated for an additional 6 days. The assay was terminated by addition of TCA.

Cell population was determined by measuring cellular protein in a quantitative protein dye-binding assay. Sulforhodamine B solution (100 μl) at 0.4 % (w/v) in 1% acetic acid was added to each well, followed by incubation for 10 minutes at room temperature. Unbound dye was removed by washing five times with 1% acetic acid and the plates air-dried. Protein-bound dye was solubilized with 10 milliMolar (mM) Tris, and the absorbance read at 515 nanometer (nm) on an automated plate reader.

Seven absorbance measurements were taken for each dose-response assay, corresponding to: the amount of cellular protein prior to sample addition (time zero; T₀), the amount of cellular protein at the end of the incubation period in the absence of test sample (control growth, C), and five measurements corresponding to the amount of cellular protein at the end of the incubation period in the presence of each of the five concentrations of interferon test sample (test growth in presence of interferon test sample at the five concentration levels, Tᵢ). These measurements were used to calculate the following three parameters for each test sample:

GI50, or “growth inhibition of 50%,” is the concentration of interferon test sample at which cell growth is inhibited by 50%, as measured by a 50% reduction in the net protein/polypeptide increase in the interferon test sample as compared to that observed in the control cells (no test sample) at the end of the incubation period. GI50 is calculated as the concentration of test sample where [(Tᵢ−T₀)/(C−T₀)]×100=50. See FIG. 3A.

TGI, or “total growth inhibition,” is the concentration of interferon test sample at which cell growth is totally inhibited, wherein the amount of cellular protein at the end of the incubation period equals the amount of cellular protein at the beginning of the incubation period. The concentration of interferon test sample that produces total growth inhibition (TGI) is calculated as the concentration of test sample where Tᵢ=T₀.

LC50 is the concentration of interferon test sample at which a 50% reduction in the measured amount of cellular protein at the end of the incubation as compared to that at the beginning of the incubation period is observed, indicating a net loss of cells following interferon test sample addition. LC50 is calculated as the concentration of test sample where [(Tᵢ−T₀)/T₀]×100=−50.

If, for a particular test sample, an effect was not achieved or was exceeded at the concentration range tested, the value for that parameter was expressed as greater or less than the maximum or minimum concentration tested.

### Example 3: In vitro Activity of IFN-alpha Homologues Correlates with in vivo Efficacy

Fragments of human interferon-alpha genes were shuffled and screened for activity in a murine cell-based antiviral assay as described by Chang et al. (1999) Nature Biotechnol. 17:793-797. Interferon-alpha homologues that exhibited over 10⁵-fold higher antiviral activity than human interferon-alpha 2a against mouse cells were isolated. The antiviral activities of a number of the interferon-alpha homologues even significantly exceeded the antiviral activity of native mouse interferons, including Mu-IFN-alpha 4 (Chang et al., supra). Recursive sequence recombination (e.g., DNA shuffling) of human interferon-alpha gene fragments to produce novel interferon alpha homologues and subsequent screening of such homologues against murine interferon receptors resulted in the identification and isolation of interferon-alpha homologues with activity optimized for the distantly related murine species.

A dose-response study in mice was performed to determine if the high antiviral activity observed in vitro is sustained in vivo. Two of the mouse-optimized interferon-alpha homologues, designated herein as CH2.2 and CH2.3 (SEQ ID NOS:84 and 85, respectively), were used in this study. CH2.2 and CH2.3 were shown to have about 138,000-fold and about 206,00-fold higher activity, respectively, than human interferon-alpha 2a, and about 2.5-fold and about 1.6-fold higher activity than native mouse interferon-alpha 4, in the in vitro mouse cell antiviral assay (Chang et al., supra).

Groups of Balb/c mice received subcutaneous doses of either phosphate buffered saline (PBS), interferon-alpha homologue CH2.2, interferon-alpha homologue CH2.3, murine IFN-alpha 4, or human interferon-alpha 2a, in daily subcutaneous doses of 2, 10, or 50 μg (total volume of 50 μl) for four consecutive days. On day 2, the mice were exposed to a lethal intranasal dose (ten times the LC50) of vesicular stomatitis virus (VSV). Data is expressed as the number of mice which survive to day 21.

FIG. 5 shows that both of the mouse-optimized interferon-alpha homologues, CH2.2 and CH9.3, were as effective or more effective than native murine interferon Mu-IFN alpha 4 in protecting mice from VSV. At the concentrations tested, human IFN-alpha 2a was nearly completely ineffective in protecting mice from the virus. Thus, the in vivo efficacy of the interferon-alpha homologues of the invention correlates remarkably well with the antiviral activities observed in the in vitro assays.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated herein by reference in their entirety for all purposes to the same extent as if each individual publication, patent, patent application, or other document were individually indicated to be incorporated by reference for all purposes.

SEQUENCES

| | Clone | |
|---|---|---|
| SEQ ID | ID | Sequence |
| SEQ ID NO:1 | 2DH12 | TGTGATCTGCCTCAGACCCACAGCCTTGGCAACAGGAGGGCCTTGATG |
| | | CTCCTGGCACAAATGGGACGAATCTCTCCTTTCTCCTGCCTGAAGGAC |
| | | AGACAAGACTTTGGATTCCCCCAGGAGGAGTTTGATGGCAACCAGTTC |
| | | CAGAAGGCTCAAGCCATCTCTGTCCTCCATGAGATGATCCAGCAGACC |
| | | TTCAATCTCTTCAGCACAAAGGATTCATCTGCTGCTTGGGAACAGACC |
| | | CTCCTAGAAAAATTTTCCACTGAACTCTACCAGCAGCTGAATGACCTG |
| | | GAAGCCTGCGTGATACAGGAGGTAGGGGTGAAAGAGACTCCCCTGATG |
| | | AATGTGGACTCCATCCTGGCTGTGAGGAAGTACTTCCAAAGAATCACT |
| | | CTTTATCTAATAGAGAGGAAATACAGCCCTTGTGCATGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:2 | 2CA3 | TGTGATCTGCCTCAGACCCACAGCCTTGGTGACAGGAGGGCCATGATA |
| | | CTCCTCGCACAAATGGGACGAATCTCTCCTTTCTCCTGCCTGAAGGAC |
| | | AGATATGATTTCGGATTCCCCCAGGAGGAGTTTGATGGCAACCAGTTC |
| | | CAGAAGGCTCAAGCCATCTCTGTCCTCCATGAGATGATCCAGCAGACC |
| | | TTCAATCTCTTCAGCACAAAGGATTCATCTGCTGCTTGGGAACAGAGC |
| | | CTCCTAGAAAAATTTTCCACTGAACTTTACCAGCAGCTGAATGAACTG |
| | | GAAGCATGTGTGATACAGGAGGTTGGGGTGGGAGAGACTCCCCTGATG |
| | | AATGGGGACTCCATCCTGGCTGTGAAGAAGTACTTCCAAAGAATCACT |
| | | CTTTATCTAATAGAGAGGAAATACAGCCCTTGTGCATGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:3 | 4AB9 | TCTGATCTGCCTCAGACCCACAGCCTTGGCAACAGGAGGGCCTTGATA |
| | | CTCCTGGCACAAATGGGACGAATCTCTCCTTTCTCCTGCCTGAAGGAC |
| | | AGACATGACTTTGGATTCCCCCGGGAGGAGTTTGATGGCAACCAGTTC |
| | | CTCCTGGCACAAATGGGACGAATCTCTCCTTTCTCCTGCCTGAAGCAC |
| | | AGACATGACTTTCGATTCCCCCGGGAGGAGTTTGATGGCAACCAGTTC |
| | | CAGAAGGCTCAAGCCATCTCTGTCCTCCATGAGATGATGCAGCAGACC |
| | | TTCAATCTCTTCAGCACAAAGAACTCATCTGCTGCTTGGGATGAGACC |
| | | CTCCTAGAAAAATTTTCCACTGAACTTTACCAGCAACTGAATGAACTG |
| | | GAAGCATGTGTGATACAGGAGGTTGGGGTGGAAGAGACTCCCCTGATG |
| | | AATGAGGACTCCATCCTGGCTGTGAAGAAATACTTCCAAAGAATCACT |
| | | CTTTATCTGACAGAGAAGAAGTATACCCCTTGTTCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:4 | 2DA4 | TGTCATCTGCCTCAGACCCACAGCCTTGGTAACAGGAGGGCCTTGATG |
| | | CTCCTGGCACAAATGGGAAGAATCTCTCCTTTCTCCTGCCTGAAGGAC |
| | | AGACAAGACTTTGGATTCCCCCAGGAGGAGTTTGATAGCAACCAGTTC |
| | | CAGAAGGCTCAAGCCATCTCTGTCCTCCATCAGATCATGCAGCAGACC |
| | | TTCAATCTCTTCAGCACAAAGGACTCATCTGCTGCTTGGGATGAGACC |
| | | CTCCTAGAAAAATTTTCCACTGAACTCTACCAGCAGCTGAATGACCTG |
| | | GAAGCCTGCGTGATACAGGAGGTTGGGGTCGAAGAGACCCCCCTGATG |
| | | AATGTGGACTCCATCCTGGCTGTCAGGAAGTACTTCCAAAGAATCACT |
| | | CTTTATCTAATAGAGAGGAAATACACCCCTTGTGCATGGGAGGTTGTC |
| | | AGAGCAGAAATCATGAGATCTTTCTCTTTTTCAACAAACTTGCAAAAA |
| | | AGATTAAGGAGGAAGCAA |
| SEQ ID NO:5 | 3DA11 | TCTGATCTGCCTCAGACCCACAGCCTTCGTAACAGGAGGGCCTTGGTA |
| | | CTCCTGGCACAAATGGGAAGAATCTCTCCTTTCTCCTGCCTGAAGCAC |
| | | AGATATGATTTCGGATTCCCCCAGGAGCAGTTTGATGGCAACCAGTTC |
| | | CAGAAGGCTCAAGCCATCTCTGTCCTCCATGAGATGATCCAGCAGACC |
| | | TTCAATCTCTTCACCACAAAGCATTCATCTGCTGCTTGGGATGAGACC |
| | | CTCCTAGAAAAATTTTCCACTGAACTTTACCAGCAGCTGAATGACCTG |
| | | GAAGCCTGCGTGATACAGGAGGTTGGGGTGGAAGAGACCCCCCTGATC |
| | | AATGAGGACTCCATCCTGGCTGTGAAGAAATACTTCCAAAGAATCACT |
| | | CTTTATCTAATAGAGAGGAAATACAGCCCTTGTGCATGGGAGGTTGTC |
| | | AGAGCAGAAATCATCAGATCTTTCTCTTTTTCAACAAACTTGCAAAAA |
| | | AGATTAAGGAGGAAGGAA |
| SEQ ID NO:6 | 2DB11 | TGTGATCTGCCTCAGACCCACAGCCTTGGTAACAGGAGGGCCTTGATG |
| | | CTCCTGGCACAAATGGGAACAATCTCTCCTTTCTCCTGCCTGAAGGAC |
| | | AGATATGATTTCGGATTCCCCCAGGAGGAGTTTGATGGCAACCAGTTC |
| | | CAGAAGGCTCAAGCCATCTCTGTCCTCCATGAGATGATCCAGCAGACC |
| | | TTCAATCTCTTCAGCACAAAGGATTCATCTGCTGCTTGGGATGAGACC |
| | | CTCCTAGAAAAATTTTCCACTGAACTTTACCAGCAGCTGAATGACTTG |
| | | GAAGCCTGTGTGATACAGGAGGTTGGCGTGGAAGAGACTCCCCTGATG |
| | | AATGTGGACTCCATCCTGGCTGTGAGGAAGTACTTCCAAAGAATCACT |
| | | CTTTATCTAATAGAGAGGAAATACAGCCCTTGTGCATGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:7 | 2CA5 | TGTGATCTGCCTCAGACCCACAGCCTTGGTAACAGGAGGGCCTTGATA |
| | | CTCCTGGCACAAATCGGACGAATCTCTCCTTTCTCCTGCCTGAAGGAC |
| | | AGACAAGACTTTGGATTCCCCCAGGAGGAGTTTGATGGCAACCGGTTC |
| | | CAGAAGGCTCAAGCCATCTCTGTCCTCCATGAGATGATCCAGCAGACC |
| | | TTCAATCTCTTCAGCACAAAGAACTCATCTGCTGCTTGGGAACAGAGC |
| | | CTCCTAGAAAAATTTTCCACTGAACTCTACCAGCAGCTGAATGACCTG |
| | | GAAGCCTGCGTGATACAGGACGTTCGGCTGGAAGAGACCCCCCTGATG |
| | | AATGAGGACTCCATCCTGGCTGTGAAGAAATACTTCCAAAGAATCACT |
| | | CTTTATCTAATAGAGAGGAAATACAGCCCTTGTGCATGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:8 | 2G6 | TGTGATCTGCCTCAGACCCACAGCCTTGGTPACAGGAGGCCCTTGATA |
| | | CTCCTGGCACAAATGGGAAGAATCTCTCCTTTCTCCTGCCTGAAGGAC |
| | | AGACATGACTTTGGATTCCCCCAGGAGGAGTTTGATGGCAACCAGTTC |
| | | CAGAAGGCTCAAGCCATCTCTGTCCTCCATGAGATGATCCAGCAGACC |
| | | TTCAATCTCTTCAGCACAAAGGACTCATCTGCTACTTGGGAACAGAGC |
| | | CTCCTAGAAAAATTTTCCACTGAACTTAACCAGCAGCTGAATGACCTG |
| | | GAAGCCTGCGTGATACAGGAGGTTGGGGTGGAAGAGACTCCCCTGATG |
| | | AATGTGGACCCCATCCTGGCTGTGAAGAAATACTTCCAAAGAATCACT |
| | | CTCTATCTGACAGAGAAGAAATACAGCCCTTGTGCCTGCGAGGTTCTC |
| | | AGAGCAGAAATCATGAGATCTTTCTCTTTTTCAACAAACTTGCAAAAA |
| | | AGATTAAGGAGGAAGGAA |
| SEQ ID NO:9 | 3AH7 | TGTGATCTGCCTCAGACCCACAGCCTTGGTAACAGGAGGGCCTTGATA |
| | | CTCCTGGCACAAATGCGAAGAATCTCTCCTTTCTCCTGCCTGAAGGAC |
| | | AGACATGACTTTGGATTCCCCCAGGAGGAGTTTGATAGCAACCAGTTC |
| | | CAGAAGGCTCAAGCCATCTCTGTCCTCCATGAGATGATCCAGCAGACC |
| | | TTCAATCTCTTCAGCACAAAGGATTCATCTGCTGCTTGGGAACAGAGC |
| | | CTCCTAGAAAAATTTTCCACTGAACTTCACCAGCAACTGAATGAACTC |
| | | GAAGCATGTGTAGTACAGGAGGTTGGGGTGGAAGAGACTCCCCTGATG |
| | | AATGAGGACTCCATCCTGGCTGTGAAGAAATACCTCCAAAGAATCACT |
| | | CTTTATCTGACACAGAAGAAGTATAGCCCTTGTGCATGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:10 | 2G5 | TGTGATCTGCCTCACACCCACAGCCTTGGTAACAGGAGCGCCTTGATG |
| | | CTCCTGGCACAAATGGGAAGAATCTCTCCTTTCTCCTGCCTGAAGGAC |
| | | AGACAAGACTTTGGATTCCCCCAGGAGGAGTTTGATGGCAACCAGTTC |
| | | CAGAACGCTCAAGCCATCTCTGTCCTCCATGAGATGATCCAGCAGACC |
| | | TTCAATCTCTTCAGCACAAAGGATTCATCTGCTGCTTGGGAACAGAGC |
| | | CTCCTAGAAAAATTTTCCACTGAACTCTACCAGCAGCTGAATGACCTG |
| | | GAAGCCTGCGTGATACAGGAGGTTGGGGTGGAAGAGACCCCCCTGATG |
| | | AATGTGGACTCCATCCTGGCTGTGAGGAAGTACTTCCAAAGAATCACT |
| | | CTTTATCTAATAGAGAGGAAATACAGCCCTTGTGCATGGGAGGTTGTC |
| | | AGAGCAGAAATCATGAGATCTTTCTCTTTTTCAACAAACTTGCAAAAA |
| | | AGATTAAGGAGGAAGCAA |
| SEQ ID NO:11 | 2BA8 | TGTGATCTGCCTCAGACCCACAGCCTTGGTAACAGGAGGGCCCTGATA |
| | | CTCCTGGCACAAATGGGACGAATCTCTCCTTTCTCCTGCCTGAAGGAC |
| | | AGATATGATTTCGGATTCCCCCAGGAGGAGTTTGATGGCAACCAGTTC |
| | | CAGAAGGCTCAAGCCATCTCTGTCCTCCATGAGATGATCCAGCAGACC |
| | | TTCAATCTCTTCAGCACAAAGGATTCATCTGCTGCTTGGGAACAGAGC |
| | | CTCCTAGAAAAATTTTCCACTGAACTTTACCAGCAGCTGAATGACCTG |
| | | GAAGCCTGCGTGATACAGGAGGTTGGGGTGGAAGAGACCCCCCTAATG |
| | | AATGTGGACTCCATCCTGGCTGTGAGGAAGTACTTCCAAAGAATCACT |
| | | CTTTATCTAATAGAGAGGAAATACAGCCCTTGTGCATGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:12 | 1F3 | TGTGATCTGCCTCAGACCCACAGCCTTCGTAACAGCAGGGCCTTGATA |
| | | CTCCTGGGACAAATGGGAAGAATCTCTCATTTCTCCTGCCTGAAGGAC |
| | | AGACATGACTTTGGATTCCCCCAGGAGGAGTTTGATGGCAACCAGTTC |
| | | CAGAAGGCTCAAGCCATCTCTGTCCTCCATGAGATGATCCAGCAGACC |
| | | TTCAACCTCTTCAGCACAAACGACTCATCTGTTGCTTGGGATGAGAGG |
| | | CTTCTAGACAAACTCTATACTCAACTTTACCAGCAGCTGAATGACCTG |
| | | GAAGCCTGTGTGATCCAGGAGGTGTGGGTGGGAGCGACTCCCCTGATC |
| | | AATGACGACTCCATCCTGGCTGTGAGAAAATACTTCCAAAGAATCACT |
| | | CTCTATCTGACAGAGAAGAAATACAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:13 | 4BE10 | TGTGATCTGCCTCAGACCCACAGCCTTGGTAACAGGAGGCCCTTGATA |
| | | CTCCTGGCACAGATGGGACGAATCTCTCCTTTCTCCTGCCTGAAGGAC |
| | | AGATATGATTTCGGATTCCCCCAGGAGGAGTTTGATGGCAACCAGTTC |
| | | CAGAAGGCTCAAGCCATCTCTGTCCTCCATGAGATAATGCAGCAGACC |
| | | TTCAATCTCTTCAGCACAAAGAACTCATCTGCTCCTTGGGATGAGACC |
| | | CTCCTAGAAAAATTTTCCACTGAACTTTACCAGCAACTGAATGAACTG |
| | | GAAGCATGTGTGATACAGGGGGTTGCGGTGGAAGAGACTCCCCTGATG |
| | | AATGAGGACTCCATCTTGGCTGTGAGGAAATACTTCCAAAGAATCACT |
| | | CTTTATCTGACAGAGAAGAAGTATAGCCCTTGTTCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:14 | 2DD9 | TGTGATCTGCCTCAGACCCACAGCCTTGGTAACAGGAGGGCCTTGATG |
| | | CTCCTGGCACAAATGGGAAGAATCTCCCCTTTCTCCTGCCTGAAGGAC |
| | | AGATATGATTTCGGATTCCCCCAGGAGGAGTTTGATGGCAACCAGTTC |
| | | CAGAAGGCTCAAGCCATCTCTGTCCTCCATGAGATGATCCAGCAGACC |
| | | TTCAATCTCTTCAGCACAAAGGATTCATCTGCTCCTTGGGAACACAGC |
| | | CTCCTAGAAAAATTTTCCACTGGACTCTACCAGCAGCTGAATGACCTG |
| | | GAAGCCTGCGTGATACACGAGGTTGGGGTGGAAGAGACCCCCCTGATG |
| | | AATGAGGACTCCATCCTGGCTGTGAGGAAATACTTCCAAAGAATCACT |
| | | CTTTATCTGACAGAGAAGAAGTATAGCCCTTGTTCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:15 | 3CA1 | TGTGATCTGCCTCAGACCCACAGCCTTGGCAACAGGAGGGCCTTGATA |
| | | CTCCTGGCACAAATGGGAACAATCTCTCCTTTCTCCTGCCTGAAGGAC |
| | | AGACATGACTTTGGATTACCCCAGGAGGAGTTTGATGGCAACCAGTTC |
| | | CAGAAGGCTCAAGCCATCTCTGTCCTCCATGAGATGATCCAGCAGACC |
| | | TTCAATCTCTTCAGCACAAAGAACTCATCTGCTGCTTGGGATGAGACC |
| | | CTCCTAGAAAAATTTTCCACTGAACTTTACCAGCAACTGAATAACCTG |
| | | GAAGCATGTGTGATACAGGAGGTTGGGATCGAAGAGACTCCCCTGATG |
| | | AATGTGGACTCCATCCTGGCTGTGAAGAAATACTTCCAAAGAATCACT |
| | | CTTTATCTGACACAGAAGAAGTATAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:16 | 2F8 | TGTGATCTGCCTCAGACCCACAGCCTTGGTAACAGGAGGGCCTTGATA |
| | | CTCCTGGCACAAATGGGACGAATCTCTCCTTTCTCCTGCCTGAAGGAC |
| | | AGATATGATTTCGGATTCCCCCAGGACGAGTTTGATGGCAACCAGTTC |
| | | CACAAGGCTCAAGCCATCTCTGTCCTCCATGAGATGATGCAGCACACC |
| | | TTCAATCTCTTCAGCACAAAGAACTCATCTGCTGCTTGGGATGAGACC |
| | | CTCCTAGAAAAATTTTCCACTGAACTTTACCAGCAACTGAATGAACTG |
| | | GAAGCATGTGTGATACAGGAGGTTGGGGTGGAAGAGACTCCCCTGATG |
| | | AATGAGGACTCCATCCTGGCTGTGAAGAAATACTTCCAAAGAATCACT |
| | | CTTTATCTGACAGAGAAGAAGTATAGCCCTTGTTCCTGGGACGTTGTC |
| | | AGAGCAGAAATCATGAGATCTTTCTCTTTTTCAACAAACTTGCAAAAA |
| | | AGATTAGGAGGAGGAA |
| SEQ ID NO:17 | 6CG3 | TGTGATCTGCCTCAGACCCACAGCCTTGGTAACAAGAGGGCCATGATG |
| | | CTCCTGGCACAAATGGGAAGAACCTCTCCTTTCTCCTGTCTGAAGGAC |
| | | ACACATGACTTTGGATTCCCCCAGGAGGAGTTTGATGGCAACCAGTTC |
| | | CACAGGGCTCAAGCCATCTTTGTCCTCCATCAGATGATCCAGCAGACC |
| | | TTCAATTTCTTCAGCACAAACGACTCATCTGCTGCTTGGGAACAGACC |
| | | CTCCTAGAAAAATTTTCCACTGAACTTAACCAGCAGCTGAATGACCTG |
| | | GAAGCCTGCGTGATACAGGAAGTTGGGGTGGAAGAGACTCCCCTGATG |
| | | AATGAGGACTCCATCCTGGCTGTGAAGAAATACTTCCAAAGAATCACT |
| | | CTTTATCTCACAGAGAAGAAATACAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:18 | 3CG7 | TGTGATCTGCCTCAGACCCACAGCCTTGGTAACAGTAGGGCCTTGATG |
| | | CTCCTGGCACAAATCGGAAGAATCTCCCCTTTCTCCTGCCTGAAGGAC |
| | | AGACATGATTTCGGATTCCCCCAGGAGGACTTTGATGGCAACCAGTTC |
| | | CAGAAGGCTCAAGCCATCTCTGCCTTCCATGAGATGATCCAGCAGACC |
| | | TTCAATCTCTTCAGCACAAAGGATTCATCTGCTGCTTGGGAACAGAAC |
| | | CTCCTAGAAAAATTTTCCACTGAACTTTACCAGCAACTGAATAACCTG |
| | | GAAGCATGTCTGATACAGGACGTTGGGATGGAAGAGACTCCCCTGATG |
| | | AATCTGGACTCCATCCTGGCTGTGAGGAAGTACTTCCAAAGAATCACT |
| | | CTTTATCTAATAGAGAGGAAATACAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:19 | 1D3 | TGTGATCTGCCTCAGACCCACAGCCTTCGTAACAGGAGGGCCTTGATA |
| | | CTCCTGGCACAAATGGGAAGAATCTCTCATTTCTCCTGCCTGAAGGAC |
| | | AGACATGATTTCGGATTCCCCCAGGAGGAGTTTGATGGCCACCAGTTC |
| | | CAGAAGACTCAAGCCATCTCTGTCCTCCATGAGATGATCCAGCAGACC |
| | | TTCAATCTCTTCAGCACAAAGGACTCATCTGCTGCTTGGGAACAGAGC |
| | | CTCCTAGAAAAATTTTCCACTGAACTTTACCAGCAACTGAATAACCTG |
| | | GAAGCATGTGTGATACAGGAGGTTGGCGTGGAAGAGACTCCCCTGATG |
| | | AATGAGGACTCCATCCTGGCTGTGAAGAAATACTTCCAAAGAATCACT |
| | | CTTTATCTGATGGAGAAGAAATACAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:20 | 2G4 | TGTGATCTGCCTCAGACCCACAGCCTTGGTAACAGGAGGGCCATGATG |
| | | CTCCTGGCACAAATGAGCAGAATCTCTCCTTCCTCCTGTCTGATGGAC |
| | | AGACATGACTTTGAATTTCCCCAGGAGCAATTTCATGATAAACAGTTC |
| | | CAGAAGGCTCCAGCCATCTCTGTCCTCCATGAGGTGATTCAGCAGACC |
| | | TTCAATCTCTTCAGCACAGAGGACTCATCTGCTGCTTGGGAACAGACC |
| | | CTCCTAGAAAAATTTTCCACTGAACTTTACCAGCAACTGAATGACCTG |
| | | GAAGCATGTGTGATGCAGGAGGAGAGGGTGGGAGAAACTCCCCTGATG |
| | | AATGCGGACTCCATCTTGGCTGTGAGGAAATACTTCCAAAGAATCACT |
| | | CTTTATCTGACAAAGAAGAAGTATAGCCCTTGTTCCTGGGAGGTTGTC |
| | | AGAGCAGAAATCATGAGATCTTTCTCTTTTTCAACAAACTTGCAAAAA |
| | | ACATTAAGCAGGAAGGAA |
| SEQ ID NO:21 | 1A1 | TGTGATCTGCCTCAGACCCACAGCCTTGGTAACAGGAGGGCCTTGATA |
| | | CTCCTGCCACAAATGGGAAGAATCTCTCATTTCTCCTGCCTGAAGGAC |
| | | AGATATGATTTCGGATTCCCCCAGGAGGTGTTTGATGGCAACCAGTTC |
| | | CAGAAGGCCCAAGCCATCTCTGCCTTCCATGAGATGATGCAGCAGACC |
| | | TTCAATCTCTTCAGCACAGAGCACTCATCTGCTGCTTGGGAACAGAGC |
| | | CTCCTAGAAAAATTTTCCACTGAACTTCACCAGCAACTGAATGACCTG |
| | | GAAGCCTGTGTGATACAGGAGGTTGGGGTGGAAGAGACTCCCCTGATG |
| | | AATGAGGACTCCATCCTGGCTGTGAGGAAATACTTTCAAAGAATCACT |
| | | CTTTATCTAATGGAGAAGAAATACAGCCCTTGTGCCTGGGAGGTTGTC |
| | | AGAGCAGAAATCATCAGATCTTTCTCTTTTTCAACAAACTTGCAAAAA |
| | | AGATTAAGGAGCAAGGAA |
| SEQ ID NO:22 | 1D1G | TGTGATCTGCCTCAGACCCACAGCCTTGCTAACAGGAGGGCCTTCATA |
| | | CTCCTGGCACAAATGGGAAGAATCTCTCATTTCTCCTGCCTGAAGGAC |
| | | AGACATGATTTCGGATTCCCCCAGGAGGAGTTTGATGGCCACCAGTTC |
| | | CAGAAGACTCAAGCCATCTCTGTCCTCCATGAGATGATCCAGCAGACC |
| | | TTCAATCTCTTCAGCACAAAGGACTCATCTGCTGCTTGGGAACAGAGC |
| | | CTCCTAGAAAAATTTTCCACTGAACTTTACCAGCAACTGAATGACCTG |
| | | GAAGCATGTGTGATACAGGAGGTTGGGGTGGAAGACACTCCCCTGATG |
| | | AATGAGGACTCCATCCTGGCTGTGAAGAAATACTTCAAAGAAGTCACT |
| | | CTTTATCTGATGGAGAAGAAATACAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:23 | 1F6 | TGTGATCTGCCTCAGACCCACAGCCTTGGTAACAGGAGGACTTTGATG |
| | | ATAATGGCACAAATGGGAAGAATCTCTCCTTTCTCCTGCCTGAAGGAC |
| | | AGACATGACTTTGGATTTCCCCAGGAGGAGTTTGATGGCAACCAGTTC |
| | | CAGAACGCTCAAGCCATCTCTGTCCTCCATGAGATGATCCAGCAGACC |
| | | TTCAATCTCTTCAGCACAAAGGACTCATCTGCTACTTGGGAACAGAGC |
| | | CTCCTAGAAAAATTTTCCACTGAACTTAACCAGCAGCTGAATGACCTG |
| | | GAAGCCTGCCTGATACAGGAGGCTGGGGTGGAAGAGACTCCCCTGATG |
| | | AATGTGGACTCCATCCTGGCTGTGAAGAAATACTTCCAAAGAATCACT |
| | | CTTTATCTAACAGAGAAGAAATACAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:24 | 2A10 | TGTGATCTGCCTCAGACCCACAGCCTTGGTAACAGGAGGGCCTTGATA |
| | | CTCCTGGCACAAATGGGAAGAATCTCTCATTTCTCCTGCCTGAAGGAC |
| | | AGATATGATTTCGGATTCCCCCAGGAGGTGTTTGATGGCAACCAGTTC |
| | | CAGAAGGCTCAAGCCATCTCTGCCTTCCATGAGATGATCCAGCAGACC |
| | | TTCAATCTCTTCAGCACAAAGGACTCATCTGCTACTTGGGAACAGAGC |
| | | GAAGCATGTGTGATACAGGAGGTTGGGGTGGAAGAGACTCCCCTGATG |
| | | AATGAGGACTCCATCCTGGCTGTGAGGAAATACTTTCAAAGAATCACT |
| | | CTTTATCTGATGCAGAAGAAATACAGCCCTTGTGCCTGGGAGCTTGTC |
| | | AGAGCAGAAATCATGAGATCTTTCTCTTTTTCAACAAACTTGCAAAAA |
| | | AGATTAAGGAGGAAGGAA |
| SEQ ID NO:25 | 2C3 | TGTGATCTGCCTCAGACCCACAGCCTTGGTAACAGGAGGGCCTTGATA |
| | | CTCCTGGCACAAATGGGAAGAATCTCTCCTTTCTCCTGCCTGAAGGAC |
| | | AGACATGACTTTGGATTTCCTCAGGAGGAGTTTGATGGCAACCAGTCC |
| | | CAGAAGGCTCAAGCCATCTCTGTCCTCCATGAGATGATCCAGCAGACC |
| | | TTCAATCTCTTCAGCACAAAGGACTCATCTGATACTTGGGATGCGACC |
| | | CTTTTAGAAAAATTTTCCACTGAACTTAACCAGCACCTGAATGACCTG |
| | | GAAGCCTGCGTGATACAGGAGGTTGGGGTGGAAGAGACCCCCCTGATG |
| | | AATGTGGACTCCATCCTGGCTGTGAAGAAATACTTCCAAAGAATCACT |
| | | CTTTATCTGACAGAGAAGAAATACAGCCCTTGTGCCTGGGAGGTTGTC |
| | | ACAGCACAAATCATGAGATCTTTCTCTTTTTCAACAAACTTGCAAAAA |
| | | AGATTAAGGAGGAAGGAA |
| SEQ ID NO:26 | 2D1 | TGTGATCTGCCTCAGACCCACAGCCTTGGTAACAGGAGGGCCTTGATA |
| | | CTCCTGGCACAAATGCGACGAATCTCTCCTTTCTCCTGCCTGAAGGAC |
| | | AGACAAGACTTTGGATTCCCCCAGCACGAGTTTCATGGCAACCGGTTC |
| | | CAGAAGGCTCAAGCCATCTCTGTCCTCCATGAGATGATCCAGCAGACC |
| | | TTCAATCTCTTCAGCACAAAGAACTCATCTGCTGCTTGGGAACAGAGC |
| | | CTCCTAGAAAAATTTTCCACTGAACTCTACCAGCACCTGAATGACCTG |
| | | GAAGCCTGCGTGATACAGGAGGTTGGGGTGGAAGAGACCCCCCTGATG |
| | | AATGAGGACTCCATCCTGGCTGTGAAGAAATACTTCCAAAGAATCACT |
| | | CTTTATCTAATAGAGAGGAAATACAGCCCTTGTGCATGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:27 | 2D10 | TGTGATCTGCCTCAGACCCACAGCCTTGGTAACAGGAGGGCCTTGATA |
| | | CTCCTGGCACAAATGGGAAGAGTCTCTCCTTTCTCCTGCCTGAAGGAC |
| | | ACACATGACTTTGGATTCCCCCAGGAGGAGTTTGATGGCAACCAGTTC |
| | | CAGAAGGCTCAAGCCATCTCTGCCTTCCATGAGATGATCCAGCAGACC |
| | | TTCAATCTCTTCAGCACAAAGGACTCATCTGCTACTTGGGAACAGAGC |
| | | CTCCTAGAAAAATTTTCCACTGAACTTTACCAGCAACTGAATAACCTG |
| | | CAAGCCTGCGTGATACAGGAGGTTGGGGTGGAAGAGACTCCCCTGATG |
| | | AATGTGGACTCCATCCTGGCTGTGAAGAAATACTTCCGAAGAATCACT |
| | | CTCTATCTGACAGAGAAGAAATACAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:28 | 2D7 | TGTGATCTGCCTCAGACCCACAGCCTTGGTAACAGGCGGGCCTTGATA |
| | | CTCCTGGCACAAATGGGAAGAATCTCTCCTTTCTCCTGTCTGAAGGAC |
| | | AGACATGACTTCAGATTTCCCCAGGAGGAGTTTGATGGCAACCAGTTC |
| | | CAGAAGGCTCAAGCCATCTCTGTCCTCCATGAGATGATCCAGCAGACC |
| | | TTCAATCTCTTCAGCACAAAGGACTCATCTGCTACTTGGGAACAGAGC |
| | | CTCCTAGAAAAATTTTCCACTGAACTTTACCAGCAACTGAATAACCTG |
| | | GAAGCTTGCGTGATACAGGAGGTTGGGGTGGAAGAGACTCCCCTGATG |
| | | AATGTGGACTCTATCCTGGCTGTGAAGATAAACTTCCAAAGAATCACT |
| | | CTTTATCTGACAGAGAGGAAATACAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:29 | 2D9 | TGTGATCTGCCTCAGACCCACAGCCTTGGTAACAGGAGGGCCTTGATA |
| | | CTCCTGGCACAAATGGGAAGAATCTCTCCTTTCTCCTGCCTGAAGGAC |
| | | AGACATGACTTTGGATTCCCCCAGGAGGAGTTTCATGGCAACCAGTTC |
| | | CAGAAGGCTCAAGCCATCTCTGTCCTCCATGAGATGATCCAGCAGACT |
| | | TTCAATCTCTTCAGCACAAAGGACTCATCTGCTACTTGGGAACAGAGC |
| | | CTCCTAGAAAATTTTCCACTGAACTTAACCAGCAGCTGAATGACCTG |
| | | GAAGCCTGCGTGATACAGGAGGTTGGGGTGGAAGAGACTCCCCTGGTG |
| | | AATGTGGACTCCATCCTGGCTGTGAAGAAATACTTCCAAAGAATCACT |
| | | CTTTATCTGACAGAGAAGAAATACAGCCCTTGTGCCTGGGAGGTTGTC |
| | | AGAGCAGAAATCATGAGATCTTTCTCTTTTTCAACMACTTGCAAAAA |
| | | AGATTAAGGAGGAAGGAA |
| SEQ ID NO:30 | 2DA2 | TCTGATCTGCCTCAGACCCACAGCCTTGGTAACAGGAGGCCCTTGATA |
| | | CTCCTGGCACAAATGGGAAGAATCTCTCCTTTCTCCTGCCTGAAGGAC |
| | | AGACAGCACTTCGGATTCCCCCAGGAGGAGTTTGATGGCAACCAGTTC |
| | | CAGAAGGCTCAAGCCATCTCTGTCCTCCATGAGATGATGCAGCAGACC |
| | | TTCAATCTCTTCAGCACAAAGAACTCATCTGCTGCTTGGGAACAGAGC |
| | | CTCCTAGAAAAATTTTCCACTGAACTCCACCAGCAACTGAATGAACTG |
| | | GAAGCATGTGTGATACAGGAGGTTGGCGTGGAAGAGACTCCCCTGATG |
| | | AATGTGGACTCCATCCTGGCTGTGAAGAAATACTTCCAAAGAATCACT |
| | | CTTTATCTAATAGAGACCAAATACAGCCCTTGTGCATGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:31 | 2DH9 | TGTGATCTCCCTCAGACCCACAGCCCTGGTAACAGGAGGGCCTTGATG |
| | | CTCCTGGCACAAATGGGACGAATCTCTCCTTTCTCCTGCCTGAAGGAC |
| | | AGATATGATTTCGCATTCCCCCAGGGGGAGTTTGATCGCAACCAGTTC |
| | | CAGAAGGCTCAAGCCATCTCTGTCCTCCATGAGATGATGCAGCAGACC |
| | | TTCAATCTCTTCAGCACAAAGGATTCATCTGCTGCTTGGGAACAGAGC |
| | | CTCCTAGAAAAATTTTCCACTGAACTCTACCGGCAGCTGAATGACCTG |
| | | GAAGCCTGTGTGATACAGGAGGTTGGGGTGGAACAGACCCCCCTGATG |
| | | AATGTGGACTCCATCCTGGCTGTGAGGAAGTACTTCCAAAGAATCACT |
| | | CTTTATCTGACAGAGAAGAAGCATAGCCCTTGTTCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:32 | 2G11 | TGTGATCTGCCTCAGACCCACAGCCTTGGTAACAGGAGGCCCTTGATA |
| | | CTCCTGGCACAAATGGGAAGAATCTCTCCTTTCTCCTGCCTGAAGGAC |
| | | AGACATGACTTTGGACTTCCCCAGGAGGAGTTTGATGGCAACCAGTTC |
| | | CAGAAGACTCAAGCCATCTCTGTCCTCCATGAGATGATCCAGCAGACC |
| | | TTCAATCTCTTCACCACAAAGGACTCATCTGATACTTGGGAACAGAGC |
| | | CTCCTAGAAAAATTCTACATTGAACTTTTCCAGCAGCTGAATGACCTG |
| | | GAAGCCTGCGTGATACAGGAGGTTGGGGTGGAAGAGACTCCCCTGATG |
| | | AATGTGGACTCCATCCTGGCTGTCAGAAAATACTTCCAAAGAATCACT |
| | | CTTTATCTGACAGAGGAGAAATACAGCCCTTGTGCCTGGGAGGTTGTC |
| | | AGAGCACAAATCATGAGATCTTTCTCTTTTTCAACAAACTTGCAAAAA |
| | | ACATTAAGGAGGAAGGAA |
| SEQ ID NO:33 | 2G12 | TGTGATCTGCCTCAGACCCACAGCCTTGGTAACAGGAGCACTTTGATG |
| | | CTCATGGCACAAATGAGGAGAATCTCTCCTTTCCCCCGCCTGAAGGAC |
| | | AGATATGATTTCGGATTCCCCCAGGAGGTGTTTGATGGCAACCAGTTC |
| | | CAGAAGGCTCAAGCTATCTTCCTTTTCCATGAGNIGATGCAGCAGACC |
| | | TTCAATCTCTTCAGCACAAAGAACTCATCTGCTGCTTGGGATGAGACC |
| | | CTCCTAGACAAATTCTACACTGAACTCTACCAGCAGCTGAATGACTTG |
| | | GAAGCCTGTGTGATGCAGGAGGGGAGGGTGGGAGAAACTCCCCTGATG |
| | | AATGCGGACTCCATCTTGGCTGTGAAGAAATACTTCCGAAGAATCACT |
| | | CTCTATCTGACAGAGAAGAAATACACCCCTTGTGCCTGGGAGGCTGTC |
| | | AGAGCAGAAATCATGAGATCTTTCTCTTTTTCAACAAACTTGCAAAAA |
| | | ACATTAAGGAGGAAGGAA |
| SEQ ID NO:34 | 2H9 | TGTGATCTGCCTCAGACCCACAGCCTTGGTAACAGGAGGGCCTTGATA |
| | | CTCCTGGCACAAATGGGAAGAATCTCTCCTTTCTCCTGCCTGAAGGAC |
| | | AGACATGACTTTGGATTCCCCCAGGAGGAGTTTGATGGCAACCAGTTC |
| | | CAGAAGGCTCAAGCCATCTCTGTCCTCCATGAGATGATCCAGCAGACC |
| | | TTCAATCTCTTCAGCACAAAGGACTCATCTGCTACTTGGGAACAGAGC |
| | | CTCCTAGAAAAATTTTCCACTGAACTTAACCACCAGCTGAATGACCTA |
| | | GAAGCCTGTGTGACACAGGAGGTTGGGGTGGAAGAGACTCCCCTGATG |
| | | AATCAGGACTCTATCCTCGCTGTGAAGAAATACTTCCAAAGAATCACT |
| | | CTTTATCTGACAGAGAAGAAATACAGCCCTTGTGCCTGGGACGTTGTC |
| | | AGAGCAGAAATCATGAGATCTTTCTCTTTTTCAACAAACTTGCAAAAA |
| | | ACATTAAGGAGGAAGGAA |
| SEQ ID NO:35 | 6BC11 | TGTGATCTGCCTCACACCCACAGCCTTGGTAACAGGAGGGCCTTGATA |
| | | CTCCTGGCACAAATGGGAAGAATCTCTCCTTTCTCCTGCCTGAAGGAC |
| | | AGATATGATTTCGGATTCCCCCAGGAGGAGTTTGATGGCAACCAGCTC |
| | | CAGAAGGCTCAAGCCATCTCTGTCCTCCATGAGATGATCCAGCAGACC |
| | | TTCAATCTCTTCAGCACAAAGGATTCATCTGCTCCTTGGGAACAGAGC |
| | | CTCCTAGAAAAATTTTCCACTGAACTTAACCAGCAGCTGAATGACCTG |
| | | GAAGCCTGCGTGATACAGGAGGTTGGAGTGGAAGAGACTCCCCTGATG |
| | | AATGTGGACTCCATCCTGGCTGTGAAGAAATACTTCCAAAGAATCACT |
| | | CTTTATCTCACAGAGAGGAAATACAGCCCTTGTGCCTGGGAGGTTGTC |
| | | AGAGCAGAAATCATGAGATCTTTCTCTTTTTCAACAAACTTCCAAAAA |
| | | AGATTAACGAGGAAGGAA |
| SEQ ID NO:36 | 2DH12 | CDLPQTHSLGNRRALMLLAQMGRISPFSCLKDRQDFGFPQEEFDGNQF |
| | | QKAQAISVLHEMIQQTFNLFSTKDSSAAWEQTLLEKFSTELYQQLNDL |
| | | EACVIQEVGVKETPLMNVDSILAVRKYFQRITLYLTERKYSPCAWEVV |
| | | RAEIMRSFSFSTNLQKRLRRKE |
| SEQ ID NO:37 | 2CA3 | CDLPQTHSLGDRRAMTLLAQMGRISPFSCLKDRYDFGFPQEEFDGNQF |
| | | QKAQAISVLHEMIQQTFNLFSTKDSSAAWEQSLLEKFSTELYQQLNEL |
| | | EACVIQEVGVGETPLMNGDSTLAVKKYFQRITLYLIERKYSPCAWEVV |
| | | RAEIMRSFSFSTNLQKRLRRKE |
| SEQ ID NO:38 | 4AB9 | CDLPQTHSLGNRRALILLAQMGRTSPFSCLKDRHDFGFPREEFDGNQF |
| | | QKAQAISVLHEMIQQTFNLFSTKNSSAAWDETLLEKFSTELYQQLNEL |
| | | EACVTQEVCVEETPLMNVDSILAVKKYFQRITLYLTEKKYSPCSWEVV |
| | | RAEIMRSFSFSTNLQKRLRRKE |
| SEQ ID NO:39 | 2DA4 | CDLPQTHSLGNRRALMLLAQMGRISPFSCLKDRQDFGFPQEEFDSNQF |
| | | QKAQAISVLHEMNQQTFNLFSTKDSSAAWDETLLEKFSTELYQQLNDL |
| | | EACVIQEVGVEETPLMNVDSTLAVRKYFQRITLYLIERKYSPCAWEVV |
| | | RAEIMRSFSFSTNLQKRLRRKE |
| SEQ ID NO:40 | 3DA11 | CDLPQTHSLGNRRALVLLAQMGRISPFSCLKDRYDFCFPQEEFDGNQF |
| | | QKAQAISVLHEMIQQTFNLFSTKDSSAAWDETLLEKFSTELYQQLNDL |
| | | EACVIQEVGVEETPLMNVDSILAVKKYFQRITLYLTERKYSPCAWEVV |
| | | RAEIMRSFSFSTNLQKRLRRKE |
| SEQ ID NO:41 | 2DB11 | CDLPQTHSLGNRRALMLLAQMGRTSPFSCLKDRYDFGFPQEEFDGNQF |
| | | QKAQAISVLHEMTQQTFNLFSTKDSSAAWDETLLEKFSTELYQQLNDL |
| | | EACVIQEVGVEETPLMNVDSTLAVRKYFQRITLYLIERKYSPCAWEVV |
| | | RAEIMRSFSFSTNLQKRLRRKE |
| SEQ ID NO:42 | 2CA5 | CDLPQTHSLGNRRALILLAQMGRISPFSCLKDRQDFGFPQEEFDGNRF |
| | | QKAQAISVLHEMIQQTFNLFSTKNSSAAWEQSLLEKFSTELYQQLNDL |
| | | EACVIQEVGVEETPLMNEDSILAVKKYFQRITLYLIERKYSPCAWEVV |
| | | RAEIMRSFSFSTNLQKRLRRKE |
| SEQ ID NO:43 | 2G6 | CDLPQTHSLGNRRALILLAQMGRISPFSCLKDRHDFGFPQEEFDGNQF |
| | | QKAQAISVLHEMIQQTFNLFSTKDSSATWEQSLLEKFSTELNQQLNDL |
| | | EACVIQEVGVEETPLMNVDPILAVKKYFQRITLYLTEKKYSPCAWEVV |
| | | RAETMRSFSFSTNLQKRLRRKE |
| SEQ ID NO:44 | 3AH7 | CDLPQTHSLGNRPALILLAQMRRISPFSCLKDRHDFGFPQEEFDSNQF |
| | | QKAQAISVLHEMIQQTFNLFSTKDSSAAWEQSLLEKFSTELHQQLNEL |
| | | EACVVQEVGVEETPLMNEDSILAVKKYLQRITLYLTEKKYSPCAWEVV |
| | | RAEIMRSFSFSTNLQKRLRRKE |
| SEQ ID NO:45 | 2G5 | CDLPQTHSLGNRRALMLLAQMGRISPFSCLKDRQDFGFPQEEFDGNQF |
| | | QKAQAISVLHEMIQQTFNLFSTKDSSAAWEQSLLEKFSTELYQQLNDL |
| | | EACVIQEVGVEETPLMNVNDSILAVRKYFQRITLYLIERKYSPCAWEV |
| | | RAEIMRSFSFSTNLQKRLRRKE |
| SEQ ID NO:46 | 2BA8 | CDLPQTHSLGNRRALTLLAQMGRTSPFSCLKDRYDFGFPQEEFDGNQF |
| | | QKAQAISVLHEMIQQTFNLFSTKDSSAAWEQSLLEKFSTELYQQLNDL |
| | | EACVIQEVGVEETPLMNVDSILAVRKYFQRITLYLIERKYSPCAWEVV |
| | | RAETMRSFSFSTNLQKRLRRKE |
| SEQ ID NO:47 | 1F3 | CDLPQTHSLGNRRALILLCQMGRISHFSCLKDRHDFGFPQEEFDGNQF |
| | | QKAQAISVLHEMIQQTFNLFSTKDSSVAWDERLLDKLYTELYQQLNDL |
| | | EACVMQEVWVGGTPLMNEDSILAVRKYFQRTTLYLTEKKYSPCAWEVV |
| | | RAEIMRSFSFSTNLQKRLRRKE |
| SEQ ID NO:48 | 4BE10 | CDLPQTHSLGNRRALTLLAQMGRISPFSCLKDRYDFGFPQEEFDGNQF |
| | | QKAQAISVLHEIMQQTFNLFSTKNSSAAWDETLLEKFSTELYQQLNEL |
| | | EACVIQGVGVEETPLMNEDSILAVRKYFQRTTLYLTEKKYSPCSWEVV |
| | | RAETMRSFSFSTNLQKRLRRKE |
| SEQ ID NO:49 | 2DD9 | CDLPQTHSLGNRRALMLLAQMGRISPFSCLKDRYDFGFPQEEFDGNQF |
| | | QKAQATSVLHEMTQQTFNLFSTKDSSAAWEQSLLEKFSTCLYQQLNDL |
| | | EACVTQEVGVEETPLMNEDSILAVKKYFQRITLYLTEKKYSPCSWEVV |
| | | RAEIMRSFSFSTNLQKRLRRKE |
| SEQ ID NO:50 | 3CA1 | CDLPQTHSLGNRRALTLLAQMGRISPFSCLKDRHDFGLPQEEFDGNQF |
| | | QKAQAISVLHEMIQQTFNLFSTKNSSAAWDETLLEKFSTELYQQLNNL |
| | | EACVIQEVGMEETPLMNVDSILAVKKYFQRITLYLTEKKYSPCAWEVV |
| | | RAETMRSFSFSTNLQKRLRRKE |
| SEQ ID NO:51 | 2F8 | CDLPQTHSLGNRRALILLAQMGRISPFSCLKDRYDFGFPQEEFDGNQF |
| | | QKAQAISVLHEMMQQTFNLFSTKNSSAAWDETLLEKFSTELYQQLNEL |
| | | EACVIQEVGVEETPLMNEDSILAVKKYFQRITLYLTEKKYSPCSWEVV |
| | | RAEIMRSFSFSTNLQKRLRRKE |
| SEQ ID NO:52 | 6CG3 | CDLPQTHSLGNKRAMMLLAQMGRTSPFSCLKDRHDFGFPQEEFDGNQF |
| | | QRAQAIFVLHEMIQQTFNFFSTKDSSAAWEQSLLEKFSTELNQQLNDL |
| | | EACVIQEVGVEETPLMNEDSTLAVKKYFQRITLYLTEKKYSPCAWEVV |
| | | RAEIMRSFSFSTNLQKRLRRKE |
| SEQ ID NO:53 | 3CG7 | CDLPQTHSLGNSRALMLLAQMGRISPFSCLKDRHDFGFPQEEFDGNQF |
| | | QKAQAISAFHEMIQQTFNLFSTKDSSAAWEQNLLEKFSTELYQQLNNL |
| | | EACVIQEVGMEETPLMNVDSTLAVRKYFQRITLYLIERKYSPCAWEVV |
| | | RAEIMRSFSFSTNLQKRLRRKE |
| SEQ ID NO:54 | 1D3 | CDLPQTHSLGNRRALILLAQMGRISHFSCLKDRHDFGFPQEEFDGHQF |
| | | QKTQAISVLHEMTQQTFNLFSTKDSSAAWEQSLLEKFSTELYQQLNDL |
| | | EACVIQEVGVEETPLMNEDSILAVKKYFQRITLYLMEKKYSPCAWEVV |
| | | RAEIMRSFSFSTNLQKRLRRKE |
| SEQ ID NO:55 | 2G4 | CDLPQTHSLGNREAMIMILLAQNSRISPSSCLMDRHDFEFPQEEFDDKQF |
| | | QKAPAISVLHEVIQQTFNLFSTEDSSAAWEQTLLEKFSTELYQQLNDL |
| | | EACVMQEERVGETPLMNADSILAVRKYFQRITLYLTKKKYSPCSWEVV |
| | | RAEIMRSFSFSTNLQKRLRRKE |
| SEQ ID NO:56 | 1A1 | CDLPQTHSLGNRRALILLAQMGRISHFSCLKDRYDFGFPQEVFDGNQF |
| | | QKAQATSAFHEMMQQTFNLFSTEDSSAAWEQSLLEKFSTELHQQLNDL |
| | | EACVIQEVGVEETPLMNEDSILAVRKYFQRITLYLMEKKYSPCAWEVV |
| | | RAEIMRSFSFSTNLQKRLRRKE |
| SEQ ID NO:57 | 1D10 | CDLPQTHSLGNRRALILLAQMGRTSPFSCLKDRHDFRFPQEEFDGNQL |
| | | QKTQAISVLHEMTQQTFNLFSTKDSSATWEQSLLEKFSTELNQQLNDL |
| | | EACVIQGVGVEETPPMNVDSILAVKKYFQRITLYLTEKKYSPCAWEVV |
| | | RAEIMRSFSFSTNLQKRLRRKE |
| SEQ ID NO:58 | 1F6 | CDLPQTHSLGNRRTLMIMAQNGRISPFSCLKDRHDFGFPQEEFDGNQF |
| | | QKAQAISVLHEMIQQTFNLFSTKDSSATWEQSLLEKFSTELNQQLNDL |
| | | EACVIQEAGVEETPLNNVDSTLAVKKYFQRITLYLTEKKYSPCAWEW |
| | | RAEIMRSFSFSTNLQKRLRRKE |
| SEQ ID NO:59 | 2A10 | CDLPQTHSLGNRRALILLAQMGRISHFSCLKDRYDFGFPQEVFDGNQF |
| | | QKAQAISAFHEMIQQTFNLFSTKDSSATWEQSLLEKFSTELYQQLNNL |
| | | EACVIQEVGVEETPLMNEDSTLAVRKYFQRTTLYLMEKKYSPCAWEVV |
| | | RAETMRSFSFSTNLQKRLRRKE |
| SEQ ID NO:60 | 2C3 | CDLPQTHSLGNRRALILLAQMGRISPFSCLKDRHDFGFPQEEFDGNQS |
| | | QKAQAISVLHEMIQQTFNLFSTKDSSDTWDATLLEKFSTELNQQLNDL |
| | | EACVIQEVGVEETPLMNVDSILAVKKYFQRITLYLTEKKYSPCAWEVV |
| | | RAETMRSFSFSTNLQKRLRRKE |
| SEQ ID NO:61 | 2D1 | CDLPQTHSLGNRRALILLAQMRRISPFSCLKDRHDFGFPQEEFDGNQF |
| | | QKAQATSAFHEMIQQTFNLFSTKDSSAAWEQSLLEKFSTELYQQLNNL |
| | | EACVIQEVGMEETPLMNEDSILAVKKYFQRITLYLTEKKYSPCAWEVV |
| | | RAETMRSFSFSTNLQKRLRRKE |
| SEQ ID NO:62 | 2D10 | CDLPQTHSLGNRRALILLAQMGRVSPFSCLKDRHDFGFPQEEFDGNQF |
| | | QKAQAISAFHEMIQQTFNLFSTKDSSATWEQSLLEKFSTELYQQLNNL |
| | | EACVIQEVGVEETPLMNVDSILAVKKYFRRITLYLTEKKYSPCAWEVV |
| | | RAEIMRSFSFSTNLQKRLRRKE |
| SEQ ID NO:63 | 2D7 | CDLPQTHSLGNRRALILLAQMGRISPFSCLKDRHDFRFPQEEFDGNQF |
| | | QKAQAISVLHEMIQQTFNLFSTKDSSATWEQSLLEKFSTELYQQLNNL |
| | | EACVIQEVGVEETPLMNVDSILAVKKYFQRITLYLTERKYSPCAWEVV |
| | | RAEIMRSFSFSTNLQKRLRRKE |
| SEQ ID NO:64 | 2D9 | CDLPQTHSLGNRRALTLLAQMGRISPFSCLKDRHDFGFPQEEFDGNQF |
| | | QKAQAISVLHENIQQTFNLFSTKDSSATWEQSLLEKFSTELNQQLNDL |
| | | EACVIQEVGVEETPLVNVDSILAVKKYFQRITLYLTEKKYSPCAWEVV |
| | | RAEIMRSFSFSTNLQKRLRRKE |
| SEQ ID NO:65 | 2DA2 | CDLPQTHSLGNRRPLILLAQMGRISPFSCLKDRQDFGFPQEEFDGNQF |
| | | QKAQAISVLHEMMQQTFNLFSTKNSSAAWEQSLLEKFSTELHQQLNEL |
| | | EACVIQEVGVEETPLMNVDSILAVKKYFQRTTLYLIERKYSPCAWEVV |
| | | RAETMRSFSFSTNLQKRLRRKE |
| SEQ ID NO:66 | 2DH9 | CDLPQTHSPGNRRALMLLAQMGRISPFSCLKDRYDFGFPQGEFDGNQF |
| | | QKAQAISVLHENTAQQTFNLFSTKDSSAAWEQSLLEKFSTELYRQLNDL |
| | | EACVIQEVGVEETPLMNVDSILAVRKYFQRITLYLTEKKHSPCSWEVV |
| | | RAETMRSFSFSTNLQKRLRRKE |
| SEQ ID NO:67 | 2G11 | CDLPQTHSLGNRRALILLAQMGRISPFSCLKDRHDFGLPQEEFDGNQF |
| | | QKTQAISVLHEMIQQTFNLFSTKDSSDTWEQSLLEKFYIELFQQLNDL |
| | | EACVTQEVGVEETPLMNVDSILAVRKYFQRITLYLTEEKYSPCAWEVV |
| | | RAEIMRSFSFSTNLQKRLRRKE |
| SEQ ID NO:68 | 2G12 | CDLPQTHSLGNRRTLMLMAQMRRISPFPRLKDRYDFGFPQEVFDGNQF |
| | | QKAQATFLFHEMMQQTFNLFSTKNSSAAWDETLLDKFYTELYQQLNDL |
| | | EACVMQEGRVGETPLNNADSILAVKKYFRRITLYLTEKKYSPCAWEAV |
| | | RAEIMRSFSFSTNLQKRLRRKE |
| SEQ ID NO:69 | 2H9 | CDLPQTHSLGNRRALILLAQMGRISPFSCLKDRHDFGFPQEEFDGNQF |
| | | QKAQATSVLHEMIQQTFNLFSTKDSSATWEQSLLEKFSTELNQQLNDL |
| | | EACVTQEVGVEETPLNNEDSILAVKKYFQRITLYLTEKKYSPCAWEVV |
| | | RAEIMRSFSFSTNLQKRLRRKE |
| SEQ ID NO:70 | 6BC11 | CDLPQTHSLGNRRALILLAQNGRISPFSCLKDRYDFGFPQEEFDGNQL |
| | | QKAQAISVLHEMIQQTFNLFSTKDSSAAWEQSLLEKFSTELNQQLNDL |
| | | EACVIQEVGVEETPLHNVDSILAVKKYFQRITLYLTERKYSPCAWEVV |
| | | RAEIMRSFSFSTNLQKRLRRKE |
| SEQ ID NO:71 | 119bb | CDLPQTHSLGXXRAXXLLXQMXRXSXFSCLKDRXDFGXPXEEFDXXXF |
| | | QXXQAIXXXHEXXQQTFNXFSTKXSSXXWXXXLLXKXXTXLXQQLNXL |
| | | EACVXQXVXXXXTPLHNXDXTLAVXKYXQRITLYLXEXKYSPCXWEVV |
| | | RAETMRSFSFSTNLQKRLRRKE |
| SEQ ID NO:72 | CH1.1 | TGTGATCTGCCTCAGACCCACAGCCTTGGTAACAGGAGGGCCTTGATA |
| | | CTCCTGGCACAAATGGGAAGAATCTCTCCTTTCTCCTGTCTGATGGAC |
| | | AGACATGACTTTGGATTCCCCCAGGAGGAGTTTGATGGCAACCAGTTC |
| | | CAGAAGGCTCAAGCCATCTCTGTCCTCCATGAGATGATCCAACAGACC |
| | | TTCAATCTCTTCAGCACAAAGCACTCATCTGCTACTTGGGATGAGACA |
| | | CTTCTAGACAAATTCTACACTGAACTTTACCAGCAGCTGAATGACCTG |
| | | GAAGCCTGCGTGATACAGGAGGTTGGGGTGGAAGAGACTCCCCTGATG |
| | | AATGAGGACTCCATCTTGGCTGTGAAGAAATACTTCCGAAGAATCACT |
| | | CTCTATCTGACAGAGAAGAAATACAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:73 | CH 1.2 | TGTCATCTGCCTCAGACCCACAGCCTTGGTAACAGGAGGGCCTTGATA |
| | | CTCCTGGCACAAATGGGAAGAATCTCTCCTTTCTCCTGCCTGAAGGAC |
| | | AGACATGACTTTGGATTCCCCCAGGAGGAGTTTGATGGCAACCAGTTC |
| | | CAGAAGGCTCAAGGCATCTCTGTCCTCCATGAGATGATCCAGCAGACC |
| | | TTCCATCTCTTCAGCACAAAGGACTCATCTCCTACTTGGGAACAGAGC |
| | | CTCCTAGAAAAATTTTCCACTGAACTTAACCAGCAGCTGAATGACCTG |
| | | GAAGCCTGCGTGATACAGGAGGTTGGGGTGGAAGAGACTCCCCTGATG |
| | | AATGTGGACTCCATCCTGGCTGTGAAGAAATACTTCCGAAGAATCACT |
| | | CTTTATCTGACAGAGAAGAAATACAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:74 | CH1.3 | TGTGATCTGCCTCAGACCCACAGCCTTGGTAACAGGAGGACTTTGATG |
| | | ATAATGGCACAAATGGGAAGAATCTCTCCTTTCTCCTGCCTGAAGGAC |
| | | AGACATGAC TTTGGATTTCCTCAGGAGGAGTTTGATGGCAACCAGTTC |
| | | CAGAAGGCTCAACCCATCTCTGTCCTCCATGAGATGATCCAGCAGACC |
| | | TTCAATCTCTTCACCACAAAGGACTCATCTGCTACTTGGGATGAGACA |
| | | CTTCTAGACAAATTCTACACTGAACTTTACCAGCAGCTGAATGACCTG |
| | | GAAGCCTGTATGATGCAGGAGGTTGGAGTGGAAGACACTCCTCTGATG |
| | | AATGTGGACTCTATCCTGACTGTGAGAAAATACTTTCGAAGAATCACT |
| | | CTTTATCTGACAGACAAGAAATACAGCCCTTGTGCCTGCGAGGTTGTC |
| | | AGAGCAGAAATCATGAGATCTTTCTCTTTTTCAACAAACTTGCAAAAA |
| | | AGATTAAGGACGAAGGAA |
| SEQ ID NO:75 | CH1.4 | TGTGATCTGCCTCAGACCCACAGCCTGGGTAATACGAGGGCCTTGATA |
| | | CTCCTGGCACAAATGGGAAGAATCTCTCCTTTCTCCTGCCTGAAGGAC |
| | | AGACATGACTTTGCATTCCCCCAGGAGGAGTTTGGTGGCAACCAGTTC |
| | | CAGAAGGCTCAAGCCATCTCTGTCCTCCATGAGATGATCCAGCAGACC |
| | | TTCAATCTCTTCAGCACAGAGGACTCATCTGCTGCTTGGGATGAGACC |
| | | CTCCTAGACAAATTCTACATTGAACTTTTCCAGCAACTGAATGACCTG |
| | | GAAGCCTGTGTGATGCAGGAGGAGAGGGTGGGAGAAACTCCCCTGATG |
| | | AATGCGGACTCCATCTTGGCTGTGAAAGAAATACTTCCAAAGAATCACT |
| | | CTTTATCTGACAGAGAAGAAATACAGCCCTTGTGCCTGGGAGGTTGTC |
| | | ACAGCAGAAATCATGAGATCTTTCTCTTTTTCAACAAACTTCCAAAAA |
| | | AGATTAAGGAGGAACGAA |
| SEQ ID NO:76 | CH2.1 | TGTGATCTGCCTCAGACCCACAGCCTTCGTAACACGAGGACTTTGATG |
| | | ATAATGGCACAAATGGGAAGAATCTCTCCTTTCTCCTGCCTCAAGGAC |
| | | AGACATGACTTTGGATTTCCTCAGGAGGAGTTTGATGGCAACCAGTTC |
| | | CAGAAGCCTCAAGCCATCTCTGTCCTCCATGAGATGATCCAGCAGACC |
| | | TTCAATCTCTTCAGCACAAAGCACTCATCTGCTACTTGGGATGAGACA |
| | | CTTCTAGACAAATTCTACACTGAACTTTACCAGCAGCTGAATGACCTG |
| | | GAAGCCTGTATGATACAGGAGCTTGGGGTGGAAGAGACTCCCCTGATG |
| | | AATGAGGACTCCATCTTGGCTGTGAAGAAATACTTCCGAAGAATCACT |
| | | CTCTATCTGACAGAGAAGAAATACAGCCCTTGTGCCTGGGAGGTTGTC |
| | | ACAGCAGAAATCATGAGATCTTTCTCTTTTTCAACAAACTTGCAAAAA |
| | | AGATTAAGGAGGAAGGAA |
| SEQ ID NO:77 | CH2.2 | TGTGATCTGCCTCAGACCCACAGCCTTGGTAACAGGAGGGCCTTGATA |
| | | CTCCTGGCACAAATGGGAAGAATCTCTCCTTTCTCCTGTCTGATGGAC |
| | | AGACATGACTTTGGATTTCCCCAGGAGGAGTTTGATGACAACCAGTTC |
| | | CAGAAGGCTCAAGCCATCTCTGTCCTCCATGAGATGATCCAACAGACC |
| | | TTCAATCTCTTCAGCACAAAGGACTCATCTGCTACTTGGGATGAGACA |
| | | CTTCTAGACAAATTCTACACTGAACTTTACCAGCAGCTGAATGACCTG |
| | | GAAGCCTGTATGATGCAGGAGGTTGGAGTGGAAGACACTCCTCTGATG |
| | | AATGTGGACTCTATCCTGACTGTGAAGAAATACTTCCGAAGAATCACT |
| | | CTTTATCTGACAGAGAAGAAATACAGCCCTTGTGCCTGGGAGGTTGTC |
| | | AGAGCAGAAATCATGAGATCTTTCTCTTTTTCAACAAACTTGCAAAAA |
| | | AGATTAAGGAGGAAGGAA |
| SEQ ID NO:78 | CH2.3 | TGTGATCTGCCTCAGACCCACAGCCTTGGTAACAGGAGGACTTTGATG |
| | | ATAATGGCACAAATGGGAAGAATCTCTCCTTTCTCCTGCCTGAAGGAC |
| | | AGACATGACTTTGGATTTCCTCAGGAGGAGTTTGATGGCAACCAGTTC |
| | | CAGAAGGCTCAAGCCATCTCTGTCCTCCATGAGATCATCCAGCAGACC |
| | | TTCAATCTCTTCAGCACAAAGGACTCATCTGCTACTTGGGATGAGACA |
| | | CTTCTAGACAAATTCTACACTGAACTTTACCAGCAGCTGAATGACCTG |
| | | GAAGCCTGTATGATGCAGGAGGTTGGAGTGGAAGACACTCCTCTGATG |
| | | AATGAGGACTCCATCTTGGCTGTCAAGAATACTTCCGAAGAATCACT |
| | | CTCTATCTGACAGAGAAGAAATACAGCCCTTGTGCCTGGGAGGTTGTC |
| | | AGAGCAGAAATCATGAGATCTTTCTCTTTCTCAACAAACTTGCAAAAA |
| | | AGATTAAGGAGGAACGAA |
| SEQ ID NO:79 | CH1.1 | CDLPQTHSLGNRRALILLAQMGRISPFSCLMDRHDFGFPQEEFDDNQF |
| | | QKAQAISVLHEMIQQTFNLFSTKDSSATWDETLLDKFYTELYQQLNDL |
| | | EACVTQEVGVEETPLMNEDSILAVKKYFRRITLYLTEKKYSPCAWEVV |
| | | RAEIMRSFSFSTNLQKRLRRKE |
| SEQ ID NO:80 | CF1.2 | CDLPQTHSLGNRRALILLAQMGRISPFSCLKDRHDFGFPQEEFDGNQF |
| | | QKAQGISVLHEMIQQTFHLFSTKDSSATWEQSLLEKFSTELNQQLNDL |
| | | EACVIQEVGVEETPLMNVDSILAVKKYFRRTTLYLTEKKYSPCAWEVV |
| | | RAEIHRSFSFSTNLQKRLRRKE |
| SEQ ID NO:81 | CH1.3 | CDLPQTHSLGNRRTLMIMAQNCRISPFSCLKDRHDFGFPQEEFDGNQF |
| | | QKAQAISVLHEMTQQTFNLFSTKDSSATWDETLLDKFYTELYQQLNDL |
| | | EACMMQEVGVEDTPLMNVDSTLTVRKYFRRITLYLTEKKYSPCAWEVV |
| | | RAETMRSFSFSTNLQKRLRRKE |
| SEQ ID NO:82 | CH1.4 | CDLPQTHSLGNRRALTLLAQMGRTSPFSCLKDRHDFGFPQEEFGGNQF |
| | | QKAQAISVLHEMIQQTFNLFSTEDSSAAWDETLLDKFYIELFQQLNDL |
| | | EACVMQEERVGETPLMNADSILAVKKYFQRITLYLTEKKYSPCAWEVV |
| | | RAETMRSFSFSTNLQKRLRRKE |
| SEQ ID NO:83 | CH2.1 | CDLPQTHSLGNRRTLMIMAQMGRISPFSCLKDRHDFGFPQEEFDGNQF |
| | | QKAQAISVLHEMIQQTFNLFSTKDSSATWDETLLDKFYTELYQQLNDL |
| | | EACMTQEVGVEETPLMNEDSILAVKKYFRRTTLYLTEKKYSPCAWEVV |
| | | RAEIMRSFSFSTNLQKRLRRKE |
| SEQ ID NO:84 | CH2.2 | CDLPQTHSLGNRPALILLAQMGRISPFSCLNDRHDFGFPQEEFDDNQF |
| | | QKAQAISVLHEMTQQTFNLFSTKDSSATWDETLLDKFYTELYQQLNDL |
| | | EACHNMQEVGVEETPLMINDSILTVKKYFRRITLYLTEKKYSPCAWEW |
| | | RAEIMRSFSFSTNLQKRLRRKE |
| SEQ ID NO:85 | CH2.3 | CDLPQTHSLGNRRTLMIMAQMGRISPFSCLKDRHDFGFPQEEFDGNQF |
| | | QKAQAISVLHEMTQQTFNLFSTKDSSATWDETLLDKFYTELYQQLNDL |
| | | EACMMQEVGVEETPLMNEDSILAVKKYFRRITLYLTEKKYSPCAWEVV |
| | | RAEIMRSFSFSTNLQKRLRRKE |

What is claimed is:

1. An isolated or recombinant nucleic acid, comprising:
a polynucleotide sequence selected from the group consisting of:
(a) SEQ ID NO:1 to SEQ ID NO:35, or a complementary polynucleotide sequence thereof;
(b) a polynucleotide sequence encoding a polypeptide selected from SEQ ID NO:36 to SEQ ID NO:70, or a complementary polynucleotide sequence thereof;
(c) a polynucleotide sequence which hybridizes under highly stringent conditions over substantially the entire length of polynucleotide sequence (a) or (b); and
(d) a polynucleotide sequence comprising a fragment of (a), (b), or (c), which fragment encodes a polypeptide having antiproliferative activity in a human Daudi cell line-based assay.

2. An isolated or recombinant nucleic acid, comprising:
a polynucleotide sequence selected from the group consisting of:
(a) SEQ ID NO:72 to SEQ ID NO:78, or a complementary polynucleotide sequence thereof;
(b) a polynucleotide sequence encoding a polypeptide selected from SEQ ID NO:79 to SEQ ID NO:85, or a complementary polynucleotide sequence thereof;
(c) a polynucleotide sequence which hybridizes under highly stringent conditions over substantially the entire length of polynucleotide sequence (a) or (b); and
(d) a polynucleotide sequence comprising a fragment of (a), (b) or (c), which fragment encodes a polypeptide having antiviral activity in a murine cell line/EMCV-based assay.

3. An isolated or recombinant nucleic acid, comprising:
a polynucleotide sequence encoding a polypeptide, the polypeptide comprising the amino acid sequence: CDLPQTHSLG-X₁₁-X₁₂-RA-X₁₅-X₁₆-LL-X₁₉-QM-X₂₂-R-X₂₄-S-X₂₆-FSCLKDR-X₃₄-DFG-X₃₈-P-X₄₀-EEFD-X₄₅-X₄₆-X₄₇-FQ-X₅₀-X₅₁-QAI-X₅₅-X₅₆-X₅₇-HE-X₆₀-X₆₁-QQTFN-X₆₇-FSTK-X₇₂-SS-X₇₅-X₇₆-W-X₇₈-X₇₉-X₈₀-LL-X₈₃-K-X₈₅-X₈₆-T-X₈₈-L-X₉₀-QQLN-X₉₅-LEACV-X₁₀₁-Q-X₁₀₃-V-X₁₀₅-X₁₀₆-X₁₀₇-X₁₀₈-TPLMN-X₁₁₄-D-X116-ILAV-X₁₂₁-KY-X₁₂₄-QRITLYL-X₁₃₂-E-X₁₃₄-KYSPC-X₁₄₀-WEVVRAEIMRSFSFSTNLQKRLRRKE, or a conservatively substituted variation thereof, where X₁₁ is N or D; X₁₂ is R, S, or K; X₁₅ is L or M; X₁₆ is I, M, or V; X₁₉ is A or G; X₂₂ is G or R; X₂₄ is I or T; X₂₆ is P or H; X₃₄ is H, Y or Q; X₃₈ is F or L; X₄₀ is Q or R; X₄₅ is G or S; X₄₆ is N or H; X₄₇ is Q or R; X₅₀ is K or R; X₅₁ is A or T; X₅₅ is S or F; X₅₆ is V or A; X₅₇ is L or F; X₆₀ is M or I; X₆₁ is I or M; X₆₇ is L or F; X₇₂ is D or N; X₇₅ is A or V; X₇₆ is A or T; X₇₈ is E or D; X₇₉ is Q or E; X₈₀ is S, R, T, or N; X₈₃ is E or D; X₈₅ is F or L; X₈₆ is S or Y; X₈₈ is E or G; X₉₀ is Y, H, N; X₉₅ is D, E, or N; X₁₀₁ is I, M, or V; X₁₀₃ is E or G; X₁₀₅ is G or W; X₁₀₆ is V or M; X₁₀₇ is E, G, or K; X₁₀₈ is E or G; X₁₁₄ is V, E, or G; X₁₁₆ is S or P; X₁₂₁ is K or R; X₁₂₄ is F or L; X₁₃₂ is T, I, or M; X₁₃₄ is K or R; and X₁₄₀is A or S.

4. The nucleic acid of claim 3, said polypeptide having antiproliferative activity in a human Daudi cell line-based cell proliferation assay or antiviral activity in a human WISH cell/EMCV-based assay.

5. The nucleic acid of claim 3, wherein the encoded polypeptide has an antiproliferative activity of at least about 8.3×10⁶ units/milligram in a human Daudi cell line-based assay or an antiviral activity of at least about 2.1×10⁷ units/milligram in a human WISH cell/EMCV-based assay.

6. The nucleic acid of claim 3, wherein the encoded polypeptide comprises an amino acid sequence selected from the group consisting of: SEQ ID NO:36 to SEQ ID NO:54.

7. The nucleic acid of claim 3, said nucleic acid comprising a polynucleotide sequence selected from the group consisting of: SEQ ID NO:1 to SEQ ID NO:19.

8. An isolated or recombinant nucleic acid comprising a polynucleotide sequence encoding a polypeptide, the polypeptide comprising:
an amino acid sequence comprising at least 20 contiguous amino acids of any one of SEQ ID NOS:36-70, and one or more of amino acids Ala19, (Tyr or Gln)34, Gly37, Phe38, Lys71, Ala76, Tyr90, Ile132, Arg134, Phe152, Lys160, and Glu166, wherein the numbering of the amino acids corresponds to that of SEQ ID NO:36.

9. The nucleic acid of claim 8, wherein the encoded polypeptide is 166 amino acids in length.

10. The nucleic acid of claim 8, wherein the encoded polypeptide has an antiproliferative activity in a human Daudi cell line-based assay.

11. The nucleic acid of claim 8, wherein the encoded polypeptide has an antiviral activity in a human WISH cell/EMCV-based assay.

12. The nucleic acid of claim 8, wherein the encoded polypeptide comprises amino acids Ala19, (Tyr or Gln)34, Gly37, Phe38, Lys71, Ala76, Tyr90, Ile132, Arg134, Phe152, Lys160, and Glu166.

13. The nucleic acid of claim 8, wherein the encoded polypeptide comprises at least 50 contiguous amino acid residues of any one of SEQ ID NOS:36-70.

14. The nucleic acid of claim 8, wherein the encoded polypeptide comprises at least 100 contiguous amino acid residues of any one of SEQ ID NOS:36-70.

15. The nucleic acid of claim 8, wherein the encoded polypeptide comprises at least 150 contiguous amino acid residues of any one of SEQ ID NOS:36-70.

16. The nucleic acid of claim 8, wherein the encoded polypeptide comprises an amino acid sequence selected from the group consisting of: SEQ ID NO:36, SEQ ID NO:37, SEQ ID NO:39, SEQ ID NO:40, SEQ ID NO:41, SEQ ID NO:42, SEQ ID NO:45, and SEQ ID NO:46.

17. The nucleic acid of claim 8, comprising a polynucleotide sequence selected from the group consisting of: SEQ ID NO:1, SEQ ID NO:2, SEQ ID NO:4, SEQ ID NO:5, SEQ ID NO:6, SEQ ID NO:7, SEQ ID NO:10, and SEQ ID NO:11.

18. An isolated or recombinant nucleic acid comprising a polynucleotide sequence encoding a polypeptide, the polypeptide comprising:
an amino acid sequence comprising at least 155 contiguous amino acids of any one of SEQ ID NOS:36-70, said amino acid sequence comprising amino acids Lys160 and Glu166, wherein the numbering of the amino acids corresponds to that of SEQ ID NO:36.

19. The nucleic acid of claim 18, wherein the encoded polypeptide comprises an amino acid sequence selected from the group consisting of: SEQ ID NO:36, SEQ ID NO:37, SEQ ID NO:39, SEQ ID NO:40, SEQ ID NO:41, SEQ ID NO:42, SEQ ID NO:45, and SEQ ID NO:46.

20. A cell comprising the nucleic acid of claim 1, 2, 8, or 18.

21. The cell of claim 20, wherein the cell expresses a polypeptide encoded by the nucleic acid.

22. A vector comprising the nucleic acid of claim 1, 2, 8, or 18.

23. The vector of claim 20, wherein the vector comprises a plasmid, a cosmid, a phage, or a virus.

24. The vector of claim 22, wherein the vector is an expression vector.

25. A cell transduced by the vector of claim 22.

26. A composition comprising the nucleic acid of claim 1, 2, 8, or 18, and an excipient.

27. The composition of claim 26, wherein the excipient is a pharmaceutically acceptable excipient.

28. A composition produced by digesting one or more nucleic acids of claim 1, 2, 3, 8, or 18 with a restriction endonuclease, an RNAse, or a DNAse.

29. A composition produced by a process comprising incubating one or more nucleic acids of claim 1, 2, 3, 8, or 18 in the presence of deoxyribonucelotide triphosphates and a nucleic acid polymerase.

30. The composition of claim 29, wherein the nucleic acid polymerase is a thermostable polymerase.

31. An isolated or recombinant polypeptide encoded by the nucleic acid of acid claim 1, 2, 3, 8, or 18.

32. The isolated or recombinant polypeptide of claim 31, comprising a sequence selected from the group consisting of: SEQ ID NO:36 to SEQ ID NO:70 or SEQ ID NO:79 to SEQ ID NO:85.

33. The polypeptide of claim 31, having an antiproliferative activity of at least about 8.3×10⁶ units/milligram (mg) in a human Daudi cell line-based assay or an antiviral activity of at least about 2.1×10⁷ units/milligram in a human WISH cell/EMCV-based assay.

34. An isolated or recombinant polypeptide, comprising:
the amino acid sequence: CDLPQTHSLG-X₁₁-X₁₂-RA-X₁₅-X₁₆-LL-X₁₉-QM-X₂₂-R-X₂₄-S-X₂₆-FSCLKDR-X₃₄-DFG-X₃₈-P-X₄₀-EEFD-X₄₅-X₄₆-X₄₇-FQ-X₅₀-X₅₁-QAI-X₅₅-X₅₆-X₅₇-HE-X₆₀-X₆₁-QQTFN-X₆₇-FSTK-X₇₂-SS -X₇₅-X₇₆-W-X₇₈-X₇₉-X₈₀-LL-X₈₃-K-X₈₅-X₈₆-T-X₈₈-L-X₉₀-QQLN-X₉₅-LEACV-X₁₀₁-Q-X₁₀₃-V-X₁₀₅-X₁₀₆-X₁₀₇-X₁₀₈-TPLMN-X₁₁₄-D-X₁₁₆-ILAV-X₁₂₁-KY-X₁₂₄-QRITLYL-X₁₃₂-E-X₁₃₄-KYSPC-X₁₄₀-WEVVRAEIMRSFSFSTNLQKRLRRKE, or a conservatively substituted variation thereof;
wherein X₁₁ is N or D; X₁₂ is R, S, or K; X₁₅ is L or M; X₁₆ is I, M, or V; X₁₉ is A or G; X₂₂ is G or R; X₂₄ is I or T; X₂₆ is P or H; X₃₄ is H, Y or Q; X₃₈ is F or L; X₄₀ is Q or R; X₄₅ is G or S; X₄₆ is N or H; X₄₇ is Q or R; X₅₀ is K or R; X₅₁ is A or T; X₅₅ is S or F; X₅₆ is V or A; X₅₇ is L or F; X₆₀ is M or I; X₆₁ is I or M; X₆₇ is L or F; X₇₂ is D or N; X₇₅ is A or V; X₇₆ is A or T; X₇₈ is E or D; X₇₉ is Q or E; X₈₀ is S, R, T, or N; X₈₃ is E or D; X₈₅ is F or L; X₈₆ is S or Y; X₈₈ is E or G; X₉₀ is Y, H, N; X₉₅ is D, E, or N; X₁₀₁ is I, M, or V; X₁₀₃ is E or G; X₁₀₅ is G or W; X₁₀₆ is V or M; X₁₀₇ is E, G, or K; X₁₀₈ is E or G; X₁₁₄ is V, E, or G; X₁₁₆ is S or P; X₁₂₁ is K or R; X₁₂₄ is F or L; X₁₃₂ is T, I, or M; X₁₃₄ is K or R; and X₁₄₀ is A or S.

35. The polypeptide of claim 34, having antiproliferative activity of at least about 8.3×10⁶ units/milligram in a human Daudi cell line-based assay or antiviral activity of at least about 2.1×10⁷ units/milligram in a human WISH cell/EMCV-based assay.

36. The polypeptide of claim 34, comprising a sequence selected from the group consisting of: SEQ ID NO:36 to SEQ ID NO:54.

37. A polypeptide comprising at least 100 contiguous amino acids of a protein encoded by a coding polynucleotide sequence, the polynucleotide sequence selected from the group consisting of:
(a) SEQ ID NO:1 to SEQ ID NO:35 or SEQ ID NO:72 to SEQ ID NO:78;
(b) a coding polynucleotide sequence that encodes a first polypeptide selected from SEQ ID NO:36 to SEQ ID NO:70 or SEQ ID NO:79 to SEQ ID NO:85; and
(c) a complementary polynucleotide sequence which hybridizes under highly stringent conditions over substantially an entire length of a polynucleotide sequence of (a) or (b).

38. The polypeptide of claim 37, said polypeptide having an antiproliferative activity in a human Daudi cell line-based cell proliferation assay or an antiviral activity in a human WISH cell/EMCV-based assay.

39. The polypeptide of claim 37, wherein the polypeptide specifically binds to a human alpha-interferon receptor.

40. The polypeptide of claim 37, comprising at least 150 contiguous amino acids of the encoded protein.

41. An isolated or recombinant polypeptide, comprising: an amino acid sequence comprising at least 50 contiguous amino acids of any one of SEQ ID NOS:36-70, the amino acid sequence comprising one or more of amino acids Ala19, (Tyr or Gln)34, Gly37, Phe38, Lys71, Ala76, Tyr90, Ile132, Arg134, Phe152, Lys160, and Glu166, wherein the numbering of the amino acids corresponds to that of SEQ ID NO:36.

42. The polypeptide of claim 41, wherein the polypeptide binds a human alpha-interferon receptor.

43. The polypeptide of claim 41, said polypeptide exhibiting an antiproliferative activity in a human Daudi cell line-based cell proliferation assay or an antiviral activity in a human WISH cell/EMCV-based assay.

44. The polypeptide of claim 41, having an antiproliferative activity of at least about 8.3×10⁶ units/milligram in a human Daudi cell line-based assay or an antiviral activity of at least about 2.1×10⁷ units/milligram in a human WISH cell/EMCV-based assay.

45. The polypeptide of claim 41, wherein the polypeptide is 166 amino acids in length.

46. The polypeptide of claim 41, said polypeptide comprising amino acids Ala19, (Tyr or Gln)34, Gly37, Phe38, Lys71, Ala76, Tyr90, Ile132, Arg134, Phe152, Lys160, and Glu166, wherein the numbering of the amino acids of said polypeptide corresponds to the numbering of amino acids in SEQ ID NO:36.

47. The polypeptide of claim 41, comprising at least 100 contiguous amino acid residues of any one of SEQ ID NOS:36-70.

48. The polypeptide of claim 41, comprising at least 150 contiguous amino acid residues of any one of SEQ ID NOS:36-70.

49. The polypeptide of claim 41, comprising at least 155 contiguous amino acid residues of any one of SEQ ID NOS:36-70.

50. The polypeptide of claim 41, comprising an amino acid sequence selected from the group consisting of: SEQ ID NO:36, SEQ ID NO:37, SEQ ID NO:39, SEQ ID NO:40, SEQ ID NO:41, SEQ ID NO:42, SEQ ID NO:45, and SEQ ID NO:46.

51. An isolated or recombinant polypeptide comprising an amino acid sequence comprising at least 155 contiguous amino acids of any one of SEQ ID NOS:36-70, the isolated or recombinant polypeptide comprising amino acids Lys160 and Glu166, wherein the numbering of the amino acids corresponds to that of SEQ ID NO:36.

52. The polypeptide of claim 51, comprising an amino acid sequence selected from the group consisting of: SEQ ID NO:36, SEQ ID NO:37, SEQ ID NO:39, SEQ ID NO:40, SEQ ID NO:41, SEQ ID NO:42, SEQ ID NO:45, and SEQ ID NO:46.

53. The polypeptide of claim 51, said polypeptide having an antiproliferative activity of at least about 8.3×10⁶ units/milligram in milligram in a human Daudi cell line-based assay or an antiviral activity of at least about 2.1×10⁷ units/milligram in a human WISH cell/EMCV-based assay.

54. The polypeptide of claim 31, 34, 37, 41, or 51, further comprising a secretion/localization sequence.

55. The polypeptide of claim 31, 34, 37, 41, or 51, further comprising a polypeptide purification subsequence.

56. The polypeptide of claim 55, wherein the sequence that facilitates purification is selected from the group consisting of: an epitope tag, a FLAG tag, a polyhistidine tag, and a GST fusion.

57. The polypeptide of claim 31, 34, 37, 41, or 51, further comprising a Met at the N-terminus.

58. The polypeptide of claim 31, 34, 37, 41, or 51, comprising a modified amino acid.

59. The polypeptide of claim 58, wherein the modified amino acid is selected from the group consisting of: a glycosylated amino acid, a PEGylated amino acid, a farnesylated amino acid, an acetylated amino acid, and a biotinylated amino acid.

60. A composition comprising the polypeptide of claim 31, 34, 37, 41, or 51 and an excipient.

61. The composition of claim 60, wherein the excipient is a pharmaceutically acceptable excipient.

62. A composition comprising the polypeptide of claim 58 in a pharmaceutically acceptable excipient.

63. A polypeptide which is specifically bound by a polyclonal antisera raised against at least one antigen, said at least one antigen comprising at least one amino acid sequence of SEQ ID NO:36 to SEQ ID NO:70 or SEQ ID NO:79 to SEQ ID NO:85, or a fragment thereof, wherein the antisera is subtracted with an IFN-alpha polypeptide encoded by a nucleic acid corresponding to one or more of GenBank accession number: J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1).

64. An antibody or antisera produced by administering the polypeptide of claim 31, 34, 37, 41, or 51 to a mammal, which antibody or antisera specifically binds at least one antigen, said at least one antigen comprising a polypeptide comprising one or more of the amino acid sequences of SEQ ID NO:36 to SEQ ID NO:70 and SEQ ID NO:79 to SEQ ID NO:85, or a fragment thereof, which antibody or antisera does not specifically bind to an IFN-α polypeptide encoded by a nucleic acid corresponding to one or more of GenBank accession number: J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1).

65. An antibody or antisera which specifically binds a polypeptide, the polypeptide comprising a sequence selected from the group consisting of: SEQ ID NO:36 to SEQ ID NO:70 or SEQ ID NO:79 to SEQ ID NO:85, wherein the antibody or antisera does not specifically bind to an IFN-alpha polypeptide encoded by a nucleic acid corresponding to one or more of GenBank accession number: J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1).

66. A method of producing a polypeptide, the method comprising:
introducing into a population of cells a nucleic acid of claim 1, 2, 3, 8, or 18, the nucleic acid operatively linked to a regulatory sequence effective to produce the encoded polypeptide; and
culturing the cells in a culture medium to produce the polypeptide.

67. A method of producing a polypeptide, the method comprising:
introducing into a population of cells a recombinant expression vector comprising the nucleic acid of claim 1, 2, 3, 8, or 18; and
culturing the cells in a culture medium under conditions suitable to produce the polypeptide encoded by the expression vector.

68. A method of inhibiting growth of population of tumor cells, the method comprising:
contacting the population of tumor cells with an effective amount of a polypeptide of claim 31, 34, 37, 41, or 51 sufficient to inhibit growth of tumor cells in said population of tumor cells, thereby inhibiting growth of tumor cells in said population of cells.

69. The method of claim 68, wherein the tumor cells are selected from the group consisting of: human carcinoma cells, human leukemia cells, human T-lymphoma cells, and human melanoma cells.

70. The method of claim 68, wherein the tumor cells are in culture.

71. A method of inhibiting the replication of a virus within at least one cell infected by the virus, the method comprising:
contacting said at least one infected cell with an effective amount of a polypeptide of claim 31, 34, 37, 41, or 51 sufficient to inhibit viral replication in said at least one infected cell, thereby inhibiting replication of the virus in said at least one infected cells.

72. The method of claim 71, wherein the virus is an RNA virus.

73. The method of claim 72, wherein the virus is a human immunodeficiency virus or a hepatitis C virus.

74. The method of claim 71, wherein the virus is a DNA virus.

75. The method of claim 74, wherein the virus is a hepatitis B virus.

76. The method of claim 71, wherein the cells are cultured.

77. A method of treating an autoimmune disorder in a patient, the method comprising: administering to the patient an effective amount of the polypeptide of claim 31, 34, 37, 41, or 51.

78. The method of claim 77, wherein the autoimmune disorder is selected from the group consisting of multiple sclerosis, rheumatoid arthritis, lupus erythematosus, and type I diabetes.

79. In a method of treating a disorder treatable by administration of interferon-alpha to a subject, an improved method comprising: administering to the subject an effective amount of the polypeptide of claim 31, 34, 37, 41, or 51.

80. The method claim 79, wherein the disorder treatable by administration of interferon-alpha is selected from the group consisting of: sclerosis, rheumatoid arthritis, lupus erythematosus, and type I diabetes.

81. A method of for making a modified or recombinant nucleic acid, the method comprising:
recursively recombining a sequence of one or more nucleic acids of claim 1, 2, 3, 8, or 18 with a sequence of one or more additional nucleic acids, each sequence of the one or more additional nucleic acids encoding an interferon-alpha or an amino acid subsequence thereof.

82. The method of claim 81, wherein said recursive recombination produces at least one library of recombinant interferon-alpha homologue nucleic acids.

83. A nucleic acid library produced by the method of claim 82.

84. A population of cells comprising the library of claim 83.

85. A recombinant interferon-alpha homologue nucleic acid produced by the method of claim 82.

86. A cell comprising the nucleic acid of claim 85.

87. The method of claim 81, wherein the recursive recombination is performed in vitro.

88. The method of claim 81, wherein the recursive recombination is performed in vivo or ex vivo.

89. A composition comprising two or more nucleic acids of claim 1, 2, 3, 8, or 18.

90. The composition of claim 89, wherein the composition comprises a library comprising at least ten nucleic acids.

91. A method of producing a modified or recombinant interferon-alpha homologue nucleic acid comprising mutating a nucleic acid of claim 1, 2, 3, 8, or 18.

92. The modified or recombinant interferon-alpha homologue nucleic acid produced by the method of claim 91.

93. A computer or computer readable medium comprising a database comprising a sequence record comprising one or more character strings corresponding to a nucleic acid or protein sequence selected from SEQ ID NO:1 to SEQ ID NO:85.

94. An integrated system comprising a computer or computer readable medium comprising a database comprising one or more sequence records, each of said sequence records comprising one or more character strings corresponding to a nucleic acid or protein sequence selected from SEQ ID NO:1 to SEQ ID NO:85, the integrated system further comprising a user input interface allowing a user to selectively view said one or more sequence records.

95. The integrated system of claim 94, the computer or computer readable medium comprising an alignment instruction set which aligns the character strings with one or more additional character strings corresponding to a nucleic acid or protein sequence.

96. The integrated system of claim 95, wherein the instruction set comprises one or more of: a local homology comparison determination, a homology alignment determination, a search for similarity determination, and a BLAST determination.

97. The integrated system of claim 95, further comprising a user readable output element which displays an alignment produced by the alignment instruction set.

98. The integrated system of claim 94, the computer or computer readable medium further comprising an instruction set which translates at least one nucleic acid sequence comprising a sequence selected from SEQ ID NO:1 to SEQ ID NO:35 or SEQ ID NO:72 to SEQ ID NO:78 into an amino acid sequence.

99. The integrated system of claim 94, the computer or computer readable medium further comprising an instruction set for reverse-translating at least one amino acid sequence comprising a sequence selected from SEQ ID NO:36 to SEQ ID NO:70 or SEQ ID NO:79 to SEQ ID NO:85 into a nucleic acid sequence.

100. The integrated system of claim 99, wherein the instruction set selects the nucleic acid sequence by applying a codon usage instruction set or an instruction set which determines sequence identity to a test nucleic acid sequence.

101. A method of using a computer system to present information pertaining to at least one of a plurality of sequence records stored in a database, said sequence records each comprising at least one character string corresponding to SEQ ID NO:1 to SEQ ID NO:85, the method comprising:
determining a list of at least one character string corresponding to one or more of SEQ ID NO:1 to SEQ ID NO:85 or a subsequence thereof;
determining which of said at least one character string of said list are selected by a user; and
displaying each of the selected character strings, or aligning each of the selected character strings with an additional character string.

102. The method of claim 101, further comprising displaying an alignment of each of the selected character strings with the additional character string.

103. The method of claim 101, further comprising displaying the list.

104. A nucleic acid which comprises a unique subsequence in a nucleic acid selected from SEQ ID NO:1 to SEQ ID NO:35 or SEQ ID NO:72 to SEQ ID NO:78, wherein the unique subsequence is unique as compared to a nucleic acid sequence of a known interferon-alpha nucleic acid sequence or a nucleic acid corresponding to any of GenBank accession number: J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1).

105. A polypeptide which comprises a unique subsequence in a polypeptide selected from: SEQ ID NO:36 to SEQ ID NO:70 or SEQ ID NO:79 to SEQ ID NO:85, wherein the unique subsequence is unique as compared to a sequence of a known interferon-alpha polypeptide or a sequence of a polypeptide encoded by a nucleic acid corresponding to any of GenBank accession number: J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1).

106. A target nucleic acid which hybridizes under stringent conditions to a unique coding oligonucleotide which encodes a unique subsequence in a polypeptide selected from: SEQ ID NO:36 to SEQ ID NO:70 or SEQ ID NO:79 to SEQ ID NO:85, wherein the unique subsequence is unique as compared to a sequence of a known interferon-alpha polypeptide or a sequence of a polypeptide encoded by a nucleic acid corresponding to any of GenBank accession number: J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1).

107. The nucleic acid of claim 106, wherein the stringent conditions are selected such that a perfectly complementary oligonucleotide to the unique coding oligonucleotide hybridizes to the unique coding oligonucleotide with at least a 5× higher signal to noise ratio than for hybridization of the perfectly complementary oligonucleotide to a control nucleic acid corresponding to any of GenBank accession number: J00210 (alpha-D), J00207 (Alpha-A), X02958 (Alpha-6), X02956 (Alpha-5), V00533 (alpha-H), V00542 (alpha-14), V00545 (IFN-1B), X03125 (alpha-8), X02957 (alpha-16), V00540 (alpha-21), X02955 (alpha-4b), V00532 (alpha-C), X02960 (alpha-7), X02961 (alpha-10 pseudogene), R0067 (Gx-1), I01614, I01787, I07821, M12350 (alpha-F), M38289, V00549 (alpha-2a), and I08313 (alpha-Con1), wherein the target nucleic acid hybridizes to the unique coding oligonucleotide with at least a 2× higher signal to noise ratio as compared to hybridization of the control nucleic acid to the coding oligonucleotide.

108. The nucleic acid of any of claims 1, 2, 3, 8, or 18, wherein the nucleic acid encodes an interferon-alpha homologue having an increased growth inhibition activity against a population of cancer cells relative to a growth inhibition activity of human interferon-alpha 2a against said population of cancer cells.

109. The nucleic acid of claim 108, wherein the cancer cells of said population of cancer cells comprise a cancer cell line selected from: a leukemia cell line, a melanoma cell line, a lung cancer cell line, a colon cancer cell line, a central nervous system (CNS) cancer cell line, an ovarian cancer cell line, a breast cancer cell line, a prostate cancer cell line, and a renal cancer cell line, and the growth inhibition activity is measured as a concentration of interferon-alpha homologue producing a 50% inhibition of growth of the cancer cell line (GI50 value), wherein the interferon-alpha homologue has a GI50 value at least 2-fold lower than the GI50 value of the human interferon-alpha 2a.

110. The nucleic acid of claim 109, wherein the encoded interferon-alpha homologue has a GI50 value at least 5-fold lower than the GI50 value of the human interferon-alpha 2a.

111. The nucleic acid of claim 107, wherein the encoded interferon-alpha homologue has a GI50 value at least 10-fold lower than the GI50 value of the human interferon-alpha 2a.

112. The nucleic acid of any of claims 1, 2, 3, 8, or 18, wherein the nucleic acid encodes an interferon-alpha homologue having increased an cytostatic activity against a population of cancer cells relative to the cytostatic activity of human interferon-alpha 2a against said population of cancer cells.

113. The nucleic acid of claim 112, wherein the cancer cells comprise a cancer cell line selected from: a leukemia cell line, a melanoma cell line, a lung cancer cell line, a colon cancer cell line, a CNS cancer cell line, an ovarian cancer cell line, a breast cancer cell line, a prostate cancer cell line, and a renal cancer cell line, the cytostatic activity measured as the concentration of an interferon-alpha causing a total inhibition of growth of the cell line (TGI value), wherein the interferon-alpha homologue has a TGI value at least 2-fold lower than the TGI value of the human interferon-alpha 2a.

114. The nucleic acid of claim 112, wherein the encoded interferon-alpha homologue has a TGI value at least 5-fold lower than the TGI value of the human interferon-alpha 2a.

115. The nucleic acid of claim 112, wherein the encoded interferon-alpha homologue has a TGI value at least 10-fold lower than the TGI value of the human interferon-alpha 2a.

116. The nucleic acid of any of claims 1, 2, 3, 8, or 18, wherein the nucleic acid encodes an interferon-alpha homologue having an increased cytotoxic activity against a population of cancer cells relative to the cytotoxic activity of human interferon-alpha 2a against said population of cancer cells.

117. The nucleic acid of claim 116, wherein the cancer cells comprise a cancer cell line selected from: a leukemia cell line, a melanoma cell line, a lung cancer cell line, a colon cancer cell line, a central nervous system (CNS) cancer cell line, an ovarian cancer cell line, a breast cancer cell line, a prostate cancer cell line, and a renal cancer cell line, the cytotoxic activity measured as the concentration of interferon-alpha producing a 50% reduction in an amount of cellular protein in a cell line measured after a period of incubation (LC50 value), wherein the interferon-alpha homologue has a LC50 value at least 2-fold lower than the LC50 value of the human interferon-alpha 2a.

118. The nucleic acid of claim 116, wherein the encoded interferon-alpha homologue has a LC50 value at least 5-fold lower than the LC50 value of the human interferon-alpha 2a.

119. The nucleic acid of claim 116, wherein the encoded interferon-alpha homologue has a LC50 value at least 10-fold lower than the LC50 value of the human interferon-alpha 2a.

120. The polypeptide of any of claims claim 31, 34, 37, 41, or 51, said polypeptide having an increased growth inhibition activity against a population of cancer cells relative to the inhibition activity of human interferon-alpha 2a against the population of cancer cells.

121. The polypeptide of claim 120, wherein the population of cancer cells comprises a cancer cell line selected from: a leukemia cell line, a melanoma cell line, a lung cancer cell line, a colon cancer cell line, a CNS cancer cell line, an ovarian cancer cell line, a breast cancer cell line, a prostate cancer cell line, and a renal cancer cell line, the growth inhibition activity measured as the concentration of polypeptide or human interferon-alpha 2a causing a 50% inhibition of growth of the cell line (GI50 value), wherein the polypeptide has a GI50 value at least 2-fold lower than the GI50 value of the human interferon-alpha 2a.

122. A nucleic acid produced by the method of claim 81.

123. An interferon-alpha polypeptide or amino acid subsequence thereof produced by the method of claim 81.


SEQUENCE LISTING (88 sequences)

SEQ ID NO: 1 - DNA, 498 bp, Artificial Sequence
  Description of Artificial Sequence Synthetic DNA
tgtgatctgcctcagacccacagccttggcaacaggagggccttgatgctcctggcacaaatgggacgaatctctcctttctcctgcctgaaggacagacaagactttggattcccccaggaggagtttgatggcaaccagttccagaaggctcaagccatctctgtcctccatgagatgatccagcagaccttcaatctcttcagcacaaaggattcatctgctgcttgggaacagaccctcctagaaaaattttccactgaactctaccagcagctgaatgacctggaagcctgcgtgatacaggaggtaggggtgaaagagactcccctgatgaatgtggactccatcctggctgtgaggaagtacttccaaagaatcactctttatctaatagagaggaaatacagcccttgtgcatgggaggttgtcagagcagaaatcatgagatctttctctttttcaacaaacttgcaaaaaagattaaggaggaaggaa

SEQ ID NO: 2 - DNA, 498 bp, Artificial Sequence
  Description of Artificial Sequence Synthetic DNA
tgtgatctgcctcagacccacagccttggtgacaggagggccatgatactcctggcacaaatgggacgaatctctcctttctcctgcctgaaggacagatatgatttcggattcccccaggaggagtttgatggcaaccagttccagaaggctcaagccatctctgtcctccatgagatgatccagcagaccttcaatctcttcagcacaaaggattcatctgctgcttgggaacagagcctcctagaaaaattttccactgaactttaccagcagctgaatgaactggaagcatgtgtgatacaggaggttggggtgggagagactcccctgatgaatggggactccatcctggctgtgaagaagtacttccaaagaatcactctttatctaatagagaggaaatacagcccttgtgcatgggaggttgtcagagcagaaatcatgagatctttctctttttcaacaaacttgcaaaaaagattaaggaggaaggaa

SEQ ID NO: 3 - DNA, 498 bp, Artificial Sequence
  Description of Artificial Sequence Synthetic DNA
tgtgatctgcctcagacccacagccttggcaacaggagggccttgatactcctggcacaaatgggacgaatctctcctttctcctgcctgaaggacagacatgactttggattcccccgggaggagtttgatggcaaccagttccagaaggctcaagccatctctgtcctccatgagatgatgcagcagaccttcaatctcttcagcacaaagaactcatctgctgcttgggatgagaccctcctagaaaaattttccactgaactttaccagcaactgaatgaactggaagcatgtgtgatacaggaggttggggtggaagagactcccctgatgaatgaggactccatcctggctgtgaagaaatacttccaaagaatcactctttatctgacagagaagaagtatagcccttgttcctgggaggttgtcagagcagaaatcatgagatctttctctttttcaacaaacttgcaaaaaagattaaggaggaaggaa

SEQ ID NO: 4 - DNA, 498 bp, Artificial Sequence
  Description of Artificial Sequence Synthetic DNA
tgtgatctgcctcagacccacagccttggtaacaggagggccttgatgctcctggcacaaatgggaagaatctctcctttctcctgcctgaaggacagacaagactttggattcccccaggaggagtttgatagcaaccagttccagaaggctcaagccatctctgtcctccatgagatgatgcagcagaccttcaatctcttcagcacaaaggactcatctgctgcttgggatgagaccctcctagaaaaattttccactgaactctaccagcagctgaatgacctggaagcctgcgtgatacaggaggttggggtggaagagacccccctgatgaatgtggactccatcctggctgtgaggaagtacttccaaagaatcactctttatctaatagagaggaaatacagcccttgtgcatgggaggttgtcagagcagaaatcatgagatctttctctttttcaacaaacttgcaaaaaagattaaggaggaaggaa

SEQ ID NO: 5 - DNA, 498 bp, Artificial Sequence
  Description of Artificial Sequence Synthetic DNA
tgtgatctgcctcagacccacagccttggtaacaggagggccttggtactcctggcacaaatgggaagaatctctcctttctcctgcctgaaggacagatatgatttcggattcccccaggaggagtttgatggcaaccagttccagaaggctcaagccatctctgtcctccatgagatgatccagcagaccttcaatctcttcagcacaaaggattcatctgctgcttgggatgagaccctcctagaaaaattttccactgaactttaccagcagctgaatgacctggaagcctgcgtgatacaggaggttggggtggaagagacccccctgatgaatgaggactccatcctggctgtgaagaaatacttccaaagaatcactctttatctaatagagaggaaatacagcccttgtgcatgggaggttgtcagagcagaaatcatgagatctttctctttttcaacaaacttgcaaaaaagattaaggaggaaggaa

SEQ ID NO: 6 - DNA, 498 bp, Artificial Sequence
  Description of Artificial Sequence Synthetic DNA
tgtgatctgcctcagacccacagccttggtaacaggagggccttgatgctcctggcacaaatgggaagaatctctcctttctcctgcctgaaggacagatatgatttcggattcccccaggaggagtttgatggcaaccagttccagaaggctcaagccatctctgtcctccatgagatgatccagcagaccttcaatctcttcagcacaaaggattcatctgctgcttgggatgagaccctcctagaaaaattttccactgaactttaccagcagctgaatgacttggaagcctgtgtgatacaggaggttggggtggaagagactcccctgatgaatgtggactccatcctggctgtgaggaagtacttccaaagaatcactctttatctaatagagaggaaatacagcccttgtgcatgggaggttgtcagagcagaaatcatgagatctttctctttttcaacaaacttgcaaaaaagattaaggaggaaggaa

SEQ ID NO: 7 - DNA, 498 bp, Artificial Sequence
  Description of Artificial Sequence Synthetic DNA
tgtgatctgcctcagacccacagccttggtaacaggagggccttgatactcctggcacaaatgggacgaatctctcctttctcctgcctgaaggacagacaagactttggattcccccaggaggagtttgatggcaaccggttccagaaggctcaagccatctctgtcctccatgagatgatccagcagaccttcaatctcttcagcacaaagaactcatctgctgcttgggaacagagcctcctagaaaaattttccactgaactctaccagcagctgaatgacctggaagcctgcgtgatacaggaggttggggtggaagagacccccctgatgaatgaggactccatcctggctgtgaagaaatacttccaaagaatcactctttatctaatagagaggaaatacagcccttgtgcatgggaggttgtcagagcagaaatcatgagatctttctctttttcaacaaacttgcaaaaaagattaaggaggaaggaa

SEQ ID NO: 8 - DNA, 498 bp, Artificial Sequence
  Description of Artificial Sequence Synthetic DNA
tgtgatctgcctcagacccacagccttggtaacaggagggccttgatactcctggcacaaatgggaagaatctctcctttctcctgcctgaaggacagacatgactttggattcccccaggaggagtttgatggcaaccagttccagaaggctcaagccatctctgtcctccatgagatgatccagcagaccttcaatctcttcagcacaaaggactcatctgctacttgggaacagagcctcctagaaaaattttccactgaacttaaccagcagctgaatgacctggaagcctgcgtgatacaggaggttggggtggaagagactcccctgatgaatgtggaccccatcctggctgtgaagaaatacttccaaagaatcactctctatctgacagagaagaaatacagcccttgtgcctgggaggttgtcagagcagaaatcatgagatctttctctttttcaacaaacttgcaaaaaagattaaggaggaaggaa

SEQ ID NO: 9 - DNA, 498 bp, Artificial Sequence
  Description of Artificial Sequence Synthetic DNA
tgtgatctgcctcagacccacagccttggtaacaggagggccttgatactcctggcacaaatgcgaagaatctctcctttctcctgcctgaaggacagacatgactttggattcccccaggaggagtttgatagcaaccagttccagaaggctcaagccatctctgtcctccatgagatgatccagcagaccttcaatctcttcagcacaaaggattcatctgctgcttgggaacagagcctcctagaaaaattttccactgaacttcaccagcaactgaatgaactggaagcatgtgtagtacaggaggttggggtggaagagactcccctgatgaatgaggactccatcctggctgtgaagaaatacctccaaagaatcactctttatctgacagagaagaagtatagcccttgtgcatgggaggttgtcagagcagaaatcatgagatctttctctttttcaacaaacttgcaaaaaagattaaggaggaaggaa

SEQ ID NO: 10 - DNA, 498 bp, Artificial Sequence
  Description of Artificial Sequence Synthetic DNA
tgtgatctgcctcagacccacagccttggtaacaggagggccttgatgctcctggcacaaatgggaagaatctctcctttctcctgcctgaaggacagacaagactttggattcccccaggaggagtttgatggcaaccagttccagaaggctcaagccatctctgtcctccatgagatgatccagcagaccttcaatctcttcagcacaaaggattcatctgctgcttgggaacagagcctcctagaaaaattttccactgaactctaccagcagctgaatgacctggaagcctgcgtgatacaggaggttggggtggaagagacccccctgatgaatgtggactccatcctggctgtgaggaagtacttccaaagaatcactctttatctaatagagaggaaatacagcccttgtgcatgggaggttgtcagagcagaaatcatgagatctttctctttttcaacaaacttgcaaaaaagattaaggaggaaggaa

SEQ ID NO: 11 - DNA, 498 bp, Artificial Sequence
  Description of Artificial Sequence Synthetic DNA
tgtgatctgcctcagacccacagccttggtaacaggagggccctgatactcctggcacaaatgggacgaatctctcctttctcctgcctgaaggacagatatgatttcggattcccccaggaggagtttgatggcaaccagttccagaaggctcaagccatctctgtcctccatgagatgatccagcagaccttcaatctcttcagcacaaaggattcatctgctgcttgggaacagagcctcctagaaaaattttccactgaactttaccagcagctgaatgacctggaagcctgcgtgatacaggaggttggggtggaagagacccccctaatgaatgtggactccatcctggctgtgaggaagtacttccaaagaatcactctttatctaatagagaggaaatacagcccttgtgcatgggaggttgtcagagcagaaatcatgagatctttctctttttcaacaaacttgcaaaaaagattaaggaggaaggaa

SEQ ID NO: 12 - DNA, 498 bp, Artificial Sequence
  Description of Artificial Sequence Synthetic DNA
tgtgatctgcctcagacccacagccttggtaacaggagggccttgatactcctgggacaaatgggaagaatctctcatttctcctgcctgaaggacagacatgactttggattcccccaggaggagtttgatggcaaccagttccagaaggctcaagccatctctgtcctccatgagatgatccagcagaccttcaacctcttcagcacaaaggactcatctgttgcttgggatgagaggcttctagacaaactctatactgaactttaccagcagctgaatgacctggaagcctgtgtgatgcaggaggtgtgggtgggagggactcccctgatgaatgaggactccatcctggctgtgagaaaatacttccaaagaatcactctctatctgacagagaagaaatacagcccttgtgcctgggaggttgtcagagcagaaatcatgagatctttctctttttcaacaaacttgcaaaaaagattaaggaggaaggaa

SEQ ID NO: 13 - DNA, 498 bp, Artificial Sequence
  Description of Artificial Sequence Synthetic DNA
tgtgatctgcctcagacccacagccttggtaacaggagggccttgatactcctggcacagatgggacgaatctctcctttctcctgcctgaaggacagatatgatttcggattcccccaggaggagtttgatggcaaccagttccagaaggctcaagccatctctgtcctccatgagataatgcagcagaccttcaatctcttcagcacaaagaactcatctgctgcttgggatgagaccctcctagaaaaattttccactgaactttaccagcaactgaatgaactggaagcatgtgtgatacagggggttggggtggaagagactcccctgatgaatgaggactccatcttggctgtgaggaaatacttccaaagaatcactctttatctgacagagaagaagtatagcccttgttcctgggaggttgtcagagcagaaatcatgagatctttctctttttcaacaaacttgcaaaaaagattaaggaggaaggaa

SEQ ID NO: 14 - DNA, 498 bp, Artificial Sequence
  Description of Artificial Sequence Synthetic DNA
tgtgatctgcctcagacccacagccttggtaacaggagggccttgatgctcctggcacaaatgggaagaatctcccctttctcctgcctgaaggacagatatgatttcggattcccccaggaggagtttgatggcaaccagttccagaaggctcaagccatctctgtcctccatgagatgatccagcagaccttcaatctcttcagcacaaaggattcatctgctgcttgggaacagagcctcctagaaaaattttccactggactctaccagcagctgaatgacctggaagcctgcgtgatacaggaggttggggtggaagagacccccctgatgaatgaggactccatcctggctgtgaagaaatacttccaaagaatcactctttatctgacagagaagaagtatagcccttgttcctgggaggttgtcagagcagaaatcatgagatctttctctttttcaacaaacttgcaaaaaagattaaggaggaaggaa

SEQ ID NO: 15 - DNA, 498 bp, Artificial Sequence
  Description of Artificial Sequence Synthetic DNA
tgtgatctgcctcagacccacagccttggcaacaggagggccttgatactcctggcacaaatgggaagaatctctcctttctcctgcctgaaggacagacatgactttggattaccccaggaggagtttgatggcaaccagttccagaaggctcaagccatctctgtcctccatgagatgatccagcagaccttcaatctcttcagcacaaagaactcatctgctgcttgggatgagaccctcctagaaaaattttccactgaactttaccagcaactgaataacctggaagcatgtgtgatacaggaggttgggatggaagagactcccctgatgaatgtggactccatcctggctgtgaagaaatacttccaaagaatcactctttatctgacagagaagaagtatagcccttgtgcctgggaggttgtcagagcagaaatcatgagatctttctctttttcaacaaacttgcaaaaaagattaaggaggaaggaa

SEQ ID NO: 16 - DNA, 498 bp, Artificial Sequence
  Description of Artificial Sequence Synthetic DNA
tgtgatctgcctcagacccacagccttggtaacaggagggccttgatactcctggcacaaatgggacgaatctctcctttctcctgcctgaaggacagatatgatttcggattcccccaggaggagtttgatggcaaccagttccagaaggctcaagccatctctgtcctccatgagatgatgcagcagaccttcaatctcttcagcacaaagaactcatctgctgcttgggatgagaccctcctagaaaaattttccactgaactttaccagcaactgaatgaactggaagcatgtgtgatacaggaggttggggtggaagagactcccctgatgaatgaggactccatcctggctgtgaagaaatacttccaaagaatcactctttatctgacagagaagaagtatagcccttgttcctgggaggttgtcagagcagaaatcatgagatctttctctttttcaacaaacttgcaaaaaagattaaggaggaaggaa

SEQ ID NO: 17 - DNA, 498 bp, Artificial Sequence
  Description of Artificial Sequence Synthetic DNA
tgtgatctgcctcagacccacagccttggtaacaagagggccatgatgctcctggcacaaatgggaagaacctctcctttctcctgtctgaaggacagacatgactttggattcccccaggaggagtttgatggcaaccagttccagagggctcaagccatctttgtcctccatgagatgatccagcagaccttcaatttcttcagcacaaaggactcatctgctgcttgggaacagagcctcctagaaaaattttccactgaacttaaccagcagctgaatgacctggaagcctgcgtgatacaggaagttggggtggaagagactcccctgatgaatgaggactccatcctggctgtgaagaaatacttccaaagaatcactctttatctgacagagaagaaatacagcccttgtgcctgggaggttgtcagagcagaaatcatgagatctttctctttttcaacaaacttgcaaaaaagattaaggaggaaggaa

SEQ ID NO: 18 - DNA, 498 bp, Artificial Sequence
  Description of Artificial Sequence Synthetic DNA
tgtgatctgcctcagacccacagccttggtaacagtagggccttgatgctcctggcacaaatgggaagaatctcccctttctcctgcctgaaggacagacatgatttcggattcccccaggaggagtttgatggcaaccagttccagaaggctcaagccatctctgccttccatgagatgatccagcagaccttcaatctcttcagcacaaaggattcatctgctgcttgggaacagaacctcctagaaaaattttccactgaactttaccagcaactgaataacctggaagcatgtgtgatacaggaggttgggatggaagagactcccctgatgaatgtggactccatcctggctgtgaggaagtacttccaaagaatcactctttatctaatagagaggaaatacagcccttgtgcctgggaggttgtcagagcagaaatcatgagatctttctctttttcaacaaacttgcaaaaaagattaaggaggaaggaa

SEQ ID NO: 19 - DNA, 498 bp, Artificial Sequence
  Description of Artificial Sequence Synthetic DNA
tgtgatctgcctcagacccacagccttggtaacaggagggccttgatactcctggcacaaatgggaagaatctctcatttctcctgcctgaaggacagacatgatttcggattcccccaggaggagtttgatggccaccagttccagaagactcaagccatctctgtcctccatgagatgatccagcagaccttcaatctcttcagcacaaaggactcatctgctgcttgggaacagagcctcctagaaaaattttccactgaactttaccagcaactgaatgacctggaagcatgtgtgatacaggaggttggggtggaagagactcccctgatgaatgaggactccatcctggctgtgaagaaatacttccaaagaatcactctttatctgatggagaagaaatacagcccttgtgcctgggaggttgtcagagcagaaatcatgagatctttctctttttcaacaaacttgcaaaaaagattaaggaggaaggaa

SEQ ID NO: 20 - DNA, 498 bp, Artificial Sequence
  Description of Artificial Sequence Synthetic DNA
tgtgatctgcctcagacccacagccttggtaacaggagggccatgatgctcctggcacaaatgagcagaatctctccttcctcctgtctgatggacagacatgactttgaatttccccaggaggaatttgatgataaacagttccagaaggctccagccatctctgtcctccatgaggtgattcagcagaccttcaatctcttcagcacagaggactcatctgctgcttgggaacagaccctcctagaaaaattttccactgaactttaccagcaactgaatgacctggaagcatgtgtgatgcaggaggagagggtgggagaaactcccctgatgaatgcggactccatcttggctgtgaggaaatacttccaaagaatcactctttatctgacaaagaagaagtatagcccttgttcctgggaggttgtcagagcagaaatcatgagatctttctctttttcaacaaacttgcaaaaaagattaaggaggaaggaa

SEQ ID NO: 21 - DNA, 498 bp, Artificial Sequence
  Description of Artificial Sequence Synthetic DNA
tgtgatctgcctcagacccacagccttggtaacaggagggccttgatactcctggcacaaatgggaagaatctctcatttctcctgcctgaaggacagatatgatttcggattcccccaggaggtgtttgatggcaaccagttccagaaggcccaagccatctctgccttccatgagatgatgcagcagaccttcaatctcttcagcacagaggactcatctgctgcttgggaacagagcctcctagaaaaattttccactgaacttcaccagcaactgaatgacctggaagcctgtgtgatacaggaggttggggtggaagagactcccctgatgaatgaggactccatcctggctgtgaggaaatactttcaaagaatcactctttatctaatggagaagaaatacagcccttgtgcctgggaggttgtcagagcagaaatcatgagatctttctctttttcaacaaacttgcaaaaaagattaaggaggaaggaa

SEQ ID NO: 22 - DNA, 498 bp, Artificial Sequence; identical to SEQ ID NO: 19
  Description of Artificial Sequence Synthetic DNA

SEQ ID NO: 23 - DNA, 498 bp, Artificial Sequence
  Description of Artificial Sequence Synthetic DNA
tgtgatctgcctcagacccacagccttggtaacaggaggactttgatgataatggcacaaatgggaagaatctctcctttctcctgcctgaaggacagacatgactttggatttccccaggaggagtttgatggcaaccagttccagaaggctcaagccatctctgtcctccatgagatgatccagcagaccttcaatctcttcagcacaaaggactcatctgctacttgggaacagagcctcctagaaaaattttccactgaacttaaccagcagctgaatgacctggaagcctgcgtgatacaggaggctggggtggaagagactcccctgatgaatgtggactccatcctggctgtgaagaaatacttccaaagaatcactctttatctaacagagaagaaatacagcccttgtgcctgggaggttgtcagagcagaaatcatgagatctttctctttttcaacaaacttgcaaaaaagattaaggaggaaggaa

SEQ ID NO: 24 - DNA, 498 bp, Artificial Sequence
  Description of Artificial Sequence Synthetic DNA
tgtgatctgcctcagacccacagccttggtaacaggagggccttgatactcctggcacaaatgggaagaatctctcatttctcctgcctgaaggacagatatgatttcggattcccccaggaggtgtttgatggcaaccagttccagaaggctcaagccatctctgccttccatgagatgatccagcagaccttcaatctcttcagcacaaaggactcatctgctacttgggaacagagcctcctagaaaaattttccactgaactttaccagcaactgaataacctggaagcatgtgtgatacaggaggttggggtggaagagactcccctgatgaatgaggactccatcctggctgtgaggaaatactttcaaagaatcactctttatctgatggagaagaaatacagcccttgtgcctgggaggttgtcagagcagaaatcatgagatctttctctttttcaacaaacttgcaaaaaagattaaggaggaaggaa

SEQ ID NO: 25 - DNA, 498 bp, Artificial Sequence
  Description of Artificial Sequence Synthetic DNA
tgtgatctgcctcagacccacagccttggtaacaggagggccttgatactcctggcacaaatgggaagaatctctcctttctcctgcctgaaggacagacatgactttggatttcctcaggaggagtttgatggcaaccagtcccagaaggctcaagccatctctgtcctccatgagatgatccagcagaccttcaatctcttcagcacaaaggactcatctgatacttgggatgcgacccttttagaaaaattttccactgaacttaaccagcagctgaatgacctggaagcctgcgtgatacaggaggttggggtggaagagacccccctgatgaatgtggactccatcctggctgtgaagaaatacttccaaagaatcactctttatctgacagagaagaaatacagcccttgtgcctgggaggttgtcagagcagaaatcatgagatctttctctttttcaacaaacttgcaaaaaagattaaggaggaaggaa

SEQ ID NO: 26 - DNA, 498 bp, Artificial Sequence; identical to SEQ ID NO: 7
  Description of Artificial Sequence Synthetic DNA

SEQ ID NO: 27 - DNA, 498 bp, Artificial Sequence
  Description of Artificial Sequence Synthetic DNA
tgtgatctgcctcagacccacagccttggtaacaggagggccttgatactcctggcacaaatgggaagagtctctcctttctcctgcctgaaggacagacatgactttggattcccccaggaggagtttgatggcaaccagttccagaaggctcaagccatctctgccttccatgagatgatccagcagaccttcaatctcttcagcacaaaggactcatctgctacttgggaacagagcctcctagaaaaattttccactgaactttaccagcaactgaataacctggaagcctgcgtgatacaggaggttggggtggaagagactcccctgatgaatgtggactccatcctggctgtgaagaaatacttccgaagaatcactctctatctgacagagaagaaatacagcccttgtgcctgggaggttgtcagagcagaaatcatgagatctttctctttttcaacaaacttgcaaaaaagattaaggaggaaggaa

SEQ ID NO: 28 - DNA, 498 bp, Artificial Sequence
  Description of Artificial Sequence Synthetic DNA
tgtgatctgcctcagacccacagccttggtaacaggcgggccttgatactcctggcacaaatgggaagaatctctcctttctcctgtctgaaggacagacatgacttcagatttccccaggaggagtttgatggcaaccagttccagaaggctcaagccatctctgtcctccatgagatgatccagcagaccttcaatctcttcagcacaaaggactcatctgctacttgggaacagagcctcctagaaaaattttccactgaactttaccagcaactgaataacctggaagcttgcgtgatacaggaggttggggtggaagagactcccctgatgaatgtggactctatcctggctgtgaagaaatacttccaaagaatcactctttatctgacagagaggaaatacagcccttgtgcctgggaggttgtcagagcagaaatcatgagatctttctctttttcaacaaacttgcaaaaaagattaaggaggaaggaa

SEQ ID NO: 29 - DNA, 498 bp, Artificial Sequence
  Description of Artificial Sequence Synthetic DNA
tgtgatctgcctcagacccacagccttggtaacaggagggccttgatactcctggcacaaatgggaagaatctctcctttctcctgcctgaaggacagacatgactttggattcccccaggaggagtttgatggcaaccagttccagaaggctcaagccatctctgtcctccatgagatgatccagcagactttcaatctcttcagcacaaaggactcatctgctacttgggaacagagcctcctagaaaaattttccactgaacttaaccagcagctgaatgacctggaagcctgcgtgatacaggaggttggggtggaagagactcccctggtgaatgtggactccatcctggctgtgaagaaatacttccaaagaatcactctttatctgacagagaagaaatacagcccttgtgcctgggaggttgtcagagcagaaatcatgagatctttctctttttcaacaaacttgcaaaaaagattaaggaggaaggaa

SEQ ID NO: 30 - DNA, 498 bp, Artificial Sequence
  Description of Artificial Sequence Synthetic DNA
tgtgatctgcctcagacccacagccttggtaacaggaggcccttgatactcctggcacaaatgggaagaatctctcctttctcctgcctgaaggacagacaggacttcggattcccccaggaggagtttgatggcaaccagttccagaaggctcaagccatctctgtcctccatgagatgatgcagcagaccttcaatctcttcagcacaaagaactcatctgctgcttgggaacagagcctcctagaaaaattttccactgaactccaccagcaactgaatgaactggaagcatgtgtgatacaggaggttggggtggaagagactcccctgatgaatgtggactccatcctggctgtgaagaaatacttccaaagaatcactctttatctaatagagaggaaatacagcccttgtgcatgggaggttgtcagagcagaaatcatgagatctttctctttttcaacaaacttgcaaaaaagattaaggaggaaggaa

SEQ ID NO: 31 - DNA, 498 bp, Artificial Sequence
  Description of Artificial Sequence Synthetic DNA
tgtgatctgcctcagacccacagccctggtaacaggagggccttgatgctcctggcacaaatgggacgaatctctcctttctcctgcctgaaggacagatatgatttcggattcccccagggggagtttgatggcaaccagttccagaaggctcaagccatctctgtcctccatgagatgatgcagcagaccttcaatctcttcagcacaaaggattcatctgctgcttgggaacagagcctcctagaaaaattttccactgaactctaccggcagctgaatgacctggaagcctgtgtgatacaggaggttggggtggaagagacccccctgatgaatgtggactccatcctggctgtgaggaagtacttccaaagaatcactctttatctgacagagaagaagcatagcccttgttcctgggaggttgtcagagcagaaatcatgagatctttctctttttcaacaaacttgcaaaaaagattaaggaggaaggaa

SEQ ID NO: 32 - DNA, 498 bp, Artificial Sequence
  Description of Artificial Sequence Synthetic DNA
tgtgatctgcctcagacccacagccttggtaacaggagggccttgatactcctggcacaaatgggaagaatctctcctttctcctgcctgaaggacagacatgactttggacttccccaggaggagtttgatggcaaccagttccagaagactcaagccatctctgtcctccatgagatgatccagcagaccttcaatctcttcagcacaaaggactcatctgatacttgggaacagagcctcctagaaaaattctacattgaacttttccagcagctgaatgacctggaagcctgcgtgatacaggaggttggggtggaagagactcccctgatgaatgtggactccatcctggctgtgagaaaatacttccaaagaatcactctttatctgacagaggagaaatacagcccttgtgcctgggaggttgtcagagcagaaatcatgagatctttctctttttcaacaaacttgcaaaaaagattaaggaggaaggaa

SEQ ID NO: 33 - DNA, 498 bp, Artificial Sequence
  Description of Artificial Sequence Synthetic DNA
tgtgatctgcctcagacccacagccttggtaacaggaggactttgatgctcatggcacaaatgaggagaatctctcctttcccccgcctgaaggacagatatgatttcggattcccccaggaggtgtttgatggcaaccagttccagaaggctcaagctatcttccttttccatgagatgatgcagcagaccttcaatctcttcagcacaaagaactcatctgctgcttgggatgagaccctcctagacaaattctacactgaactctaccagcagctgaatgacttggaagcctgtgtgatgcaggaggggagggtgggagaaactcccctgatgaatgcggactccatcttggctgtgaagaaatacttccgaagaatcactctctatctgacagagaagaaatacagcccttgtgcctgggaggctgtcagagcagaaatcatgagatctttctctttttcaacaaacttgcaaaaaagattaaggaggaaggaa

SEQ ID NO: 34 - DNA, 498 bp, Artificial Sequence
  Description of Artificial Sequence Synthetic DNA
tgtgatctgcctcagacccacagccttggtaacaggagggccttgatactcctggcacaaatgggaagaatctctcctttctcctgcctgaaggacagacatgactttggattcccccaggaggagtttgatggcaaccagttccagaaggctcaagccatctctgtcctccatgagatgatccagcagaccttcaatctcttcagcacaaaggactcatctgctacttgggaacagagcctcctagaaaaattttccactgaacttaaccagcagctgaatgacctagaagcctgtgtgacacaggaggttggggtggaagagactcccctgatgaatgaggactctatcctggctgtgaagaaatacttccaaagaatcactctttatctgacagagaagaaatacagcccttgtgcctgggaggttgtcagagcagaaatcatgagatctttctctttttcaacaaacttgcaaaaaagattaaggaggaaggaa

SEQ ID NO: 35 - DNA, 498 bp, Artificial Sequence
  Description of Artificial Sequence Synthetic DNA
tgtgatctgcctcagacccacagccttggtaacaggagggccttgatactcctggcacaaatgggaagaatctctcctttctcctgcctgaaggacagatatgatttcggattcccccaggaggagtttgatggcaaccagctccagaaggctcaagccatctctgtcctccatgagatgatccagcagaccttcaatctcttcagcacaaaggattcatctgctgcttgggaacagagcctcctagaaaaattttccactgaacttaaccagcagctgaatgacctggaagcctgcgtgatacaggaggttggagtggaagagactcccctgatgaatgtggactccatcctggctgtgaagaaatacttccaaagaatcactctttatctgacagagaggaaatacagcccttgtgcctgggaggttgtcagagcagaaatcatgagatctttctctttttcaacaaacttgcaaaaaagattaaggaggaaggaa

SEQ ID NO: 36 - protein, 166 aa, Artificial Sequence
  Description of Artificial Sequence Synthetic amino acid
CDLPQTHSLGNRRALMLLAQMGRISPFSCLKDRQDFGFPQEEFDGNQFQKAQAISVLHEMIQQTFNLFSTKDSSAAWEQTLLEKFSTELYQQLNDLEACVIQEVGVKETPLMNVDSILAVRKYFQRITLYLIERKYSPCAWEVVRAEIMRSFSFSTNLQKRLRRKE

SEQ ID NO: 37 - protein, 166 aa, Artificial Sequence
  Description of Artificial Sequence Synthetic amino acid
CDLPQTHSLGDRRAMILLAQMGRISPFSCLKDRYDFGFPQEEFDGNQFQKAQAISVLHEMIQQTFNLFSTKDSSAAWEQSLLEKFSTELYQQLNELEACVIQEVGVGETPLMNGDSILAVKKYFQRITLYLIERKYSPCAWEVVRAEIMRSFSFSTNLQKRLRRKE

SEQ ID NO: 38 - protein, 166 aa, Artificial Sequence
  Description of Artificial Sequence Synthetic amino acid
CDLPQTHSLGNRRALILLAQMGRISPFSCLKDRHDFGFPREEFDGNQFQKAQAISVLHEMMQQTFNLFSTKNSSAAWDETLLEKFSTELYQQLNELEACVIQEVGVEETPLMNEDSILAVKKYFQRITLYLTEKKYSPCSWEVVRAEIMRSFSFSTNLQKRLRRKE

SEQ ID NO: 39 - protein, 166 aa, Artificial Sequence
  Description of Artificial Sequence Synthetic amino acid
CDLPQTHSLGNRRALMLLAQMGRISPFSCLKDRQDFGFPQEEFDSNQFQKAQAISVLHEMMQQTFNLFSTKDSSAAWDETLLEKFSTELYQQLNDLEACVIQEVGVEETPLMNVDSILAVRKYFQRITLYLIERKYSPCAWEVVRAEIMRSFSFSTNLQKRLRRKE

SEQ ID NO: 40 - protein, 166 aa, Artificial Sequence
  Description of Artificial Sequence Synthetic amino acid
CDLPQTHSLGNRRALVLLAQMGRISPFSCLKDRYDFGFPQEEFDGNQFQKAQAISVLHEMIQQTFNLFSTKDSSAAWDETLLEKFSTELYQQLNDLEACVIQEVGVEETPLMNEDSILAVKKYFQRITLYLIERKYSPCAWEVVRAEIMRSFSFSTNLQKRLRRKE

SEQ ID NO: 41 - protein, 166 aa, Artificial Sequence
  Description of Artificial Sequence Synthetic amino acid
CDLPQTHSLGNRRALMLLAQMGRISPFSCLKDRYDFGFPQEEFDGNQFQKAQAISVLHEMIQQTFNLFSTKDSSAAWDETLLEKFSTELYQQLNDLEACVIQEVGVEETPLMNVDSILAVRKYFQRITLYLIERKYSPCAWEVVRAEIMRSFSFSTNLQKRLRRKE

SEQ ID NO: 42 - protein, 166 aa, Artificial Sequence
  Description of Artificial Sequence Synthetic amino acid
CDLPQTHSLGNRRALILLAQMGRISPFSCLKDRQDFGFPQEEFDGNRFQKAQAISVLHEMIQQTFNLFSTKNSSAAWEQSLLEKFSTELYQQLNDLEACVIQEVGVEETPLMNEDSILAVKKYFQRITLYLIERKYSPCAWEVVRAEIMRSFSFSTNLQKRLRRKE

SEQ ID NO: 43 - protein, 166 aa, Artificial Sequence
  Description of Artificial Sequence Synthetic amino acid
CDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVIQEVGVEETPLMNVDPILAVKKYFQRITLYLTEKKYSPCAWEVVRAEIMRSFSFSTNLQKRLRRKE

SEQ ID NO: 44 - protein, 166 aa, Artificial Sequence
  Description of Artificial Sequence Synthetic amino acid
CDLPQTHSLGNRRALILLAQMRRISPFSCLKDRHDFGFPQEEFDSNQFQKAQAISVLHEMIQQTFNLFSTKDSSAAWEQSLLEKFSTELHQQLNELEACVVQEVGVEETPLMNEDSILAVKKYLQRITLYLTEKKYSPCAWEVVRAEIMRSFSFSTNLQKRLRRKE

SEQ ID NO: 45 - protein, 166 aa, Artificial Sequence
  Description of Artificial Sequence Synthetic amino acid
CDLPQTHSLGNRRALMLLAQMGRISPFSCLKDRQDFGFPQEEFDGNQFQKAQAISVLHEMIQQTFNLFSTKDSSAAWEQSLLEKFSTELYQQLNDLEACVIQEVGVEETPLMNVDSILAVRKYFQRITLYLIERKYSPCAWEVVRAEIMRSFSFSTNLQKRLRRKE

SEQ ID NO: 46 - protein, 166 aa, Artificial Sequence
  Description of Artificial Sequence Synthetic amino acid
CDLPQTHSLGNRRALILLAQMGRISPFSCLKDRYDFGFPQEEFDGNQFQKAQAISVLHEMIQQTFNLFSTKDSSAAWEQSLLEKFSTELYQQLNDLEACVIQEVGVEETPLMNVDSILAVRKYFQRITLYLIERKYSPCAWEVVRAEIMRSFSFSTNLQKRLRRKE

SEQ ID NO: 47 - protein, 166 aa, Artificial Sequence
  Description of Artificial Sequence Synthetic amino acid
CDLPQTHSLGNRRALILLGQMGRISHFSCLKDRHDFGFPQEEFDGNQFQKAQAISVLHEMIQQTFNLFSTKDSSVAWDERLLDKLYTELYQQLNDLEACVMQEVWVGGTPLMNEDSILAVRKYFQRITLYLTEKKYSPCAWEVVRAEIMRSFSFSTNLQKRLRRKE

SEQ ID NO: 48 - protein, 166 aa, Artificial Sequence
  Description of Artificial Sequence Synthetic amino acid
CDLPQTHSLGNRRALILLAQMGRISPFSCLKDRYDFGFPQEEFDGNQFQKAQAISVLHEIMQQTFNLFSTKNSSAAWDETLLEKFSTELYQQLNELEACVIQGVGVEETPLMNEDSILAVRKYFQRITLYLTEKKYSPCSWEVVRAEIMRSFSFSTNLQKRLRRKE

SEQ ID NO: 49 - protein, 166 aa, Artificial Sequence
  Description of Artificial Sequence Synthetic amino acid
CDLPQTHSLGNRRALMLLAQMGRISPFSCLKDRYDFGFPQEEFDGNQFQKAQAISVLHEMIQQTFNLFSTKDSSAAWEQSLLEKFSTGLYQQLNDLEACVIQEVGVEETPLMNEDSILAVKKYFQRITLYLTEKKYSPCSWEVVRAEIMRSFSFSTNLQKRLRRKE

SEQ ID NO: 50 - protein, 166 aa, Artificial Sequence
  Description of Artificial Sequence Synthetic amino acid
CDLPQTHSLGNRRALILLAQMGRISPFSCLKDRHDFGLPQEEFDGNQFQKAQAISVLHEMIQQTFNLFSTKNSSAAWDETLLEKFSTELYQQLNNLEACVIQEVGMEETPLMNVDSILAVKKYFQRITLYLTEKKYSPCAWEVVRAEIMRSFSFSTNLQKRLRRKE

SEQ ID NO: 51 - protein, 166 aa, Artificial Sequence
  Description of Artificial Sequence Synthetic amino acid
CDLPQTHSLGNRRALILLAQMGRISPFSCLKDRYDFGFPQEEFDGNQFQKAQAISVLHEMMQQTFNLFSTKNSSAAWDETLLEKFSTELYQQLNELEACVIQEVGVEETPLMNEDSILAVKKYFQRITLYLTEKKYSPCSWEVVRAEIMRSFSFSTNLQKRLRRKE

SEQ ID NO: 52 - protein, 166 aa, Artificial Sequence
  Description of Artificial Sequence Synthetic amino acid
CDLPQTHSLGNKRAMMLLAQMGRTSPFSCLKDRHDFGFPQEEFDGNQFQRAQAIFVLHEMIQQTFNFFSTKDSSAAWEQSLLEKFSTELNQQLNDLEACVIQEVGVEETPLMNEDSILAVKKYFQRITLYLTEKKYSPCAWEVVRAEIMRSFSFSTNLQKRLRRKE

SEQ ID NO: 53 - protein, 166 aa, Artificial Sequence
  Description of Artificial Sequence Synthetic amino acid
CDLPQTHSLGNSRALMLLAQMGRISPFSCLKDRHDFGFPQEEFDGNQFQKAQAISAFHEMIQQTFNLFSTKDSSAAWEQNLLEKFSTELYQQLNNLEACVIQEVGMEETPLMNVDSILAVRKYFQRITLYLIERKYSPCAWEVVRAEIMRSFSFSTNLQKRLRRKE

SEQ ID NO: 54 - protein, 166 aa, Artificial Sequence
  Description of Artificial Sequence Synthetic amino acid
CDLPQTHSLGNRRALILLAQMGRISHFSCLKDRHDFGFPQEEFDGHQFQKTQAISVLHEMIQQTFNLFSTKDSSAAWEQSLLEKFSTELYQQLNDLEACVIQEVGVEETPLMNEDSILAVKKYFQRITLYLMEKKYSPCAWEVVRAEIMRSFSFSTNLQKRLRRKE

SEQ ID NO: 55 - protein, 166 aa, Artificial Sequence
  Description of Artificial Sequence Synthetic amino acid
CDLPQTHSLGNRRAMMLLAQMSRISPSSCLMDRHDFEFPQEEFDDKQFQKAPAISVLHEVIQQTFNLFSTEDSSAAWEQTLLEKFSTELYQQLNDLEACVMQEERVGETPLMNADSILAVRKYFQRITLYLTKKKYSPCSWEVVRAEIMRSFSFSTNLQKRLRRKE

SEQ ID NO: 56 - protein, 166 aa, Artificial Sequence
  Description of Artificial Sequence Synthetic amino acid
CDLPQTHSLGNRRALILLAQMGRISHFSCLKDRYDFGFPQEVFDGNQFQKAQAISAFHEMMQQTFNLFSTEDSSAAWEQSLLEKFSTELHQQLNDLEACVIQEVGVEETPLMNEDSILAVRKYFQRITLYLMEKKYSPCAWEVVRAEIMRSFSFSTNLQKRLRRKE

SEQ ID NO: 57 - protein, 166 aa, Artificial Sequence
  Description of Artificial Sequence Synthetic amino acid
CDLPQTHSLGNRRALILLAQMGRISPFSCLKDRHDFRFPQEEFDGNQLQKTQAISVLHEMIQQTFNLFSTKDSSATWEQSLLEKFSTELNQQLNDLEACVIQGVGVEETPPMNVDSILAVKKYFQRITLYLTEKKYSPCAWEVVRAEIMRSFSFSTNLQKRLRRKE

SEQ ID NO: 58 - protein, 166 aa, Artificial Sequence
  Description of Artificial Sequence Synthetic amino acid
CDLPQTHSLGNRRTLMIMAQMGRISPFSCLKDRHDFGFPQEEFDGNQFQKAQAISVLHEMIQQTFNLFSTKDSSATWEQSLLEKFSTELNQQLNDLEACVIQEAGVEETPLMNVDSILAVKKYFQRITLYLTEKKYSPCAWEVVRAEIMRSFSFSTNLQKRLRRKE

SEQ ID NO: 59 - protein, 166 aa, Artificial Sequence
  Description of Artificial Sequence Synthetic amino acid
CDLPQTHSLGNRRALILLAQMGRISHFSCLKDRYDFGFPQEVFDGNQFQKAQAISAFHEMIQQTFNLFSTKDSSATWEQSLLEKFSTELYQQLNNLEACVIQEVGVEETPLMNEDSILAVRKYFQRITLYLMEKKYSPCAWEVVRAEIMRSFSFSTNLQKRLRRKE

SEQ ID NO: 60 - protein, 166 aa, Artificial Sequence
  Description of Artificial Sequence Synthetic amino acid
CDLPQTHSLGNRRALILLAQMGRISPFSCLKDRHDFGFPQEEFDGNQSQKAQAISVLHEMIQQTFNLFSTKDSSDTWDATLLEKFSTELNQQLNDLEACVIQEVGVEETPLMNVDSILAVKKYFQRITLYLTEKKYSPCAWEVVRAEIMRSFSFSTNLQKRLRRKE

SEQ ID NO: 61 - protein, 166 aa, Artificial Sequence
  Description of Artificial Sequence Synthetic amino acid
CDLPQTHSLGNRRALILLAQMRRISPFSCLKDRHDFGFPQEEFDGNQFQKAQAISAFHEMIQQTFNLFSTKDSSAAWEQSLLEKFSTELYQQLNNLEACVIQEVGMEETPLMNEDSILAVKKYFQRITLYLTEKKYSPCAWEVVRAEIMRSFSFSTNLQKRLRRKE

SEQ ID NO: 62 - protein, 166 aa, Artificial Sequence
  Description of Artificial Sequence Synthetic amino acid
CDLPQTHSLGNRRALILLAQMGRVSPFSCLKDRHDFGFPQEEFDGNQFQKAQAISAFHEMIQQTFNLFSTKDSSATWEQSLLEKFSTELYQQLNNLEACVIQEVGVEETPLMNVDSILAVKKYFRRITLYLTEKKYSPCAWEVVRAEIMRSFSFSTNLQKRLRRKE

SEQ ID NO: 63 - protein, 166 aa, Artificial Sequence
  Description of Artificial Sequence Synthetic amino acid
CDLPQTHSLGNRRALILLAQMGRISPFSCLKDRHDFRFPQEEFDGNQFQKAQAISVLHEMIQQTFNLFSTKDSSATWEQSLLEKFSTELYQQLNNLEACVIQEVGVEETPLMNVDSILAVKKYFQRITLYLTERKYSPCAWEVVRAEIMRSFSFSTNLQKRLRRKE

SEQ ID NO: 64 - protein, 166 aa, Artificial Sequence
  Description of Artificial Sequence Synthetic amino acid
CDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVIQEVGVEETPLVNVDSILAVKKYFQRITLYLTEKKYSPCAWEVVRAEIMRSFSFSTNLQKRLRRKE

SEQ ID NO: 65 - protein, 166 aa, Artificial Sequence
  Description of Artificial Sequence Synthetic amino acid
CDLPQTHSLGNRRPLILLAQMGRISPFSCLKDRQDFGFPQEEFDGNQFQKAQAISVLHEMMQQTFNLFSTKNSSAAWEQSLLEKFSTELHQQLNELEACVIQEVGVEETPLMNVDSILAVKKYFQRITLYLIERKYSPCAWEVVRAEIMRSFSFSTNLQKRLRRKE

SEQ ID NO: 66 - protein, 166 aa, Artificial Sequence
  Description of Artificial Sequence Synthetic amino acid
CDLPQTHSPGNRRALMLLAQMGRISPFSCLKDRYDFGFPQGEFDGNQFQKAQAISVLHEMMQQTFNLFSTKDSSAAWEQSLLEKFSTELYRQLNDLEACVIQEVGVEETPLMNVDSILAVRKYFQRITLYLTEKKHSPCSWEVVRAEIMRSFSFSTNLQKRLRRKE

SEQ ID NO: 67 - protein, 166 aa, Artificial Sequence
  Description of Artificial Sequence Synthetic amino acid
CDLPQTHSLGNRRALILLAQMGRISPFSCLKDRHDFGLPQEEFDGNQFQKTQAISVLHEMIQQTFNLFSTKDSSDTWEQSLLEKFYIELFQQLNDLEACVIQEVGVEETPLMNVDSILAVRKYFQRITLYLTEEKYSPCAWEVVRAEIMRSFSFSTNLQKRLRRKE

SEQ ID NO: 68 - protein, 166 aa, Artificial Sequence
  Description of Artificial Sequence Synthetic amino acid
CDLPQTHSLGNRRTLMLMAQMRRISPFPRLKDRYDFGFPQEVFDGNQFQKAQAIFLFHEMMQQTFNLFSTKNSSAAWDETLLDKFYTELYQQLNDLEACVMQEGRVGETPLMNADSILAVKKYFRRITLYLTEKKYSPCAWEAVRAEIMRSFSFSTNLQKRLRRKE

SEQ ID NO: 69 - protein, 166 aa, Artificial Sequence
  Description of Artificial Sequence Synthetic amino acid
CDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVTQEVGVEETPLMNEDSILAVKKYFQRITLYLTEKKYSPCAWEVVRAEIMRSFSFSTNLQKRLRRKE

SEQ ID NO: 70 - protein, 166 aa, Artificial Sequence
  Description of Artificial Sequence Synthetic amino acid
CDLPQTHSLGNRRALILLAQMGRISPFSCLKDRYDFGFPQEEFDGNQLQKAQAISVLHEMIQQTFNLFSTKDSSAAWEQSLLEKFSTELNQQLNDLEACVIQEVGVEETPLMNVDSILAVKKYFQRITLYLTERKYSPCAWEVVRAEIMRSFSFSTNLQKRLRRKE

SEQ ID NO: 71 - protein, 166 aa, Artificial Sequence
  Description of Artificial Sequence Synthetic amino acid
CDLPQTHSLGXXRAXXLLXQMXRXSXFSCLKDRXDFGXPXEEFDXXXFQXXQAIXXXHEXXQQTFNXFSTKXSSXXWXXXLLXKXXTXLXQQLNXLEACVXQXVXXXXTPLMNXDXILAVXKYXQRITLYLXEXKYSPCXWEVVRAEIMRSFSFSTNLQKRLRRKE

SEQ ID NO: 72 - DNA, 498 bp, Artificial Sequence
  Description of Artificial Sequence Synthetic DNA
tgtgatctgcctcagacccacagccttggtaacaggagggccttgatactcctggcacaaatgggaagaatctctcctttctcctgtctgatggacagacatgactttggatttccccaggaggagtttgatgacaaccagttccagaaggctcaagccatctctgtcctccatgagatgatccaacagaccttcaatctcttcagcacaaaggactcatctgctacttgggatgagacacttctagacaaattctacactgaactttaccagcagctgaatgacctggaagcctgcgtgatacaggaggttggggtggaagagactcccctgatgaatgaggactccatcttggctgtgaagaaatacttccgaagaatcactctctatctgacagagaagaaatacagcccttgtgcctgggaggttgtcagagcagaaatcatgagatctttctctttttcaacaaacttgcaaaaaagattaaggaggaaggaa

SEQ ID NO: 73 - DNA, 498 bp, Artificial Sequence
  Description of Artificial Sequence Synthetic DNA
tgtgatctgcctcagacccacagccttggtaacaggagggccttgatactcctggcacaaatgggaagaatctctcctttctcctgcctgaaggacagacatgactttggattcccccaggaggagtttgatggcaaccagttccagaaggctcaaggcatctctgtcctccatgagatgatccagcagaccttccatctcttcagcacaaaggactcatctgctacttgggaacagagcctcctagaaaaattttccactgaacttaaccagcagctgaatgacctggaagcctgcgtgatacaggaggttggggtggaagagactcccctgatgaatgtggactccatcctggctgtgaagaaatacttccgaagaatcactctttatctgacagagaagaaatacagcccttgtgcctgggaggttgtcagagcagaaatcatgagatctttctctttttcaacaaacttgcaaaaaagattaaggaggaaggaa

SEQ ID NO: 74 - DNA, 498 bp, Artificial Sequence
  Description of Artificial Sequence Synthetic DNA
tgtgatctgcctcagacccacagccttggtaacaggaggactttgatgataatggcacaaatgggaagaatctctcctttctcctgcctgaaggacagacatgactttggatttcctcaggaggagtttgatggcaaccagttccagaaggctcaagccatctctgtcctccatgagatgatccagcagaccttcaatctcttcagcacaaaggactcatctgctacttgggatgagacacttctagacaaattctacactgaactttaccagcagctgaatgacctggaagcctgtatgatgcaggaggttggagtggaagacactcctctgatgaatgtggactctatcctgactgtgagaaaatactttcgaagaatcactctttatctgacagagaagaaatacagcccttgtgcctgggaggttgtcagagcagaaatcatgagatctttctctttttcaacaaacttgcaaaaaagattaaggaggaaggaa

SEQ ID NO: 75 - DNA, 498 bp, Artificial Sequence
  Description of Artificial Sequence Synthetic DNA
tgtgatctgcctcagacccacagcctgggtaataggagggccttgatactcctggcacaaatgggaagaatctctcctttctcctgcctgaaggacagacatgactttggattcccccaggaggagtttggtggcaaccagttccagaaggctcaagccatctctgtcctccatgagatgatccagcagaccttcaatctcttcagcacagaggactcatctgctgcttgggatgagaccctcctagacaaattctacattgaacttttccagcaactgaatgacctggaagcctgtgtgatgcaggaggagagggtgggagaaactcccctgatgaatgcggactccatcttggctgtgaagaaatacttccaaagaatcactctttatctgacagagaagaaatacagcccttgtgcctgggaggttgtcagagcagaaatcatgagatctttctctttttcaacaaacttgcaaaaaagattaaggaggaaggaa

SEQ ID NO: 76 - DNA, 498 bp, Artificial Sequence
  Description of Artificial Sequence Synthetic DNA
tgtgatctgcctcagacccacagccttggtaacaggaggactttgatgataatggcacaaatgggaagaatctctcctttctcctgcctgaaggacagacatgactttggatttcctcaggaggagtttgatggcaaccagttccagaaggctcaagccatctctgtcctccatgagatgatccagcagaccttcaatctcttcagcacaaaggactcatctgctacttgggatgagacacttctagacaaattctacactgaactttaccagcagctgaatgacctggaagcctgtatgatacaggaggttggggtggaagagactcccctgatgaatgaggactccatcttggctgtgaagaaatacttccgaagaatcactctctatctgacagagaagaaatacagcccttgtgcctgggaggttgtcagagcagaaatcatgagatctttctctttttcaacaaacttgcaaaaaagattaaggaggaaggaa

SEQ ID NO: 77 - DNA, 498 bp, Artificial Sequence
  Description of Artificial Sequence Synthetic DNA
tgtgatctgcctcagacccacagccttggtaacaggagggccttgatactcctggcacaaatgggaagaatctctcctttctcctgtctgatggacagacatgactttggatttccccaggaggagtttgatgacaaccagttccagaaggctcaagccatctctgtcctccatgagatgatccaacagaccttcaatctcttcagcacaaaggactcatctgctacttgggatgagacacttctagacaaattctacactgaactttaccagcagctgaatgacctggaagcctgtatgatgcaggaggttggagtggaagacactcctctgatgaatgtggactctatcctgactgtgaagaaatacttccgaagaatcactctttatctgacagagaagaaatacagcccttgtgcctgggaggttgtcagagcagaaatcatgagatctttctctttttcaacaaacttgcaaaaaagattaaggaggaaggaa

SEQ ID NO: 78 - DNA, 498 bp, Artificial Sequence
  Description of Artificial Sequence Synthetic DNA
tgtgatctgcctcagacccacagccttggtaacaggaggactttgatgataatggcacaaatgggaagaatctctcctttctcctgcctgaaggacagacatgactttggatttcctcaggaggagtttgatggcaaccagttccagaaggctcaagccatctctgtcctccatgagatgatccagcagaccttcaatctcttcagcacaaaggactcatctgctacttgggatgagacacttctagacaaattctacactgaactttaccagcagctgaatgacctggaagcctgtatgatgcaggaggttggagtggaagacactcctctgatgaatgaggactccatcttggctgtgaagaaatacttccgaagaatcactctctatctgacagagaagaaatacagcccttgtgcctgggaggttgtcagagcagaaatcatgagatctttctctttctcaacaaacttgcaaaaaagattaaggaggaaggaa

SEQ ID NO: 79 - protein, 166 aa, Artificial Sequence
  Description of Artificial Sequence Synthetic amino acid
CDLPQTHSLGNRRALILLAQMGRISPFSCLMDRHDFGFPQEEFDDNQFQKAQAISVLHEMIQQTFNLFSTKDSSATWDETLLDKFYTELYQQLNDLEACVIQEVGVEETPLMNEDSILAVKKYFRRITLYLTEKKYSPCAWEVVRAEIMRSFSFSTNLQKRLRRKE

SEQ ID NO: 80 - protein, 166 aa, Artificial Sequence
  Description of Artificial Sequence Synthetic amino acid
CDLPQTHSLGNRRALILLAQMGRISPFSCLKDRHDFGFPQEEFDGNQFQKAQGISVLHEMIQQTFHLFSTKDSSATWEQSLLEKFSTELNQQLNDLEACVIQEVGVEETPLMNVDSILAVKKYFRRITLYLTEKKYSPCAWEVVRAEIMRSFSFSTNLQKRLRRKE

SEQ ID NO: 81 - protein, 166 aa, Artificial Sequence
  Description of Artificial Sequence Synthetic amino acid
CDLPQTHSLGNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFRRITLYLTEKKYSPCAWEVVRAEIMRSFSFSTNLQKRLRRKE

SEQ ID NO: 82 - protein, 166 aa, Artificial Sequence
  Description of Artificial Sequence Synthetic amino acid
CDLPQTHSLGNRRALILLAQMGRISPFSCLKDRHDFGFPQEEFGGNQFQKAQAISVLHEMIQQTFNLFSTEDSSAAWDETLLDKFYIELFQQLNDLEACVMQEERVGETPLMNADSILAVKKYFQRITLYLTEKKYSPCAWEVVRAEIMRSFSFSTNLQKRLRRKE

SEQ ID NO: 83 - protein, 166 aa, Artificial Sequence
  Description of Artificial Sequence Synthetic amino acid
CDLPQTHSLGNRRTLMIMAQMGRISPFSCLKDRHDFGFPQEEFDGNQFQKAQAISVLHEMIQQTFNLFSTKDSSATWDETLLDKFYTELYQQLNDLEACMIQEVGVEETPLMNEDSILAVKKYFRRITLYLTEKKYSPCAWEVVRAEIMRSFSFSTNLQKRLRRKE

SEQ ID NO: 84 - protein, 166 aa, Artificial Sequence
  Description of Artificial Sequence Synthetic amino acid
CDLPQTHSLGNRRALILLAQMGRISPFSCLMDRHDFGFPQEEFDDNQFQKAQAISVLHEMIQQTFNLFSTKDSSATWDETLLDKFYTELYQQLNDLEACMMQEVGVEETPLMNVDSILTVKKYFRRITLYLTEKKYSPCAWEVVRAEIMRSFSFSTNLQKRLRRKE

SEQ ID NO: 85 - protein, 166 aa, Artificial Sequence
  Description of Artificial Sequence Synthetic amino acid
CDLPQTHSLGNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEETPLMNEDSILAVKKYFRRITLYLTEKKYSPCAWEVVRAEIMRSFSFSTNLQKRLRRKE

SEQ ID NO: 86 - DNA, 15 bp, Artificial Sequence
  Description of Artificial Sequence Synthetic DNA
tgcgacttaccacaa

SEQ ID NO: 87 - protein, 26 aa, Artificial Sequence
  Description of Artificial Sequence Synthetic amino acid
WEVVRSEIMRSFSYSTNLQRRLRRKD

SEQ ID NO: 88 - protein, 26 aa, Artificial Sequence
  Description of Artificial Sequence Synthetic amino acid
WELVRAEIVRSFSFSTNLNKRLRKKE